(19) United States
(12) Patent Application Publication
(10) Pub. No.: US 2018/0360761 A1
(43) Pub. Date: Dec. 20, 2018

(54) PHARMACEUTICAL COMPOSITION COMPRISING A POTENT INHIBITOR OF URAT1

(71) Applicants: Ardea Biosciences, Inc., San Diego, CA (US); AstraZeneca AB, Södertälje (SE)

(72) Inventors: Joanne REILAND WAKEMAN, San Diego, CA (US); Colin ROWLINGS, San Diego, CA (US); Sha LIU, San Diego, CA (US); Gerry BURKE, San Diego, CA (US); Christian VON CORSWANT, Mölndal (SE); Christer TANNERGREN, Mölndal (SE); Johan HJÄRTSTAM, Mölndal (SE)

(73) Assignees: Ardea Biosciences, Inc., San Diego, CA (US); AstraZeneca AB, Södertälje (SE)

(21) Appl. No.: 15/781,693

(22) PCT Filed: Dec. 7, 2016
(86) PCT No.: PCT/EP2016/080111

§ 371 Date: Jun. 5, 2018

Related U.S. Application Data
(60) Provisional application No. 62/264,792, filed on Dec. 8, 2015.

(51) Int. Cl. A61K 9/20; A61K 9/00; A61K 45/06; A61K 31/426; A61K 31/4418; A61K 9/50; A61K 9/48

(52) U.S. Cl. CPC A61K 9/2081; A61K 9/0053; A61K 45/06; A61K 31/426; A61K 9/5047; A61K 9/5042; A61K 9/485; A61K 9/4858; A61K 31/4418

(57) ABSTRACT

The present invention relates to pharmaceutical compositions containing 2-((3-(4-cyanonaphthalen-1-yl)pyridine-4-yl)thio)-2-methylpropanoic acid or a pharmaceutically acceptable salt (hereinafter referred to as the “Agent”), more particularly to orally deliverable compositions containing the Agent; to the use of said compositions as a medicament; and to processes for the preparation of said compositions.

The present invention relates to pharmaceutical compositions containing 2-((3-(4-cyanonaphthalen-1-yl)pyridine-4-yl)thio)-2-methylpropanoic acid or a pharmaceutically acceptable salt thereof (hereinafter referred to as the “Agent”), more particularly to orally deliverable compositions containing the Agent; to the use of said compositions as a medicament; and to processes for the preparation of said compositions.

The Agent is disclosed in International Patent Publication WO 2011/159839 and is a potent inhibitor of URAT1. The Agent is a compound with the structure of the Formula I:

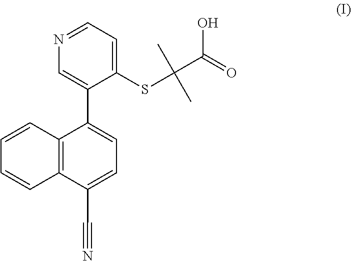 [SMILES: CC(C)(Sc1ccncc1-c1ccc(C#N)c2ccccc12)C(=O)O]

The Agent is a selective uric acid reabsorption inhibitor and is expected to be useful in the treatment of diseases or medical conditions mediated alone or in part by uric acid metabolism. Disorders of uric acid metabolism include, but are not limited to, polycythemia, myeloid metaplasia, gout, a recurrent gout attack, gouty arthritis, hyperuricaemia, hypertension, a cardiovascular disease, coronary heart disease, Lesch-Nyhan syndrome, Kelley-Seegmiller syndrome, kidney disease, kidney stones, kidney failure, joint inflammation, arthritis, urolithiasis, plumbism, hyperparathyroidism, psoriasis and sarcoidosis. The Agent has demonstrated activity in preclinical models and early clinical trials and is currently being studied in Phase IIb trials, where efficacy and safety will be more fully assessed.

When administered orally in the form of an immediate release tablet, the Agent is released from the tablet dosage form and absorbed across the gastro-intestinal tract to provide a rapid increase in plasma concentration in a short period of time. For example, after oral administration of the immediate release formulation described in Example 1 at a dose of 5 mg to a human subject in the fasted state, the geometric mean maximum plasma concentration (Cₘₐₓ) achieved is approximately 73 ng/ml and the time at which the peak plasma concentration is observed (Tₘₐₓ) is in the range of approximately 0.25-1.5 hours (mean 0.6 hours). Following the Cₘₐₓ, the plasma concentrations of the Agent falls to less than approximately 6% of the Cₘₐₓ within 2 hours. The area under the plasma concentration-time curve from time zero up to 24 hours post-dose (AUC₀₋₂₄) is approximately 0.102 μg·hr/mL and the Cₘₐₓ/AUC₀₋₂₄ ratio is approximately 0.72.

The applicants have surprisingly found that a modified release formulation that reduces the Cₘₐₓ and also maintains a concentration level of the Agent over a prolonged period of time provides particular clinical benefits. The modified release formulations are able to provide a controlled rate of fractional uric acid excretion over an extended period of time. Particular formulations of the invention provide favourable characteristics in regards to high bioavailability and/or other pharmacokinetic behavior related to efficacy and/or safety. Such formulation characteristics are expected to result in an improved treatment option for the management of diseases or medical conditions mediated alone or in part by uric acid metabolism, including hyperuricemia, gout and many other disease states.

There is, a need for improved pharmaceutical compositions containing the Agent, particularly suitable compositions in which the Cₘₐₓ achieved by the Agent following administration is lower than achieved from an oral immediate release tablet and the concentration level is maintained over a prolonged period of time to ensure that a steady and controlled rate of fractional uric acid excretion is achieved upon dosing.

According to a first aspect of the present invention there is provided a modified release pharmaceutical composition comprising the Agent, wherein said composition, after oral administration in the fasted state to a subject in need of treatment thereof exhibits at least one of the following:
(a) produces in the subject a geometric mean maximum plasma concentration (Cₘₐₓ) of the Agent between 1 ng/ml and 50 ng/ml; and
(b) produces a ratio of Cₘₐₓ/AUC₀₋₂₄ between 0.04 and 0.4.

According to a further aspect of the present invention there is provided a modified release pharmaceutical composition comprising the Agent, wherein said composition, after oral administration in the fasted state to a subject in need of treatment thereof exhibits both of the following:
(a) produces in the subject a Cₘₐₓ of the Agent between 1 ng/ml and 40 ng/ml (conveniently between 5 ng/ml and 20 ng/ml); and
(b) produces a ratio of Cₘₐₓ/AUC₀₋₂₄ between 0.04 and 0.4.

According to a further aspect of the present invention there is provided a modified release pharmaceutical composition comprising the Agent, wherein said composition, after oral administration in the fasted state to a subject in need of treatment thereof produces in the subject a Cₘₐₓ of the Agent between 1 ng/ml and 40 ng/ml. Conveniently, the Cₘₐₓ of the Agent is between 5 ng/ml and 20 ng/ml.

According to a further aspect of the present invention there is provided a modified release pharmaceutical composition comprising the Agent, wherein said composition, after oral administration in the fasted state to a subject in need of treatment thereof exhibits a ratio of Cₘₐₓ/AUC₀₋₂₄ between 0.04 and 0.4.

According to a further aspect of the present invention there is provided a modified release pharmaceutical composition comprising the Agent, wherein said composition, after oral administration in the fasted state to a subject in need of treatment thereof exhibits a ratio of Cₘₐₓ/AUC₀₋₂₄ between 0.04 and 0.3.

According to a further aspect of the present invention there is provided a modified release pharmaceutical composition comprising the Agent, wherein said composition, after oral administration in the fasted state to a subject in need of treatment thereof exhibits a ratio of Cₘₐₓ/AUC₀₋₂₄ between 0.04 and 0.2.

According to a further aspect of the present invention there is provided a modified release pharmaceutical composition comprising the Agent, wherein said composition, after oral administration in the fasted state to a subject in need of treatment thereof exhibits a ratio of Cₘₐₓ/AUC₀₋₂₄ between 0.04 and 0.18.

According to a further aspect of the present invention there is provided a modified release pharmaceutical composition comprising the Agent, wherein said composition, after oral administration in the fasted state to a subject in need of treatment thereof exhibits a ratio of Cₘₐₓ/AUC₀₋₂₄ between 0.04 and 0.16.

According to a further aspect of the present invention there is provided a modified release pharmaceutical composition comprising the Agent, wherein said composition, after oral administration in the fasted state to a subject in need of treatment thereof exhibits a ratio of Cₘₐₓ/AUC₀₋₂₄ between 0.04 and 0.13.

According to a further aspect of the present invention there is provided a modified release pharmaceutical composition comprising the Agent, wherein said composition, after oral administration in the fasted state to a subject in need of treatment thereof exhibits a ratio of Cₘₐₓ/AUC₀₋₂₄ selected from between 0.04 and 0.4, between 0.04 and 0.3, between 0.04 and 0.2, between 0.04 and 0.18, between 0.04 and 0.16 and between 0.04 and 0.13.

According to a further aspect of the present invention there is provided a modified release pharmaceutical composition comprising the Agent, wherein said composition, after oral administration at a dose selected from within a range of 0.5-20 mg, for example 0.5, 0.67, 0.75, 0.83, 1, 1.25, 1.5, 2, 2.5, 3, 3.3, 4.5, 5, 6, 7.5, 9, 10, 12, 15 and 20 mg in the fasted state to a subject in need of treatment thereof exhibits a ratio of Cₘₐₓ/AUC₀₋₂₄ selected from between 0.04 and 0.4, between 0.04 and 0.3, between 0.04 and 0.2, between 0.04 and 0.18, between 0.04 and 0.16 and between 0.04 and 0.13. Conveniently, in this embodiment the dose is selected from 4.5, 6, 9 and 12 mg and the ratio of Cₘₐₓ/AUC₀₋₂₄ is selected from between 0.04 and 0.2, more conveniently between 0.04 and 0.16. Conveniently, the formulation is a pellet formulation.

Particular formulations of the invention are able to provide favourable characteristics, for example in regards to bioavailability and other pharmacokinetic behaviour, even in the presence of an intake of food.

Accordingly, in a particular embodiment of the present invention there is provided a modified release pharmaceutical composition comprising the Agent, wherein said composition, when orally administered after eating a meal, in comparison when administered in a fasted state, exhibits the following:
(a) produces in the subject a mean AUC and/or Cₘₐₓ, which is within 30% of the mean AUC and/or Cₘₐₓ achieved in the fasted state; and
(b) produces a ratio of Cₘₐₓ/AUC₀₋₂₄ between 0.04 and 0.4.

In a further embodiment of the present invention there is provided a modified release pharmaceutical composition comprising the Agent, wherein said composition, when orally administered after eating a meal, in comparison when administered in a fasted state, exhibits the following:
(a) produces in the subject a mean AUC and/or Cₘₐₓ, which is within 20% of the mean AUC and/or Cₘₐₓ achieved in the fasted state; and
(b) produces a ratio of Cₘₐₓ/AUC₀₋₂₄ of between 0.04 and 0.4 (conveniently between 0.04 and 0.2).

In yet a further embodiment of the present invention there is provided a modified release pharmaceutical composition comprising the Agent, wherein said composition, when orally administered after eating a meal, in comparison when administered in a fasted state, exhibits the following:
(c) produces in the subject a mean AUC and/or Cₘₐₓ, which is within 10% of the mean AUC and/or Cₘₐₓ achieved in the fasted state; and
(d) produces a ratio of Cₘₐₓ/AUC₀₋₂₄ between 0.04 and 0.3 (conveniently between 0.04 and 0.2).

Accordingly, in a particular embodiment of the present invention there is provided a modified release pharmaceutical composition comprising the Agent, wherein said composition can be administered with food with a reduced impact (conveniently a substantially reduced impact) on the release and pharmacokinetics of the Agent. In one aspect of this embodiment, there is provided a modified release pharmaceutical composition comprising the Agent, wherein said composition can be administered with food with a minimal impact on release and pharmacokinetics of the Agent.

In one embodiment, particular formulations of the invention provide favorable characteristics in regards to pharmacokinetic behavior and a related reduction of adverse effects.

In one embodiment of the present invention there is provided a modified release pharmaceutical composition comprising the Agent, wherein said composition, when orally administered in the fasted state to a subject in need of treatment thereof, maintains a plasma concentration at 2 hours post Tₘₐₓ that is at least 15% of the Cₘₐₓ. Conveniently, the plasma concentration at 2 hours post Tₘₐₓ is at least 30% (more conveniently 40%, and yet more conveniently 50%) of the Cₘₐₓ.

In one embodiment of the present invention there is provided a modified release pharmaceutical composition comprising the Agent, wherein after oral administration at a dose in the range of 0.5-5 mg (conveniently 4.5 mg) in the fasted state to a subject in need of treatment thereof produces a AUC₀₋₂₄ of about 35 ng·hr/mL or more, conveniently 45 ng·hr/mL or more, yet more conveniently 70 ng·hr/mL or more.

In one embodiment of the present invention there is provided a modified release pharmaceutical composition comprising the Agent, wherein after oral administration at a dose of 5 mg in the fasted state to a subject in need of treatment thereof produces a AUC₀₋₂₄ of about 35 ng·hr/mL or more, conveniently 45 ng·hr/mL or more, yet more conveniently 70 ng·hr/mL or more.

In one embodiment of the present invention there is provided a modified release pharmaceutical composition comprising the Agent, wherein after oral administration at a dose in the range of 5-30 mg (conveniently 6 or 12 mg) in the fasted state to a subject in need of treatment thereof produces a AUC₀₋₂₄ of about 100 ng·hr/mL or more, conveniently 120 ng·hr/mL or more, yet more conveniently 140 ng·hr/mL or more.

In one embodiment of the present invention there is provided a modified release pharmaceutical composition comprising the Agent, wherein after oral administration at a dose of 10 mg in the fasted state to a subject in need of treatment thereof produces a AUC₀₋₂₄ of about 100 ng·hr/mL or more, conveniently 120 ng·hr/mL or more, yet more conveniently 140 ng·hr/mL or more.

As used herein and unless stated otherwise, it is to be understood that the term “about” is used synonymously with the term “approximately”. Illustratively and unless stated otherwise, the use of the term “about” indicates values slightly outside the cited criteria values, namely, ±10% (conveniently ±2%). Such values are thus encompassed by the scope of the claims reciting the terms “about” or a pproximately”.

As used herein, the term “immediate release” or “IR” is used in its conventional sense to refer to a dosage form that provides for release of the Agent immediately after administration. For example, an immediate release formulation means a formulation in which the dissolution rate of the drug from the formulation is 85% or more after 30 minutes from the beginning a dissolution test, which is carried out in accordance with a dissolution test (paddle method) described in the United States Pharmacopoeia under the conditions that 900 mL of an appropriate test fluid (such as a USP buffer, pH 6.8) is used and the paddle rotation speed is 100 rpm. Alternatively, the term means a formulation in which the dissolution rate of the drug from the formulation is 85% or more after 30 minutes from the beginning a dissolution test, which is carried out in accordance with a dissolution test, method 2 (paddle method) described in the Japanese Pharmacopoeia under the conditions that 900 mL of a USP phosphate buffer (pH 6.8) is used as a test fluid and the paddle rotation speed is 200 rpm.

As used herein, the term “modified release” or “MR” means that the escape or release of a drug, such as the Agent, from the dosage form (tablet, capsule, pellet, etc.) has been modified so that the release rate is slower than that from an unmodified or immediate release dosage form. Drug release may occur over several hours or over several days in order to maintain a therapeutically effective plasma concentration of the drug. Modified release encompasses delayed release (release at a time other than immediately after administration), extended release (release over a prolonged time period), sustained release (rate of drug release is sustained over a period of time), and controlled release (rate of drug release is controlled to get a particular drug concentration profile in the body). As used herein, a slower dissolution profile is one in which the escape or release of a drug from the dosage form is slower, i.e. it takes more time for the drug to be released in a slower dissolution profile than a faster dissolution profile. Conveniently, the modified release is extended release, sustained release or controlled release.

### The Agent

The solubility of 2-((3-(4-cyanonaphthalen-1-yl)pyridine-4-yl)thio)-2-methylpropanoic acid in aqueous media is highly dependent upon pH. The following table shows the aqueous equilibrium solubility of the compound measured at 37° C.:

Solubility of 2-((3-(4-cyanonaphthalen-1-yl)pyridine-
4-yl)thio)-2-methylpropanoic acid at 37° C.

| | pH | Solubility after | pH | Solubility after | pH |
|---|---|---|---|---|---|
| Vehicle | initial | 24 hours (mg/mL) | (24 h) | 48 hours (mg/mL) | (48 h) |
| 0.1N HCl | 1.1 | 1.480 | 1.0 | 1.238 | 1.1 |
| SGF pH 1.6 | 1.6 | 0.415 | 1.4 | 0.398 | 1.4 |
| Citrate pH 3.0 | 3.0 | 0.066 | 2.8 | 0.065 | 2.8 |
| Succinate pH 4.0 | 4.0 | 0.056 | 3.9 | 0.055 | 3.9 |
| Acetate pH 4.5 | 4.6 | 0.070 | 4.5 | 0.065 | 4.5 |
| Citrate pH 5.0 | 5.0 | 0.097 | 5.0 | 0.095 | 4.9 |
| Histidine pH 6.0 | 6.1 | 0.532 | 5.8 | 0.485 | 5.8 |
| Potassium phosphate pH 6.8 | 6.9 | 2.370 | 6.6 | 2.707 | 6.5 |
| Sodium phosphate pH 7.0 | 7.0 | 3.524 | 6.7 | 3.620 | 6.6 |
| 0.03N NaOH | 12.3 | 10.562 | 6.9 | 10.256 | 6.9 |
| 0.1N NaOH | 12.8 | 31.871 | 7.1 | 33.494 | 7.0 |
| 0.3N NaOH | 13.2 | 87.766 | 11.7 | 93.616 | 7.4 |
| Water | 6.3 | 0.148 | 5.4 | 0.132 | 5.3 |

The Agent may be used in the free form or as a pharmaceutically acceptable salt, such as a pharmaceutically acceptable basic addition salt formed through reaction with a suitable base, such as the hydroxide, carbonate, bicarbonate, sulphate, of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminium salts and the like. Illustrative examples of bases include sodium hydroxide, potassium hydroxide, choline hydroxide, sodium carbonate, N⁺(C₁₋₄ alkyl)₄, and the like. Representative organic amines useful for the formation of base addition salts include ethylamine, diethylamine, ethylenediamine, ethanolamine, diethanolamine, piperazine and the like.

The Agent may be used as a pharmaceutically acceptable salt, such as a pharmaceutically acceptable acid addition salt formed through reaction with a suitable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, Q-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid and muconic acid.

Conveniently, the Agent is used in the free form.

The Agent may be used in various solid state forms, all of which are included within the scope of the invention. These include amorphous or crystalline forms, and anhydrous forms as well as solvates or hydrates. In a particular group of formulations, the Agent is crystalline and is in the anhydrous form.

It is further to be understood that the Agent could be used in the form of a suitable pharmaceutically-acceptable pro-drug. Accordingly, the Agent may be administered in the form of a pro-drug that is a compound that is broken down in the human or animal body to release the Agent. The term “prodrug” as used herein, refers to a drug precursor that, following administration to an individual and subsequent absorption, is converted to an active, or a more active species via some process, such as conversion by a metabolic pathway. Thus, the term encompasses any derivative of the Agent, which, upon administration to a recipient, is capable of providing, either directly or indirectly, the Agent or a pharmaceutically active metabolite or residue thereof. Some prodrugs have a chemical group present on the prodrug that renders it less active and/or confers solubility or some other property to the drug. Once the chemical group has been cleaved and/or modified from the prodrug the active drug is generated. Prodrugs can be useful because, in some situations, they may be easier to administer than the parent drug or may have other benefits for example where delivery of a drug to specific area of the body is required.

The dose of Agent required in the composition of the invention for the therapeutic or prophylactic treatment of a particular disease or medical condition will necessarily be varied depending on for example, the host treated and the severity of the illness being treated. The amount of the active compound administered will be dependent on the subject being treated, the severity of the disorder or condition, the rate of administration, the disposition of the compound and the discretion of the prescribing physician. However, an effective dosage is in the range of about 0.003 to about 10 mg per kg body weight per day, preferably about 0.003 to about 1 mg/kg/day, in single or divided doses. For a 70 kg human, this would amount to about 0.21 to 700 mg/day, preferably about 0.21 to about 70 mg/day. In some instances, dosage levels below the lower limit of the aforesaid range may be more than adequate, while in other cases still larger doses may be employed without causing any harmful side effect, provided that such larger doses are first divided into several small doses for administration throughout the day. A unit dose of the composition will usually contain, for example 0.1-100 mg of active ingredient, and preferably 0.2-10 mg of active ingredient. Preferably a daily dose selected from any of the following is envisaged, 0.5 mg, 1 mg, 1.5 mg, 2 mg, 2.5 mg, 3 mg, 3.5 mg, 4.0 mg, 4.5 mg, 5 mg, 10 mg, 12.5 mg, 15 mg and 20 mg. It will be understood that a broad range of doses is considered to account for the diverse needs of the clinical population which may show differences in exposure as well as differences in exposure from different formulations.

Typically the Agent will be present in the composition of the invention in an amount within the range of from 0.5 to 50%, suitably from about 0.5 to 35% and especially from about 0.5 to 30% by weight of the composition. It is to be understood that the term ‘about’ when relating to the proportion of Agent present in the composition refers to ±2% by weight of the total composition.

### Modified Release Dosage Forms

The benefits of the present invention are not limited to a particular type of dosage form having a particular mechanism of drug release. Conveniently, the modified release compositions of the invention provides release of the Agent over a period of 3 hours or longer, conveniently 4 hours of longer, more conveniently 5 hour or longer, yet more conveniently 8 hours or longer, yet more conveniently 12 hours or longer, yet more conveniently 15 hours or longer, post administration. Release of the Agent can be determined by methods known in the art. For example, release rates can be determined using in-vitro dissolution tests as described in the Examples herein.

Modified release of the Agent may be accomplished by any means known in the pharmaceutical art, including but not limited to the use of osmotic dosage forms, matrix dosage forms, multiparticulate dosage forms, gastric retentive dosage forms, and pulsatile dosage forms. Two of these examples, namely matrix dosage forms and multiparticulate dosage forms, are described in greater detail below.

### Matrix Systems (Single Unit Dosage Forms)

In one embodiment, the Agent is incorporated into an erodible or non-erodible matrix modified release dosage form. Typically, in a matrix dosage form the drug is homogenously dispersed in a matrix material. By erodible matrix is meant aqueous-erodible or water-swellable or aqueous-soluble in the sense of being either erodible or swellable or dissolvable in pure water or requiring the presence of an acid or base to ionize the polymeric matrix sufficiently to cause erosion or dissolution. When contacted with an aqueous environment, the erodible matrix imbibes water and forms an aqueous-swollen gel or “matrix” that the Agent can pass or diffuse through depending on its physicochemical properties. The aqueous-swollen matrix gradually erodes, swells, disintegrates or dissolves, thereby controlling the release of the Agent. The erodible matrix into which the Agent is incorporated may generally be described as a set of excipients that are mixed with the Agent that, when contacted with the aqueous environment imbibes water and forms an aqueous-swollen gel or “matrix” that entraps the Agent. Drug release may occur by a variety of mechanisms: the matrix may disintegrate or dissolve from around particles or granules of the Agent; or the drug may dissolve in the imbibed aqueous solution and diffuse from or through the matrix dosage form.

A key ingredient of this water-swollen matrix is the water-swellable, erodible, or soluble polymer, which may typically be described as a hydrogel or water-swellable polymer. Such polymers may be linear, branched, or crosslinked. They may be homo-polymers or co-polymers. Although they may be synthetic polymers derived from vinyl, acrylate, methacrylate, urethane, ester and oxide monomers, they are most conveniently derivatives of naturally occurring polymers such as polysaccharides or proteins. Such materials include naturally occurring polysaccharides such as chitin, chitosan, dextran and pullulan; gum agar, gum arabic, gum karaya, locust bean gum, gum tragacanth, carrageenans, gum ghatti, guar gum, xanthan gum and scleroglucan; starches such as dextrin and maltodextrin; hydrophilic colloids such as pectin; phosphatides such as lecithin; alginates such as ammonium alginate, sodium, potassium or calcium alginate, propylene glycol alginate; gelatin; collagen; and cellulosics. By “cellulosics” is meant a cellulose polymer that has been modified by reaction of at least a portion of the hydroxyl groups on the saccharide repeat units with a compound to form an ester-linked or an ether-linked substituent.

A preferred class of cellulosics for the erodible matrix comprises cellulosics such as ethyl cellulose (EC), methylethyl cellulose (MEC), carboxymethyl cellulose (CMC), carboxymethyl ethyl cellulose (CMEC), hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose or hypromellose (HPMC), HPMCP, HPMCAS, hydroxypropyl methyl cellulose acetate trimellitate (HPMCAT), and ethylhydroxy ethylcellulose (EHEC). A particularly convenient class of such cellulosics comprises various grades of low viscosity (MW less than or equal to 50,000 daltons) and high viscosity (MW greater than 50,000 daltons) HPMC.

The HPMC may contain more than one grade of polymer and is commercially available under several trademarks, e.g. METHOCEL® E, F, J and K from the Dow Chemical Company, USA. Commercially available low viscosity HPMC polymers include the Dow METHOCEL series E5, E15LV, E50LV and K100LY, while high viscosity HPMC polymers include E4MCR, E10MCR, K4M, K15M and K100M; especially preferred in this group are the METHOCEL (Trademark) K series. Conveniently the HPMC is METHOCEL K100 Premium LVCR or METHOCEL K100M Premium DC. Other commercially available types of HPMC include the Shin Etsu METOLOSE 90SH series and the Ashland Benecal™ series.

Other materials useful for the erodible matrix material include, but are not limited to, polyethylene oxide, pullulan, polyvinyl pyrrolidone, polyvinyl alcohol, polyvinyl acetate, glyceryl fatty acid esters, polyacrylamide, polyacrylic acid, copolymers of ethacrylic acid or methacrylic acid (EUDRAGIT®, Rohm America, Inc., Piscataway, N.J.) and other acrylic acid derivatives such as homopolymers and copolymers of butylmethacrylate, methylmethacrylate, ethylmethacrylate, ethylacrylate, (2-dimethylaminoethyl)methacrylate, and (trimethylaminoethyl) methacrylate chloride.

In one embodiment, the erodible matrix material is polyethylene oxide. Examples include product names, Polyox WSR-308 [average molecular weight: 8,000,000, viscosity: 10,000-15,000 mPa·s (1% aqueous solution at 25° C.)], Polyox WSR-303 [average molecular weight: 7,000,000, viscosity: 7,500-10,000 mPa·s (1% aqueous solution at 25° C.)], Polyox WSR Coagulant [average molecular weight: 5,000,000, viscosity: 5,500-7,500 mPa·s (1% aqueous solution at 25° C.)], 5 Polyox WSR-301 [average molecular weight: 4,000,000, viscosity: 1,650-5,500 mPa·s (1% aqueous solution at 25° C.)], Polyox WSR-N-60K [average molecular weight: 2,000,000, viscosity: 2,000-4,000 mPa·s (2% aqueous solution at 25° C.)], Polyox WSR-N-12K [average molecular weight: 1,000,000, viscosity: 400-800 mPa·s (2% aqueous solution at 25° C.)], Polyox WSR-1105 (average molecular weight: 900,000, viscosity: 8,800-17,600 mPa·s (5% aqueous solution at 25° C.)], Polyox WSR-205 [average molecular weight: 600,000, viscosity: 4,500-8,800 mPa·s (5% aqueous solution at 25° C.)], Polyox WSR-N-750 [average molecular weight: 300,000, viscosity: 600-1200 mPa·s (5% aqueous solution at 25° C.)], Polyox WSR-N-80 [average molecular weight: 200,000, viscosity: 55-90 mPa·s (5% aqueous solution at 25° C.)], and Polyox WSR-N-10 [average molecular weight: 100,000, viscosity: 12-50 mPa·s (5% aqueous solution at 25° C.)] (the Dow Chemical Company, USA). Conveniently, the polyethylene oxide is Polyox WSR-N-750.

These erodible matrix polymers may be used alone, or as an appropriate combination of two or more thereof. The erodible matrix polymer(s) will, in general, be present in about 5 to 50% by weight of the composition, conveniently about 5 to 40% by weight, more conveniently about 5 to 35% by weight and yet more conveniently about 5 to 30% by weight. In one embodiment, the erodible matrix polymer is hydroxypropyl methylcellulose and is present in about 10 to 35% by weight of the composition, conveniently about 17.5 to 30% by weight, more conveniently about 18-22% (conveniently 19%) or about 25-32% (conveniently 29%) by weight, yet more conveniently 19.42% or 29.13% by weight. Conveniently, the hydroxypropyl methyl cellulose is a low viscosity (MW less than or equal to 50,000 daltons) or high viscosity (MW greater than 50,000 daltons) HPMC. Conveniently, the HPMC is selected from the METHOCEL K100 Premium LVCR or METHOCEL K100M. Conveniently, the HPMC is METHOCEL K100M Premium DC. In a further embodiment, both hydroxypropyl methyl cellulose and polyethylene oxide are present as erodible matrix polymers, wherein the hydroxypropyl methylcellulose is present in about 10 to 20% by weight of the composition (conveniently about 15%) and the polyethylene oxide is present in about 5 to 10% by weight of the composition (conveniently about 9-10%). Conveniently, the polyethylene oxide is Polyox WSR-N-750.

The erodible matrix polymer composition may additionally contain a wide variety of pharmaceutically acceptable excipients known in the pharmaceutical arts, including excipients that ease the manufacturing process and/or improve the performance of the dosage form. Common excipients include diluents or bulking agents, lubricants, binders, etc. Such additional excipients are well known to those skilled in the art and are described in, for example the Handbook of Pharmaceutical Excipients, 7th Edition, American Pharmaceutical Association; The Theory and Practice of Industrial Pharmacy, 4rd Edition, Khar et al. 2013; Pharmaceutical Dosage Forms: Tablets Volume 1, 3rd Edition, Augsburger., et al, 2008; Modern Pharmaceutics, Banker, Gilbert and Rhodes, Christopher T, 4th edition, 2002; and Remington: The Science and Practice of Pharmacy, 22nd Edition, 2012.

The amount of excipients used in the dosage form will correspond to those typically used in a matrix system. The excipient(s) will, in general, be present in about 10 to 90% by weight of the composition, conveniently about 20 to 90% by weight, more conveniently about 40 to 90% by weight, most conveniently about 60 to 80% by weight, yet most conveniently about 63 to 80% by weight and especially about 66 to 79% by weight.

Diluents, or fillers, can be added in order to increase the mass to a size suitable for tablet compression containing an individual dose. Suitable diluents include powdered sugar, calcium phosphate, calcium sulphate, microcrystalline cellulose, lactose, mannitol, kaolin, sodium chloride, starch and sorbitol. Diluents or fillers can be present in about 20-85% by weight of the composition, conveniently about 45-80% by weight, more conveniently about 60 to 75% by weight. Conveniently, the diluent is microcrystalline cellulose or lactose. In one embodiment, the diluent is microcrystalline cellulose and is present in 61-65% by weight of the composition. In a further embodiment, both microcrystalline cellulose and lactose are present, wherein the microcrystalline cellulose is present in 45-50% by weight of the composition and the lactose is present in 22-25% by weight of the composition.

Lubricants can be incorporated into the dosage form for a variety of reasons. Lubricants reduce friction between the granulation and die wall during compression and ejection. This prevents the granulate from sticking to the tablet punches and facilitates its ejection from the tablet punches. Examples of suitable lubricants that can be used include, but are not limited to, talc, stearic acid, palmitic acid, vegetable oil, sodium stearyl fumarate, calcium stearate, zinc stearate and magnesium stearate. Lubricants can be present in about 0.1-4% by weight of the composition, conveniently about 0.2-1% by weight, more conveniently about 0.2 to 0.75% by weight. Conveniently, the lubricant is magnesium stearate.

Glidants can also be incorporated into the dosage form. A glidant improves the flow characteristics of the granulation. Examples of suitable glidant's include, but are not limited to, talc, silicon dioxide and starch. Glidants can be present in about 0.1-0.75% by weight of the composition, conveniently about 0.2-0.5% by weight. Conveniently, the glidant is colloidal silicon dioxide.

Binders can be incorporated into the dosage form. Binders are typically utilized if the manufacture of the dosage form includes a granulation step. Examples of suitable binders include, but are not limited to, povidone, polyvinylpyrrolidone, xanthan gum, cellulose gums such as carboxymethylcellulose, methylcellulose, hydroxyethyl cellulose, hydroxypropyl cellulose, hydroxypropyl methylcellulose (hypromellose), hydroxycellulose, gelatin, starch, and pregelatinized starch.

Other excipients that can be incorporated into the dosage form include, but are not limited to, pH modifiers (such as suitable organic acids or alkali metals (e.g. lithium, sodium or potassium) salts thereof, such as benzoic acid, citric acid, tartaric acid, succinic acid, adipic acid and the like or the corresponding alkali metal salts thereof, for example the alkali metal salts of such acids, e.g. the sodium salt of citric acid (i.e. sodium citrate)). Other excipients that could be present include, but are not limited to, preservatives, antioxidants, or any other excipient commonly used in the pharmaceutical industry.

In one embodiment, typically the Agent will be present in the matrix composition of the invention in an amount within the range of from 0.5 to 50%, suitably from about 0.5 to 20% and especially from about 1 to 10% by weight of the composition. In a particular group of compositions, the Agent will be present in an amount of about 2-3% by weight of the final composition. In a further particular group of compositions, the Agent will be present in an amount of about 5-6% by weight of the final composition. In yet a further particular group of compositions, the Agent will be present in an amount of about 2 or 3% by weight of the final composition. In yet a further particular group of compositions, the Agent will be present in an amount of about 5 or 6% by weight of the final composition. In yet a further particular group of compositions, the Agent will be present in an amount of 2-3%, such as for example 2.31 or 2.43%, by weight of the final composition. In yet a further particular group of compositions, the Agent will be present in an amount of 5-6%, such as for example 5.39%, by weight of the final composition.

Alternatively, the compositions of the present invention may be administered by or incorporated into a non-erodible matrix dosage form. In such dosage forms, the Agent is distributed in an inert matrix. The drug is predominantly released by diffusion through the inert matrix. Examples of materials suitable for the inert matrix include insoluble plastics, such as methyl acrylate-methyl methacrylate copolymers, polyvinyl chloride, and polyethylene; polymers, such as ethyl cellulose, cellulose acetate, and crosslinked polyvinylpyrrolidone (also known as polyvinylpolypyrrolidone or crospovidone); and fatty compounds, such as carnauba wax, microcrystalline wax, and triglycerides. Such dosage forms are described further in Remington: The Science and Practice of Pharmacy 22^{nd} edition (2012).

Matrix controlled release dosage forms may be prepared by blending the Agent and other excipients together, and then forming the blend into a tablet (e.g. a caplet), pill, or other dosage form, for example by compressive forces. The formulations of the invention may, for example, be prepared by technology such as wet granulation, direct compression, dry compaction (e.g. roller compaction) and the like. For example, they can be prepared by blending the matrix polymer with the Agent and optionally other excipients followed by granulating the mixture before compressing the mixture into the final dosage form. Such compressed dosage forms may be formed using any of a wide variety of presses used in the fabrication of pharmaceutical dosage forms. Examples include single-punch presses, rotary tablet presses, and multilayer rotary tablet presses. See for example, Remington: The Science and Practice of Pharmacy, Edition, 22^{nd} Edition, 2012. The compressed dosage form may be of any shape, including round, oval, oblong, cylindrical, or triangular. The upper and lower surfaces of the compressed device may be flat, round, concave, or convex. When formed by compression, the dosage form conveniently has a “strength” of at least 5 kiloponds (kp)/cm², and more preferably at least 7 kp/cm². Here, “strength” is the fracture force, also known as the tablet “hardness,” required to fracture a tablet formed from the materials, divided by the maximum cross-sectional area of the tablet normal to that force. The compression force required to achieve this strength will depend on various factors such as for example, the size of the tablet, but generally the strength will be greater than about 5 kp/cm². Friability is a well-known measure of a tablet's resistance to surface abrasion by weight loss in percentage after subjecting the tablet to a standardized agitation procedure. Friability values of from 0.8 to 1.0% are regarded as constituting the upper limit of acceptability. Devices having a strength of greater than 5 kp/cm² (dependant on size) generally are very robust, having a friability of less than 0.5%.

Conveniently, a wet granulation process for preparing matrix controlled dosage formulations of the invention comprises the following steps:
(a) mixing the Agent, a matrix material and optionally other excipients;
(b) wet granulating the mixed components;
(c) drying the mixture;
(d) blending the mixture with a lubricant such as magnesium stearate and optionally adding other excipients; and
(e) compressing the blended mixture into tablets.

Other methods for forming matrix controlled-release formulations are well known in the pharmaceutical arts. See for example, Remington: The Science and Practice of Pharmacy Edition, 2000, 22^{nd} Edition, 2012.

The matrix dosage forms may optionally be coated with one or more suitable coatings, for example a film coating. A coating can be used to aid ease of swallowing, ease handling, provide aesthetic properties or protection against, for example, moisture ingress or degradation by light, to colour the formulation, or to modify or control the release of the Agent from the formulation, for example to provide acid enteric protection or other release-controlling purposes.

Suitable coatings, such as film coatings, that may be applied to the composition according to the invention comprise a film-forming agent, for example a sugar or more particularly a film-forming polymer. Suitable sugar coatings are well known and comprise for example sucrose or lactose. Suitable film-forming agents include, for example film-forming polymers, such as cellulose ethers, esters and mixed ethers and esters, including, but not limited to, esters of water-soluble cellulose ethers, for example hydroxypropyl methylcellulose (hypromellose), hydroxypropyl ethylcellulose, hydroxypropylcellulose, methylcellulose, hydroxypropyl methylcellulose acetate succinate or hydroxypropyl methylcellulose phthalate; film-forming acrylic polymers, for example methacrylate-methylmethacrylate copolymers; and film-forming vinyl polymers, for example polyvinyl alcohols or polyvinyl acetate phthalate. Suitably the film-forming polymer is a water-soluble film-forming polymer, particularly a water-soluble cellulose ether for example hydroxypropyl methylcellulose-hypromellose (particularly hydroxypropyl methylcellulose with a dynamic viscosity of from 2 to 18 cP (measured in a 2% w/v solution at 20° C.) and selected from, for example grades 1828, 2208, 2906 and preferably 2910 as defined hereinbefore). The amount of film-forming agent used will depend upon the desired properties of the film coating. Generally the film forming agent will be present in an amount of from 40 to 90% by weight of the film coating, for example from 50 to 80% of the film coating. The film-forming agent is typically present at from 0.5 to 5%, suitably from 1 to 3% by weight of the composition according to the invention. Optionally the film coating contains additional components such as plasticiser, colorants, dispersion aids and opacifiers. Plasticisers may be used to improve film flexibility and durability and adhesion properties of the film coating. Suitable plasticisers include, for example glycerin, acetylated monoglycerides, citrate esters (for example triethyl citrate), propylene glycols, polyethylene glycols (for example polyethylene glycols with a molecular weight of from 200 to 500, particularly 300), triacetin (glycerol tri-acetate), triglycerides (for example castor oil), or phthalate esters (for example diethylphthalate). Generally the plasticiser, when used, is present in an amount of from 1 to 20%, for example 5 to 15% by weight based upon the weight of the film coating.

Suitable opacifiers and colorants are well known and include for example titanium dioxide, ferric oxides (for example iron oxide). Suitable dispersion aids include, for example talc.

In an embodiment of the invention the film coating comprises:
(i) from 50 to 100 (suitably from 50 to 80 parts of a water-soluble cellulose ether (suitably hydroxypropyl methylcellulose, particularly hydroxypropyl methylcellulose with a dynamic viscosity of from 2 to 18 cP (measured in a 2% w/v solution at 20° C.), for example grades 2910, 1828, 2208 or 2906 as defined hereinbefore with a dynamic viscosity of from 5 to 7 cP);
(ii) from 0 to 25 (particularly from 5 to 20) parts plasticiser (suitably polyethylene glycol, particularly polyethylene glycol with a molecular weight of from 200 to 500); and
(iii) from 0 to 50 (particularly from 0 to 30) parts in total of opacifiers (suitably titanium dioxide), colorants (suitably an iron oxide) and dispersion aids; wherein all parts are by weight and the sum of the parts (i)+(ii)+(iii)=100.

The coating may comprise, for example, 0.5 to 10% by weight of the composition, particularly 1 to 6%, and preferably 2 to 3%. Suitable film coatings are commercially available as concentrates that may be diluted with water and optionally a cellulose ether such as HPMC and a plasticiser such as polyethylene glycol prior to application to the composition. Such concentrates include Opadry® coatings ex Colorcon, for example Opadry Blue 03K105000 and Opadry White 03K18416.

In one embodiment, the matrix dosage forms are coated with one or more suitable coatings to further modify or control the release of the Agent from the formulation, for example to provide acid enteric protection or other release-controlling purposes. Suitable materials useful for preparing the coating on the matrix dosage forms include polymers known in the art as enteric coatings for delayed-release of pharmaceuticals. These most commonly are pH-sensitive materials such as cellulose acetate phthalate, cellulose acetate trimellitate, hydroxypropyl methyl, cellulose phthalate, poly(vinyl acetate phthalate), and acrylic copolymers such as Eudragit L-100 (Evonik), Eudragit L 30 D-55, Eudragit S 100, Eudragit FS 300, and related materials. Conveniently the coating material is Eudragit L 30 D-55. The coating material is typically present at from 0.5 to 7%, suitably from 1 to 5% by weight of the composition according to the invention. The thickness and type of the delayed-release coating is adjusted to give the desired delay property. In general, thicker coatings are more resistant to erosion and, consequently, yield a longer delay as do coatings which are designed to dissolve above pH 7. Preferred coatings typically range from about 10 micron in thickness to about 3 mm in thickness and more preferably 10 um to 500 um. When ingested, the matrix dosage form passes through the stomach, where the coating prevents release of the Agent under the acidic conditions prevalent there. When the matrix dosage form passes out of the stomach and into the small intestine, where the pH is higher, the coating erodes or dissolves according to the physicochemical properties of the chosen material. Upon erosion or dissolution of the coating, the erodible or non-erodible matrix that the Agent is incorporated into prevents immediate or rapid release of the Agent and modulates the release so as to prevent the production of high concentrations.

In a particular embodiment there is provided a matrix modified release pharmaceutical composition comprising:
(i) from 1 to 10 (particularly from 1 to 8) parts of the Agent;
(ii) from 10 to 40 (particularly from 15 to 35) parts of an the erodible matrix polymer;
(iii) from 40 to 85 (particularly from 40 to 75) parts of a diluent or a combination of diluents;
(iv) from 0 to 3 (particularly from 0.2-0.7) parts of a glidant;
(v) from 0 to 2 (particularly from 0.2-1) parts of a lubricant; and
(vi) from 0 to 8 (particularly from 0.5-3.5) parts of a film coating;
   wherein all parts are by weight and the sum of the parts (i)+(ii)+(iii)+(iv)+(v)+(vi)=100, the erodible matrix polymer has any of the meanings defined hereinbefore.

### Multiparticulate Systems

Multiparticulate systems include subunits such as mini-tablets, beads, pellets, and granules. Multiparticulates generally comprise a plurality of minitablets, beads, pellets or granules that may range in size from about 10 μm to about 2 mm, more typically about 100 μm to 1 mm in diameter. Such multiparticulates may be packaged, for example, in a capsule such as a gelatin capsule or a capsule formed from a polymer such as HPMCAS, HPMC or starch; dosed as a suspension or slurry in a liquid; dosed in a sachet; or they may be formed into a tablet (e.g. a caplet) or pill by compression or other processes known in the art.

Such multiparticulates may be made by any known process, such as wet- and dry-granulation processes, extrusion/spheronization, roller-compaction, melt-congealing, or by spray-coating seed cores. Conveniently, the multiparticulates are made by spray-coating seed cores.

For example, in wet- and dry-granulation processes, the composition comprising the Agent and optional excipients may be granulated to form multiparticulates of the desired size. Excipients, such as a binder, may be blended with the composition to aid in processing and forming the multiparticulates. Binders useful in fabrication of multiparticulates include microcrystalline cellulose (e.g., Avicel®, FMC Corp.), hydroxypropyl cellulose (HPC), hydroxypropyl methyl cellulose (HPMC), and related materials or combinations thereof. In general, binders which are useful in granulation and tabletting, such as starch, pregelatinized starch, and poly (N-vinyl-2-pyrrolidinone) (PVP) may also be used to form multiparticulates. In the case of wet granulation, a binder such as microcrystalline cellulose may be included in the granulation fluid to aid in forming a suitable multiparticulate. See, for example, Remington: The Science and Practice of Pharmacy, 22^{nd} Edition, 2012.

In any case, the resulting particles may themselves constitute the multiparticulate or they may be coated by various film-forming materials such as modified release polymers, enteric polymers or water-swellable or water-soluble polymers, and/or they may be combined with other excipients or vehicles to aid in dosing to patients. Conveniently, the resulting particles are coated by modified release polymers, and/or they may be combined with other excipients or vehicles to aid in dosing to patients.

In one embodiment, the Agent is present within a core surrounded by a rate-limiting membrane. The Agent traverses the membrane by mass transport mechanisms, including but not limited to dissolution in the membrane followed by diffusion across the membrane or diffusion through liquid-filled pores within the membrane. Each subunit of the multiparticulate can be individually coated with a membrane. The coating can be non-porous, yet permeable to the Agent (for example the Agent may diffuse directly through the membrane), or it may be porous. Modified release coatings as known in the art may be employed to fabricate the membrane, especially polymer coatings, such as a cellulose ester or ether, an acrylic polymer, or a mixture of polymers. Preferred materials include ethyl cellulose, cellulose acetate and cellulose acetate butyrate. The polymer may be applied as a solution in an organic solvent or as an aqueous dispersion or latex. The coating operation may be conducted in standard equipment such as a fluid bed coater, a Wurster coater, or a rotary bed coater. If desired, the permeability of the coating may be adjusted by blending of two or more materials. A useful process for tailoring the porosity of the coating comprises adding a pre-determined amount of a finely-divided water-soluble material, such as sugars or salts or water-soluble polymers (e.g. HPC) to a solution or dispersion (e.g., an aqueous latex) of the membrane-forming polymer to be used. When the dosage form is ingested into the aqueous medium of the GI tract, these water soluble membrane additives are leached out of the membrane, leaving pores which facilitate release of the drug. The membrane coating can also be modified by the addition of plasticizers, as known in the art.

In one preferred embodiment, the multiparticulate comprises a seed core layered with the Agent and coated with a polymeric material of the type useful for providing modified release of the Agent. In this embodiment, multiparticulates in the form of beads or pellets may be prepared by building the Agent composition (drug plus optionally any excipients) up on a seed core by a drug-layering technique such as powder coating or by applying the Agent composition by spraying a solution or dispersion of the Agent in an appropriate solution/dispersion vehicle (e.g. a binder dispersion, for example HPMC, e.g. 6 cps) onto seed cores in a fluidized bed such as a Wurster coater or a rotary processor. The seed core can be comprised of a sugar (for example a non-pareil seed), starch or microcrystalline cellulose, conveniently microcrystalline cellulose. An example of a suitable composition and method is to spray a dispersion of the Agent/binder (e.g. HPMC) composition in water on to the seed core. A modified release coating as known in the art and as previously described, especially polymer coatings, may be employed to fabricate the membrane, which is applied over the Agent layered seed cores. The rate of Agent release from the coated multiparticulates can be controlled by factors such as the composition and binder content of the Agent-coated core, the thickness and permeability of the modified release coating, and the surface-to-volume ratio and size of the multiparticulates. It will be appreciated by those skilled in the art that increasing the thickness of the coating will decrease the release rate, whereas increasing the permeability of the coating or the size or surface-to-volume ratio of the multiparticulates will increase the release rate. If desired, the permeability of the coating may be adjusted by blending of two or more materials. A useful series of modified release coatings comprises mixtures of water-insoluble and water-soluble polymers, for example, ethylcellulose and hydroxypropylcellulose, respectively. A useful modification to the coating is the addition of finely-divided water-soluble material, such as sugars or salts. When placed in an aqueous medium, these water soluble membrane additives are leached out of the membrane, leaving pores which facilitate delivery of the drug. The membrane coating may also be modified by the addition of plasticizers, as is known to those skilled in the art.

In one embodiment, the modified release pellets comprise:
a) an inert core in an amount ranging from about 10% to about 90% (w/w) of the weight of the modified release pellet;
b) a drug layer that encapsulates the inert core comprising a mixture of the Agent and optionally a binder (such as for example hydroxypropyl methylcellulose) in an amount ranging from about 5% to about 80% (w/w) of the total weight of the modified release pellet, the weight ratio of the Agent to the binder (when present) ranging from about 4:1 to 19:1;
c) a modified release layer that encapsulates the drug layered core comprising a modified release polymer, said modified release polymer comprising ethylcellulose or a mixture of ethylcellulose and/or hydroxypropyl cellulose in an amount ranging from about 5% to about 50% (w/w) of the total weight of the modified release pellet, the weight ratio of ethylcellulose to hydroxypropyl cellulose (when present) ranging from about 1:1 to 4:1; and
d) optionally, additional excipients, for example a lubricant such as magnesium stearate and/or a plasticizer, such as for example, triethyl citrate (TEC) or Acetate tri ethyl citrate (ATEC), at about 0.1% to about 5% (w/w) of the total weight of the modified release pellet.

In one aspect of this embodiment, a sub-coat can be applied between the drug layer and the modified release layer if separation is needed. In one aspect of this embodiment, the coat can be comprised of HPMC or magnesium stearate.

In a further embodiment, the modified release pellets comprise:
a) an inert core in an amount ranging from about 40% to about 60% (w/w) (conveniently between 50-60%, such as for example 51.3% or 58.98%) of the weight of the modified release pellet;
b) a drug layer that encapsulates the inert core comprising a mixture of the Agent and optionally a binder (such as for example hydroxypropyl methylcellulose) in an amount ranging from about 5% to about 25% (w/w) (conveniently between 10-20%) of the total weight of the modified release bead, the weight ratio of the Agent to the binder (when present) ranging from about 4:1 to 19:1 (conveniently between 8:1 to 11:1);
c) a modified release layer that encapsulates the drug layered core bead comprising a modified release polymer, said modified release polymer comprising ethylcellulose or a mixture of ethylcellulose and/or hydroxypropyl cellulose in an amount ranging from about 5% to about 50% (w/w) (conveniently between 20-40%, such as for example 21.91% or 35.79%) of the total weight of the modified release bead, the weight ratio of ethylcellulose to hydroxypropyl cellulose (when present) ranging from about 1:1 to 4:1 (conveniently between 1.3:1 and 3:1); and
d) optionally, a lubricant such as magnesium stearate at about 0.2% of the total weight of the modified release bead.

In one aspect of this embodiment, the Agent is in its free form, i.e. it is present as 2-((3-(4-cyanonaphthalen-1-yl)pyridine-4-yl)thio)-2-methylpropanoic acid.

In one embodiment, typically the Agent (conveniently in its free form, i.e. as 2-((3-(4-cyanonaphthalen-1-yl)pyridine-4-yl)thio)-2-methylpropanoic acid) will be present in the pellet composition of the invention in an amount within the range of from 5 to 50%, suitably from about 10 to 25% and especially from about 10 to 20% by weight of the pellet composition. In a particular group of compositions, the Agent will be present in an amount of about 10-18% by weight of the pellet composition, such as for example 11.5% or 17.2% by weight of the pellet composition.

Conveniently, the modified release pellets range in size from about 150 μm to about 400 μm, more conveniently about 350 μm. As described hereinbefore, the pellet composition will comprise a plurality of pellets that may be packaged, for example, in a capsule such as a gelatin capsule or a capsule formed from a polymer such as HPMCAS, HPMC or starch; dosed as a suspension or slurry in a liquid; dosed in a sachet; or they may be formed into a tablet (e.g. a caplet) or pill by compression or other processes known in the art. Conveniently, the pellet composition comprising the plurality of pellets is packaged in a capsule, such as a gelatin capsule or a capsule formed from a polymer such as HPMCAS, HPMC or starch.

In one embodiment, the modified release pellets of the current invention can be combined with immediate release pellets and packaged together to form a single pharmaceutical composition, for example, in a capsule. Such a composition could be designed to provide a particular release profile comprising both modified release and immediate release components. The immediate release pellets in such an embodiment can comprise an inert core coated with the Agent and optionally additional polymers required to form such a pellet. Conveniently, in one aspect of this embodiment, the amount of immediate release pellets ranges from 5 to 25% of the total weight of the pellets in the composition. More conveniently, the amount of immediate release pellets ranges from 7 to 15% of the total weight of the pellets in the composition.

In one embodiment, the multiparticulates incorporate a delay before the onset of sustained release of the Agent. One embodiment can be illustrated by a multiparticulate comprising a seed core layered with the Agent and coated with a first coating of a polymeric material of the type useful for modified release of the Agent and a second coating of the type useful for delaying release of drugs when the dosage form is ingested. The first coating is applied over and surrounds the Agent layered seed core. The second coating is applied over and surrounds the first coating. The multiparticulate can be prepared by techniques well known in the art. The first coating may be a controlled release coating as known in the art, especially polymer coatings, to fabricate the membrane, as previously discussed. Suitable polymer coating materials, equipment, and coating methods also include those previously discussed.

Suitable materials useful for preparing the second coating on the multiparticulate include polymers known in the art as enteric coatings for delayed-release of pharmaceuticals. These most commonly are pH-sensitive materials such as cellulose acetate phthalate, cellulose acetate trimellitate, hydroxypropyl methyl, cellulose phthalate, poly(vinyl acetate phthalate), and acrylic copolymers such as Eudragit L-100 (Evonik), Eudragit L 30 D-55, Eudragit S 100, Eudragit FS 300, and related materials. The thickness and type of the delayed-release coating is adjusted to give the desired delay property. In general, thicker coatings are more resistant to erosion and, consequently, yield a longer delay as do coatings which are designed to dissolve above pH 7. Preferred coatings typically range from about 10 micron in thickness to about 3 mm in thickness and more preferably 10 um to 500 um. When ingested, the twice-coated multiparticulates pass through the stomach, where the second coating prevents release of the Agent under the acidic conditions prevalent there. When the multiparticulates pass out of the stomach and into the small intestine, where the pH is higher, the second coating erodes or dissolves according to the physicochemical properties of the chosen material. Upon erosion or dissolution of the second coating, the first coating prevents immediate or rapid release of the Agent and modulates the release so as to prevent the production of high concentrations.

In one embodiment, the modified release pharmaceutical composition is a capsule composition comprising a plurality of pellets, wherein the composition comprises: about 5 mg 2-((3-(4-cyanonaphthalen-1-yl)pyridine-4-yl)thio)-2-methylpropanoic acid, about 22 mg microcrystalline seed core spheres, about 0.6 mg HPMC 6 cps, between about 5-8 mg HPC LF (conveniently about 5.6 mg or 6.2 mg), between about 8-10 mg (conveniently about 9.3 mg or 9.9 mg) ethylcellulose and optionally about 0.06 mg magnesium stearate.

In a further embodiment, the modified release pharmaceutical composition is a capsule composition comprising a plurality of pellets, wherein the composition comprises: 10 mg 2-((3-(4-cyanonaphthalen-1-yl)pyridine-4-yl)thio)-2-methylpropanoic acid, about 43-45 mg (conveniently 44.5 mg) microcrystalline seed core spheres, between 0.5-1.5 mg (conveniently 1.1 mg) HPMC 6 cps, between 8-12 mg HPC LF (conveniently 9 mg or 10.1 mg), between about 15-25 mg (conveniently 20.9 mg or 22 mg) ethylcellulose and optionally between 0.1-0.2 mg (conveniently 0.125 mg) magnesium stearate.

In a further embodiment, the modified release pharmaceutical composition is a capsule composition comprising a plurality of pellets, wherein the composition comprises: 4.5 mg 2-((3-(4-cyanonaphthalen-1-yl)pyridine-4-yl)thio)-2-methylpropanoic acid, 14-17 mg (conveniently 15.4 mg) microcrystalline seed core spheres, between 0.5-1.5 mg (conveniently 0.44 mg) HPMC 6 cps, between 1-2 mg HPC LF (conveniently 1.82 mg), between about 1-4.5 mg (conveniently 3.9 mg) ethylcellulose and between 0.01-0.2 mg (conveniently 0.05 mg) magnesium stearate.

In a further embodiment, the modified release pharmaceutical composition is a capsule composition comprising a plurality of pellets, wherein the composition comprises: 6 mg 2-((3-(4-cyanonaphthalen-1-yl)pyridine-4-yl)thio)-2-methylpropanoic acid, 18-22 mg (conveniently 20.8 mg) microcrystalline seed core spheres, between 0.5-1.5 mg (conveniently 0.6 mg) HPMC 6 cps, between 1-3 mg HPC LF (conveniently 2.45 mg), between about 1-6 mg (conveniently 5.25 mg) ethylcellulose and between 0.01-0.2 mg (conveniently 0.07 mg) magnesium stearate.

In a further embodiment, the modified release pharmaceutical composition is a capsule composition comprising a plurality of pellets, wherein the composition comprises: 12 mg 2-((3-(4-cyanonaphthalen-1-yl)pyridine-4-yl)thio)-2-methylpropanoic acid, 40-44 mg (conveniently 41.9 mg) microcrystalline seed core spheres, between 0.5-1.5 mg (conveniently 1.2 mg) HPMC 6 cps, between 1-6 mg HPC LF (conveniently 4.94 mg), between about 1-12 mg (conveniently 10.6 mg) ethylcellulose and between 0.01-0.2 mg (conveniently 0.15 mg) magnesium stearate.

As used herein and unless stated otherwise, it is to be understood that when using the term “bead or “beads” in relation to a multiparticulate formulation, the terms are used synonymously with the terms “pellet” or “pellets”, respectively.

### Medical Uses

The Agent is a potent inhibitor of URAT1 and accordingly the compositions according to the present invention are useful in the treatment of conditions such as those described in International Patent Application WO 2011/159839, which discloses the Agent and also in WO 2013/067425, in which particular methods of using the Agent are disclosed. For example, the composition of the invention is useful for the treatment of disorders of uric acid metabolism including, but are not limited to, polycythemia, myeloid metaplasia, gout, a recurrent gout attack, gouty arthritis, hyperuricaemia, hypertension, a cardiovascular disease, coronary heart disease, Lesch-Nyhan syndrome, Kelley-Seegmiller syndrome, kidney disease, kidney stones, kidney failure, joint inflammation, arthritis, urolithiasis, plumbism, hyperparathyroidism, psoriasis or sarcoidosis. In a particular embodiment, the composition of the invention is useful for the treatment of disorders of uric acid metabolism including, polycythemia, myeloid metaplasia, gout, a recurrent gout attack, gouty arthritis, hyperuricaemia, hypertension, a cardiovascular disease, coronary heart disease, heart failure, Lesch-Nyhan syndrome, Kelley-Seegmiller syndrome, acute or chronic kidney disease, kidney stones, kidney failure, joint inflammation, arthritis, urolithiasis, plumbism, hyperparathyroidism, psoriasis or sarcoidosis.

In a further particular embodiment, the composition of the invention is useful for the treatment of heart failure in patients with elevated serum uric acid levels. In yet a further particular embodiment, the composition of the invention is useful to reduce the risk of cardiovascular death and hospitalization for heart failure patients (NYHA Class II-III) with serum uric acid (sUA) levels of greater than 6 mg/dL.

In a further embodiment, the composition of the invention is useful for the treatment of chronic kidney disease (CKD) in patients with elevated serum uric acid levels. In a particular embodiment, the composition of the invention is useful to reduce the risk of cardiovascular events (MACE) and delay the progression of renal failure (suitably defined as 50% reduction of eGFR or ESRD [dialysis, renal transplantation or SCr >6 mg/dL]) and prevent cardiovascular or renal death in CKD patients (eGFR 25-75 ml/min/1.73 m2) with sUA levels of greater than 6 mg/dL.

A further aspect of the present invention provides a pharmaceutical composition according to the invention as hereinbefore defined for use as a medicament.

The Agent present in the compositions of the invention possesses serum uric acid lowering properties, which are believed to arise from its URAT1 inhibitory activity. Accordingly the composition of the invention is expected to be useful in the treatment of diseases or medical conditions mediated alone or in part by URAT1, i.e. the composition of the invention may be used to produce a URAT1 inhibitory effect in a warm blooded animal in need of such treatment. Thus the composition of the invention provides a method for treating uric acid related disorders characterised by inhibition of URAT1, i.e. the composition of the invention may be used to produce a serum uric acid lowering effect mediated alone or in part by the inhibition of URAT1. Accordingly the compositions of the invention are expected to be useful in the treatment of disorders of uric acid metabolism by providing a serum uric acid lowering effect, particularly in the treatment of URAT1 sensitive disorders such as the disorders hereinbefore described. In a particular embodiment, the composition of the invention provides a method for reducing serum uric acid levels in a human. In yet a further particular embodiment, the composition of the invention provides a method for treating gout. In yet a further particular embodiment, the composition of the invention provides a method for treating hyperuricemia. In yet a further particular embodiment, the composition of the invention provides a method for treating hyperuricemia associated with gout. In yet a further particular embodiment, the composition of the invention provides a method for treating hyperuricemia associated with gout in combination with a xanthine oxidase inhibitor (conveniently allopurinol or febuxostat, more conveniently febuxostat). In yet a further particular embodiment, the composition of the invention provides a method for treating hyperuricemia associated with gout in combination with a xanthine oxidase inhibitor (conveniently allopurinol or febuxostat, more conveniently febuxostat) in patients who warrant additional therapy. In yet a further particular embodiment, the composition of the invention provides a method for the chronic treatment of hyperuricemia in combination with allopurinol or febuxostat when additional therapy is warranted. In yet a further particular embodiment, the composition of the invention provides a method for treating heart failure in patients with elevated serum uric acid levels. In yet a further particular embodiment, the composition of the invention provides a method for reducing the risk of cardiovascular death and hospitalization for heart failure patients (NYHA Class II-III) with serum uric acid (sUA) levels of greater than 6 mg/dL. In yet a further particular embodiment, the composition of the invention provides a method for treating chronic kidney disease (CKD) in patients with elevated serum uric acid levels. In yet a further particular embodiment, the composition of the invention provides a method for reducing the risk of cardiovascular events (MACE) and delaying the progression of renal failure (suitably defined as 50% reduction of eGFR or ESRD [dialysis, renal transplantation or SCr >6 mg/dL]) and preventing cardiovascular or renal death in CKD patients (eGFR 25-75 ml/min/1.73 m2) with sUA levels of greater than 6 mg/dL.

In an embodiment of the invention there is provided, a pharmaceutical composition according to the invention as hereinbefore defined for use in lowering serum uric acid levels in a warm-blooded animal (preferably a human). In another embodiment there is provided a pharmaceutical composition according to the invention as hereinbefore defined for use in the treatment of polycythemia, myeloid metaplasia, gout, a recurrent gout attack, gouty arthritis, hyperuricaemia, hypertension, a cardiovascular disease, coronary heart disease, Lesch-Nyhan syndrome, Kelley-Seegmiller syndrome, kidney disease, kidney stones, kidney failure, joint inflammation, arthritis, urolithiasis, plumbism, hyperparathyroidism, psoriasis or sarcoidosis. In another embodiment there is provided a pharmaceutical composition according to the invention as hereinbefore defined for use in the treatment of polycythemia, myeloid metaplasia, gout, a recurrent gout attack, gouty arthritis, hyperuricaemia, hypertension, a cardiovascular disease, coronary heart disease, heart failure, Lesch-Nyhan syndrome, Kelley-Seegmiller syndrome, acute or chronic kidney disease, kidney stones, kidney failure, joint inflammation, arthritis, urolithiasis, plumbism, hyperparathyroidism, psoriasis or sarcoidosis. In a particular embodiment, there is provided a pharmaceutical composition according to the invention as hereinbefore defined for use in the treatment of gout. In a still further embodiment there is provided a pharmaceutical composition according to the invention for use in the prevention or treatment of uric acid metabolism disorders, which are sensitive to the inhibition of URAT1. In a particular embodiment, there is provided a pharmaceutical composition according to the invention as hereinbefore defined for use in the treatment of gout. In a particular embodiment, there is provided a pharmaceutical composition according to the invention as hereinbefore defined for use in the treatment of heart failure in patients with elevated serum uric acid levels. In a particular embodiment, there is provided a pharmaceutical composition according to the invention as hereinbefore defined for use in reducing the risk of cardiovascular death and hospitalization for heart failure patients (NYHA Class II-III) with serum uric acid (sUA) levels of greater than 6 mg/dL. In yet a further particular embodiment, there is provided a pharmaceutical composition according to the invention as hereinbefore defined for use in the treatment of chronic kidney disease (CKD) in patients with elevated serum uric acid levels. In yet a further particular embodiment, there is provided a pharmaceutical composition according to the invention as hereinbefore defined for use in reducing the risk of cardiovascular events (MACE) and delaying the progression of renal failure (suitably defined as 50% reduction of eGFR or ESRD [dialysis, renal transplantation or SCr >6 mg/dL]) and preventing cardiovascular or renal death in CKD patients (eGFR 25-75 ml/min/1.73 m2) with sUA levels of greater than 6 mg/dL. In yet a further particular embodiment, there is provided a pharmaceutical composition according to the invention as hereinbefore defined for use in the treatment of hyperuricemia. In yet a further particular embodiment, there is provided a pharmaceutical composition according to the invention as hereinbefore defined for use in the treatment of hyperuricemia associated with gout. In yet a further particular embodiment, there is provided a pharmaceutical composition according to the invention as hereinbefore defined for use in the treatment of hyperuricemia associated with gout in combination with a xanthine oxidase inhibitor (conveniently allopurinol or febuxostat, more conveniently febuxostat). In yet a further particular embodiment, there is provided a pharmaceutical composition according to the invention as hereinbefore defined for use in the treatment of hyperuricemia associated with gout in combination with a xanthine oxidase inhibitor (conveniently allopurinol or febuxostat, more conveniently febuxostat) in patients who warrant additional therapy. In yet a further particular embodiment, there is provided a pharmaceutical composition according to the invention as hereinbefore defined for the chronic treatment of hyperuricemia in combination with allopurinol or febuxostat when additional therapy is warranted.

A further aspect of the present invention provides the use of a composition according to the invention as hereinbefore defined in the manufacture of a medicament for use in producing a serum uric acid lowering effect in a warm blooded animal (preferably a human). In another embodiment, there is provided the use of a composition according to the invention as hereinbefore defined in the manufacture of a medicament for use in the treatment of polycythemia, myeloid metaplasia, gout, a recurrent gout attack, gouty arthritis, hyperuricaemia, hypertension, a cardiovascular disease, coronary heart disease, Lesch-Nyhan syndrome, Kelley-Seegmiller syndrome, kidney disease, kidney stones, kidney failure, joint inflammation, arthritis, urolithiasis, plumbism, hyperparathyroidism, psoriasis or sarcoidosis. In another embodiment, there is provided the use of a composition according to the invention as hereinbefore defined in the manufacture of a medicament for use in the treatment of polycythemia, myeloid metaplasia, gout, a recurrent gout attack, gouty arthritis, hyperuricaemia, hypertension, a cardiovascular disease, coronary heart disease, heart failure, Lesch-Nyhan syndrome, Kelley-Seegmiller syndrome, acute or chronic kidney disease, kidney stones, kidney failure, joint inflammation, arthritis, urolithiasis, plumbism, hyperparathyroidism, psoriasis or sarcoidosis. In a particular embodiment, there is provided the use of a composition according to the invention as hereinbefore defined in the manufacture of a medicament for use in the treatment of gout. In a still further embodiment there is provided the use of a composition according to the invention as hereinbefore defined in the manufacture of a medicament for use in the prevention or treatment of uric acid metabolism disorders, which are sensitive to the inhibition of URAT1. In a particular embodiment, there is provided the use of a composition according to the invention as hereinbefore defined in the manufacture of a medicament for use in the treatment of heart failure in patients with elevated serum uric acid levels. In a particular embodiment, there is provided the use of a composition according to the invention as hereinbefore defined in the manufacture of a medicament for use in reducing the risk of cardiovascular death and hospitalization for heart failure patients (NYHA Class II-III) with serum uric acid (sUA) levels of greater than 6 mg/dL. In a particular embodiment, there is provided the use of a composition according to the invention as hereinbefore defined in the manufacture of a medicament for use in the treatment of chronic kidney disease (CKD) in patients with elevated serum uric acid levels. In a particular embodiment, there is provided the use of a composition according to the invention as hereinbefore defined in the manufacture of a medicament for use in reducing the risk of cardiovascular events (MACE) and delaying the progression of renal failure (suitably defined as 50% reduction of eGFR or ESRD [dialysis, renal transplantation or SCr >6 mg/dL]) and preventing cardiovascular or renal death in CKD patients (eGFR 25-75 ml/min/1.73 m2) with sUA levels of greater than 6 mg/dL. In yet a further particular embodiment, there is provided the use of a composition according to the invention as hereinbefore defined in the manufacture of a medicament for use in the treatment of hyperuricemia. In yet a further particular embodiment, there is provided the use of a composition according to the invention as hereinbefore defined in the manufacture of a medicament for use in the treatment of hyperuricemia associated with gout. In yet a further particular embodiment, there is provided the use of a composition according to the invention as hereinbefore defined in the manufacture of a medicament for use in the treatment of hyperuricemia associated with gout in combination with a xanthine oxidase inhibitor (conveniently allopurinol or febuxostat, more conveniently febuxostat). In yet a further particular embodiment, there is provided the use of a composition according to the invention as hereinbefore defined in the manufacture of a medicament for use in the treatment of hyperuricemia associated with gout in combination with a xanthine oxidase inhibitor (conveniently allopurinol or febuxostat, more conveniently febuxostat) in patients who warrant additional therapy. In yet a further particular embodiment, there is provided the use of a composition according to the invention as hereinbefore defined in the manufacture of a medicament for the chronic treatment of hyperuricemia in combination with allopurinol or febuxostat when additional therapy is warranted.

### Combination Therapies

Pharmaceutical compositions of the present invention may be administered alone as a sole therapy or can be administered in addition with one or more other substances and/or treatments. Such conjoint treatment may be achieved by way of the simultaneous, sequential or separate administration of the individual components of the treatment.

For example, therapeutic effectiveness may be enhanced by administration of an adjuvant (i.e., by itself the adjuvant may only have minimal therapeutic benefit, but in combination with another therapeutic agent, the overall therapeutic benefit to the individual is enhanced). Or, by way of example only, the benefit experienced by an individual may be increased by administering the Agent with another therapeutic agent (which also includes a therapeutic regimen) that also has therapeutic benefit. By way of example only, in a treatment for gout, increased therapeutic benefit may result by also providing the individual with another therapeutic agent for gout. Or, the additional therapy or therapies may include, but are not limited to physiotherapy, psychotherapy, radiation therapy, application of compresses to a diseased area, rest, altered diet, and the like.

In the instances where the Agent is administered in combination with other therapeutic agents, the Agent need not be administered via the same route as other therapeutic agents, and may, because of different physical and chemical characteristics, be administered by a different route. For example, the Agent may be administered orally to generate and maintain good blood levels thereof, while the other therapeutic agent may be administered intravenously. The initial administration may be made according to established protocols known in the art, and then, based upon the observed effects, the dosage, modes of administration and times of administration can be modified by the skilled clinician.

The particular choice of other therapeutic agent will depend upon the diagnosis of the attending physicians and their judgment of the condition of the individual and the appropriate treatment protocol. In some embodiments, the additional agent is for the treatment or prophylaxis of gout flares. In some embodiments, the additional agent is a short term treatment for an acute gout attack. In some embodiments, the additional agent is to block the occurrence of flare during the initiation of uric acid lowering therapy. In some embodiments, the additional agent is for the rapid inhibition of the pain and inflammation resulting from the inflammatory response to monosodium UCD. In some embodiments, the additional agent is an inhibitor of cyclooxygenase-1 and -2 enzymes. In some embodiments, the additional agent is a nonsteroidal anti-inflammatory drug (NSAID). Examples of NSAIDs include but are not limited to arylalkanoic acids such as acetaminophen, 2-arylpropionic acids such as ibuprofen, ketorolac and naproxen; n-arylanthranilic acids such as mefenamic acid, meclofenamic acid, oxicams such as piroxicam, meloxicam, arylalkanoic acids such as diclofenac, etodolac, indomethacin, sulindac and COX-2 inhibitors such as celecoxib. In some embodiments, the additional agent is colchicine. In some embodiments, the additional agent is a glucocorticoid receptor (GR) agonist. In some embodiments, the additional agent is a corticosteroid, such as prednisone, prednisolone, triamcinolone and the like. In some embodiments, the additional agent is an IL-1β inhibitor, an IL-1R antagonist, an IL-1β mab, an IL-1R decoy or an anti-IL-1β antibody. In some embodiments, the additional agent is an IL-1 inhibitor. Examples of IL-1 inhibitors include but are not limited to Anakinra, canakinumab, rilonacept and the like. In some embodiments, the additional agent is diacerin (4,5-bis(acetyloxy)-9, 10-dioxo-2-anthracene carboxylic acid. In some embodiments, the additional agent is a phosphodiesterase-4 inhibitor, such as Apremilast. In some embodiments, the additional agent is an anti-05a antibody. In some embodiments, the additional agent is a CXCR2 inhibitor, such as ladarixin (DF-2162). In some embodiments, the additional agent blocks the enzyme responsible for the oxidation of hypoxanthine and xanthine. In some embodiments, the additional agent is a xanthine oxidase inhibitor. Examples of xanthine oxidase inhibitors include but are not limited to Allopurinol (Zyloprim), febuxostat (Uloric, Adenuric), topiroxostat (FYX-051, Topiloric, Uriadec), niraxostat (Y-700) and LC-350189. In some embodiments, the additional agent is an inhibitor of purine nucleoside phosphorylase (PNP), such as ulodesine (BCX4208). In some embodiments, the additional agent is a blocker of purine absorption, such as a Concentrative Nucleoside Transporter Type 2(CNT2). Examples of CNT2 inhibitors include, but are not limited to KGO-2142 and KGO-2173. In some embodiments, the additional agent is a uricase such as Rasburicase or pegloticase. In some embodiments, the additional agent is a uricosuric agent, a urinary alkalinizer or fenofibrate.

In some particular embodiments, the additional agent is a URAT 1 inhibitor, a xanthine oxidase inhibitor, a xanthine dehydrogenase, a xanthine oxidoreductase inhibitor, a purine nucleoside phosphorylase (PNP) inhibitor, a uric acid transporter inhibitor, a glucose transporter (GLUT) inhibitor, a GLUT-9 inhibitor, a solute carrier family 2 (facilitated glucose transporter), member 9 (SLC2A9) inhibitor, an organic anion transporter (OAT) inhibitor, an OAT-4 inhibitor, or combinations thereof.

In some embodiments, the additional agent is selected from 2-((5-bromo-4-(4-cyclopropyl-1-naphthalenyl)-4H-1,2,4-triazol-3-yl)thio)acetic acid, allopurinol, febuxostat (2-(3-cyano-4-isobutoxyphenyl)-4-methyl-1,3-thiazole-5-carboxylic acid), FYX-051 (4-(5-pyridin-4-yl-1H-[1,2,4]triazol-3-yl)pyridine-2-carbonitrile), NIRAXOSTAT (Y-700), LC-350189, probenecid, sulfinpyrazone, benzbromarone, acetaminophen, steroids, nonsteroidal anti-inflammatory drugs (NSAIDs), adrenocorticotropic hormone (ACTH), colchicine, a glucorticoid, an adrogen, a cox-2 inhibitor, a PPAR agonist, naproxen, sevelamer, sibutmaine, troglitazone, proglitazone, another uric acid lowering agent, losartan, fibric acid, benziodarone, salisylate, anlodipine, vitamin C, or combinations thereof. Conveniently, the additional agent is febuxostat.

In a particular embodiment of the invention, compositions of the invention can include at least one additional co-agent in a single dosage form to provide a fixed-combination. In this embodiment, the dosage form could comprise multiparticulates or single unit dosage forms (e.g. tablets) of the current invention containing the Agent along with an additional co-agent formulated as a powder, multiparticulate or single unit dosage (e.g. a tablet). Conveniently, the additional co-agent in this embodiment is febuxostat. Conveniently, in one aspect of this embodiment, the fixed combination comprises a capsule containing a first plurality of pellets containing the Agent formulated in accordance with the current invention and a second plurality of pellets or granules (conveniently granules) containing the additional co-agent in immediate release or modified release form. Conveniently, the additional co-agent in this particular embodiment is xanthinse oxidase inhibitor such as allopurinol and febuxostat, conveniently febuxostat. In a particular embodiment, the fixed combination comprises a capsule containing a first plurality of pellets containing the Agent formulated in accordance with the current invention and a second plurality of pellets or granules (conveniently granules) containing febuxostat in immediate release form. Conveniently, in one aspect of this embodiment, the capsule contains a sufficient quantity of febuxostat containing pellets or granules in immediate release form to provide a dose of 40 mg or 80 mg, conveniently 80 mg.

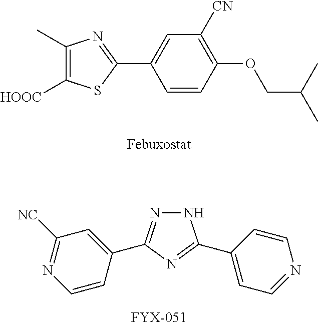

In some further embodiments, the additional agent is for the treatment or prophylaxis of a cardiovascular or metabolic disease. In a particular embodiment, the additional agent is an anti-diabetic agent, for example a sodium-glucose co-transporter 2 inhibitor (SLGT2). In a particular embodiment, the additional agent is selected from dapagliflozin, empagliflozin, canagliflozin and ipragliflozin. In a particular embodiment, the additional agent is dapagliflozin. In a particular embodiment, the pharmaceutical compositions of the present invention may be administered in combination with a xanthine oxidase inhibitor and an SLGT2 inhibitor. In yet a particular embodiment, the pharmaceutical compositions of the present invention is administered in combination with febuxostat and an SLGT2 inhibitor (conveniently dapagliflozin). In one aspect of this embodiment, the compositions of the invention include at least one additional co-agent, such as febuxostat, in a single dosage form to provide a fixed-combination. In this particular embodiment, the dosage form could comprise multiparticulates or single unit dosage forms (e.g. tablets) of the current invention containing the Agent along with an additional co-agent formulated as a powder, multiparticulate or single unit dosage (e.g. a tablet). Conveniently, the fixed dose combination containing the Agent and additional co-agent, such as febuxostat, can also include an SLGT2 inhibitor such as dapagliflozin. Alternatively, the fixed dose combination containing the Agent and additional co-agent, such as febuxostat, can be administered separately but in combination with an SLGT2 inhibitor such as dapagliflozin.

### Kits

In one embodiment, the compositions and methods described herein provide kits for the treatment of disorders, such as the ones described herein. These kits comprise a composition described herein in a container and, optionally, instructions teaching the use of the kit according to the various methods and approaches described herein. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. Kits described herein can be provided, marketed and/or promoted to health providers, including physicians, nurses, pharmacists, formulary officials, and the like. Kits may also, in some embodiments, be marketed directly to the consumer.

The compositions of the invention may be utilized for diagnostics and as research tools. For example, the compositions containing the Agent, either alone or in combination with other compounds, can be used as tools in differential and/or combinatorial analyses to elucidate expression patterns of genes expressed within cells and tissues.

Besides being useful for human treatment, compositions of the invention, may be useful for veterinary treatment of companion animals, exotic animals and farm animals, including mammals, rodents, and the like. Conveniently, such animals include horses, dogs, and cats.

The invention is illustrated below by the following non-limiting examples, wherein unless stated otherwise, the “Agent” is 2-((3-(4-cyanonaphthalen-1-yl)pyridine-4-yl)thio)-2-methylpropanoic acid or a pharmaceutically acceptable salt.

### BRIEF DESCRIPTION OF FIGURES

FIG. 1 shows the mean dissolution profile for the 5 mg immediate release tablet formulation prepared as described in Example 1. The dissolution experiments were carried out in 900 mL SGF (simulated gastric fluid) without pepsin at 37° C. with a paddle speed of 50 rpm (n=6).

FIG. 2 shows the mean dissolution profiles for the MR formulations prepared as described in Example 2-6. The dissolution experiments for MR1, MR2 and MR4 were carried out in 900 mL pH 6.8 50 mM phosphate buffer solution at 37° C. with a paddle speed of 50 rpm (n=6 each). The dissolution experiments for MR3 and MR5 were carried out in a two stage dissolution method, the acid stage was 750 mL of 0.1N HCl and the buffer stage was 1000 mL of pH 6.8 buffer (both stages were at 37° C. with a paddle speed of 50 rpm, n=6).

FIG. 3a shows the mean 2-((3-(4-cyanonaphthalen-1-yl)pyridine-4-yl)thio)-2-methylpropanoic acid plasma concentration-time profile for the 5 mg immediate release formulations dosed under fasted and fed conditions.

FIG. 3b shows the mean 2-((3-(4-cyanonaphthalen-1-yl)pyridine-4-yl)thio)-2-methylpropanoic acid plasma concentration-time profile for immediate release formulations dosed at various levels under fasted conditions.

FIG. 4 shows the mean 2-((3-(4-cyanonaphthalen-1-yl)pyridine-4-yl)thio)-2-methylpropanoic acid plasma concentration-time profile for modified release tablet formulations described in Example 2 through Example 6 at a 5 mg dose in the fasted condition.

FIG. 5 shows the mean 2-((3-(4-cyanonaphthalen-1-yl)pyridine-4-yl)thio)-2-methylpropanoic acid plasma concentration-time profile for modified release tablet formulations described in Example 2 through Example 6 at a 5 mg dose in the fed condition.

FIG. 6 shows the mean 2-((3-(4-cyanonaphthalen-1-yl)pyridine-4-yl)thio)-2-methylpropanoic acid plasma concentration-time profile for the MR4 formulation modified release tablet described in Example 5 and Example 5a dosed as 4×2.5 mg tablets or 1×10 mg tablets in fasted condition and 1×10 mg tablets dosed with low-fat and high-fat meals.

FIG. 7 shows the dissolution profile for the 3-hour pellet formulation prepared as described in Examples 12. The dissolution experiments were carried out in various media with different pH values at 37° C. with a paddle speed of 100 rpm.

FIG. 8 shows the dissolution profile for the 5-hour pellet formulation prepared as described in Examples 13. The dissolution experiments were carried out in various media with different pH values at 37° C. with a paddle speed of 100 rpm.

FIG. 9 shows the dissolution profile for the 8-hour pellet formulation prepared as described in Examples 14. The dissolution experiments were carried out in various media with different pH values at 37° C. with a paddle speed of 100 rpm.

FIG. 10 shows the dissolution profile for the 15-hour pellet formulation prepared as described in Examples 15. The dissolution experiments were carried out in media with pH values of 6.8 or 6.5 at 37° C. with a paddle speed of 100 rpm.

FIG. 11 shows the dissolution profile for the mono-ethanolamine pellet formulation prepared as described in Example 16. The dissolution experiments were carried out in various media with different pH values at 37° C. with a paddle speed of 100 rpm.

FIG. 12 shows the dissolution profile for the mono-ethanolamine pellet formulation prepared in accordance with the process as described in Example 16, with the only exception that the weight amount of PVP and EC was changed from 24% PVP K30 (76% EC) to 23% PVP K30 (77% EC). The dissolution experiments were carried out in various media with different pH values and ionic strength at 37° C. with a paddle speed of 100 rpm.

FIG. 13 shows the mean 2-((3-(4-cyanonaphthalen-1-yl)pyridine-4-yl)thio)-2-methylpropanoic acid plasma concentration-time profile for an oral solution, the MR4 modified release tablet (described in Example 5) and four pellet formulations (as described in Examples 12-17) after administration to Labrador dogs with acidic stomach pH in the fasted state.

FIG. 14 shows the mean 2-((3-(4-cyanonaphthalen-1-yl)pyridine-4-yl)thio)-2-methylpropanoic acid plasma concentration-time profile pellet formulations described in Example 12-15 at a 5 mg 5 hr pellet, 10 mg 8 hr pellet and 10 mg 15 hr pellet dose in the fasted condition.

FIG. 15 shows the mean 2-((3-(4-cyanonaphthalen-1-yl)pyridine-4-yl)thio)-2-methylpropanoic acid plasma concentration-time profile pellet formulations described in Example 12-15 at a 5 mg 5 hr pellet, 10 mg 8 hr pellet and 10 mg 15 hr pellet dose in the fed condition.

FIG. 16 shows the mean 2-((3-(4-cyanonaphthalen-1-yl)pyridine-4-yl)thio)-2-methylpropanoic acid plasma concentration-time profile for the 8-hour pellet formulation described in Example 14 at a 10 mg dose in both the fasted and fed conditions.

FIG. 17 shows the dissolution profile for the pellet formulation prepared as described in Example 22. The dissolution experiment was carried out in pH 6.8 buffer (ionic strength 0.1, 50.0 mM KH2PO4+23.6 mM NaOH) at 37° C. using a paddle speed of 100 rpm.

FIG. 18 shows the dissolution profile for the pellet formulation prepared as described in Example 24. The dissolution experiment was carried out in pH 6.8 buffer (ionic strength 0.1, 50.0 mM KH2PO4+23.6 mM NaOH) at 37° C. using a paddle speed of 100 rpm.

### EXAMPLE 1: PREPARATION OF IMMEDIATE RELEASE TABLET COMPOSITIONS CONTAINING THE AGENT

2-((3-(4-cyanonaphthalen-1-yl)pyridine-4-yl)thio)-2-methylpropanoic acid was prepared in accordance with the methods disclosed in WO 2013/067425 (Example No. 1).

This example formulation was prepared by a conventional direct compression and film coating process. 2-((3-(4-cyanonaphthalen-1-yl)pyridine-4-yl)thio)-2-methylpropanoic acid was micronized using an air jet mill (Fluid Energy Mills). The resultant particle size D₁₀ was less than 1 μm, D₅₀ less than 5 μm and D₉₀ less than 20 μm. Microcrystalline cellulose (Avicel PH-102, FMC International, Philadelphia, Pa., USA), croscarmellose sodium (AcDiSol®, FMC International, Philadelphia, Pa., USA) and colloidal silicon dioxide (CabOSil MSP, Cabot Corporation, Alpharetta, Ga., USA) were all screened prior to use.

The micronized 2-((3-(4-cyanonaphthalen-1-yl)pyridine-4-yl)thio)-2-methylpropanoic acid (23.0 g) and a portion of the microcrystalline cellulose were blended for 5 minutes. The remaining portion of microcrystalline cellulose was added and blended for 5 further minutes (total amount of microcrystalline cellulose is 416.3 g). The croscarmellose sodium (13.8 g) and colloidal silicon dioxide (4.6 g) raw materials were added to the micronized 2-((3-(4-cyanonaphthalen-1-yl)pyridine-4-yl)thio)-2-methylpropanoic acid/microcrystalline cellulose mixture, blended for 5 minutes and then passed through a Comil (Screening Mills) and further blended for an additional 15 minutes. 0.5% (w/w) Magnesium stearate (Hyqual™ 2257, Mallinckrodt Pharmaceuticals, St. Louis, Mo., USA) was screened prior to use and added to the blend and mixed for 5 minutes. The final blend was compressed on a rotary tablet press (Globe Pharma Mini-Press) as 100 mg tablets with a 6.1 mm diameter and approximately 3.5 mm thickness. Tablets were filmed coated in a perforated pan coating system with a hypromellose based aesthetic film coat (15% w/v dispersion of Opadry Blue 03K105000 in purified water) to a target weight gain of 3% w/w. The compositions for the 2-((3-(4-cyanonaphthalen-1-yl)pyridine-4-yl)thio)-2-methylpropanoic acid immediate release tablets, at 5 mg and 20 mg strengths, are presented in Table 1.

TABLE 1
Formulation for 2-((3-(4-cyanonaphthalen-1-yl)pyridine-4-
yl)thio)-2-methylpropanoic acid Immediate Release Tablets

| | | | | |
|---|---|---|---|---|
| | | 5 mg | 20 mg | |
| | | mg/ | mg/ | |
| Ingredient | Grade | tablet | tablet | % w/w |

Tablet Core

| | | | | |
|---|---|---|---|---|
| 2-((3-(4-cyanonaphthalen- | Micronized | 5.0 | 20.0 | 4.83% |
| 1-yl)pyridine-4-yl)thio)-2- | | | | |
| methylpropanoic acid | | | | |
| (micronized) ¹ | | | | |
| Microcrystalline Cellulose² | Avicel PH-102 | 90.5 | 36.0 | 87.44% |
| Croscarmellose Sodium | AcDiSol | 3.0 | 12.0 | 2.90% |
| Colloidal Silicon Dioxide | CabOSil M5P | 1.0 | 4.0 | 0.97% |
| Magnesium Stearate | Hyqual 2257 | 0.5 | 2.0 | 0.48% |

Coating

| | | | | |
|---|---|---|---|---|
| Opadry Blue | 03K105000 | 3.5 | 14.0 | 3.38% |
| Purified Water³ | USP | — | — | — |

| | | | |
|---|---|---|---|
| Total | 103.5 | 414.0 | 100.00% |

¹ Adjusted based on water content and total related substances to provide 5 mg or 20 mg of 2-((3-(4-cyanonaphthalen-1-yl)pyridine-4-yl)thio)-2-methylpropanoic acid per tablet.
²Adjusted after drug substance correction to maintain core tablet weight at 100 mg or 400 mg.
³Processing aid; removed during manufacturing.

### EXAMPLE 2: PREPARATION OF MODIFIED RELEASE HPMC HYDROPHILIC MATRIX TABLET COMPOSITION (MR1)

This example formulation was prepared by a conventional direct compression and film coating process. 2-((3-(4-cyanonaphthalen-1-yl)pyridine-4-yl)thio)-2-methylpropanoic acid was micronized using an air jet mill (Fluid Energy Mills). The resultant particle size D₁₀ was less than 1 μm, D₅₀ less than 5 μm and D₉₀ less than 20 μm.

With the exception of 2-((3-(4-cyanonaphthalen-1-yl)pyridine-4-yl)thio)-2-methylpropanoic acid all raw materials were screen prior to use.

Micronized 2-((3-(4-cyanonaphthalen-1-yl)pyridine-4-yl)thio)-2-methylpropanoic acid (12.50 g), and a portion of the microcrystalline cellulose were blended for 5 minutes. The remaining portion of microcrystalline cellulose was added and blended for 5 further minutes (total amount of microcrystalline cellulose is 381.75 g). The hypromellose (100.0 g, Methocel K100 Premium LV CR, Dow Chemical Company, Midland, Mich., USA) and colloidal silicon dioxide (2.0 g) were added to the micronized 2-((3-(4-cyanonaphthalen-1-yl)pyridine-4-yl)thio)-2-methylpropanoic acid/microcrystalline cellulose blend, mixed for 5 minutes, passed through a Comil (Screening Mills) and further blended for an additional 15 minutes. Magnesium stearate 0.75% (w/w) is added to the blend and mixed for 5 minutes. The final blend was compressed on a rotary tablet press (Manesty Beta Press) as 100 mg tablets with a 6.1 mm diameter and approximately 3.5 mm thickness. The resultant tablet cores were filmed coated in a perforated pan coating system with a hypromellose based aesthetic film coat (15% w/v dispersion of Opadry Blue 03K105000 in purified water) to a target weight gain of 3% w/w. The composition of the MR1 formulation is presented in Table 2.

TABLE 2
Composition of 2-((3-(4-cyanonaphthalen-1-yl)pyridine-
4-yl)thio)-2-methylpropanoic acid modified release
HPMC hydrophilic matrix tablet (MR1)

| | | |
|---|---|---|
| Ingredients | % w/w | mg/tablet |

Tablet Core

| | | |
|---|---|---|
| 2-((3-(4-cyanonaphthalen-1-yl)pyridine- | 2.43 | 2.50 |
| 4-yl)thio)-2-methylpropanoic acid ¹ | | |
| (Micronized) | | |
| Hypromellose | 19.42 | 20.00 |
| (Methocel K100 Premium LVCR) | | |
| Microcrystalline Cellulose² | 74.13 | 76.35 |
| (Avicel PH-102) | | |
| Colloidal Silicon Dioxide | 0.39 | 0.40 |
| (CabOSil M5P) | | |
| Magnesium Stearate | 0.73 | 0.75 |
| (Hyqual 2257) | | |

Film Coat

| | | |
|---|---|---|
| Opadry Blue (03K105000) | 2.91 | 3.00 |
| Purified Water³ (USP) | — | — |
| Total Tablet Weight | 100.00 | 103.0 |

¹ Adjusted based upon water content and total related substances to provide 2.5 mg per tablet
²Quantity of microcrystalline cellulose to be adjusted after drug substance correction to maintain target core tablet weight at 100 mg
³Purified water is removed during processing.

### EXAMPLE 3: PREPARATION OF MODIFIED RELEASE HPMC/POLYETHYLENE OXIDE HYDROPHILIC MATRIX TABLET COMPOSITION (MR2)

This example formulation was prepared by a conventional direct compression and film coating process. 2-((3-(4-cyanonaphthalen-1-yl)pyridine-4-yl)thio)-2-methylpropanoic acid was micronized using an air jet mill (Fluid Energy Mills). The resultant particle size D₁₀ was less than 1 μm, D₅₀ less than 5 μm and D₉₀ less than 20 μm.

With the exception of 2-((3-(4-cyanonaphthalen-1-yl)pyridine-4-yl)thio)-2-methylpropanoic acid all raw materials were screen prior to use.

Micronized 2-((3-(4-cyanonaphthalen-1-yl)pyridine-4-yl)thio)-2-methylpropanoic acid (12.50 g) and a portion of microcrystalline cellulose were blended for 5 minutes. The remaining portion of microcrystalline cellulose was added and blended for 5 further minutes (total amount of microcrystalline cellulose used was 381.75 g). The lactose monohydrate (40.0 g, Foremost Farms, Rothschild, Wis., USA), hypromellose (75.0 g, Methocel K100 Premium LV CR, Dow Chemicals), Polyethylene Oxide (50.0 g, PolyOx WSR N750, Dow Chemicals) and colloidal silicon dioxide (1.5 g) were added to the micronized 2-((3-(4-cyanonaphthalen-1-yl)pyridine-4-yl)thio)-2-methylpropanoic acid/microcrystalline cellulose blend, blended for 5 minutes, passed through a Comil (Screening Mills) and further blended for an additional 15 minutes. Magnesium stearate 0.5% (w/w) was added to the blend and mixed. The final blend was compressed on a rotary tablet press (Manesty Beta Press) as 100 mg tablets with a 6.1 mm diameter and approximately 3.5 mm thickness. The resultant tablet cores were filmed coated in a perforated pan coating system with a hypromellose based aesthetic film coat (15% w/v dispersion of Opadry Blue 03K105000 in purified water) to a target weight gain of 3% w/w. The composition of the MR2 formulation is presented in Table 3.

TABLE 3
Composition of 2-((3-(4-cyanonaphthalen-1-yl)pyridine-4-yl)thio)-2-
methylpropanoic acid modified release HPMC/Polyethylene Oxide
hydrophilic matrix tablet (MR2)

| | | |
|---|---|---|
| Ingredients | % w/w | mg/tablet |

Tablet Core

| | | |
|---|---|---|
| 2-((3-(4-cyanonaphthalen-1-yl)pyridine-4-yl)thio)- | 2.43 | 2.50 |
| 2-methylpropanoic acid¹ (Micronized) | | |
| Hypromellose | 14.56 | 15.00 |
| (Methocel K100LV CR) | | |
| Polyethylene Oxide | 9.71 | 10.00 |
| (PolyOx WSR N750) | | |
| Microcrystalline Cellulose² | 61.84 | 63.70 |
| (Avicel PH-102) | | |
| Lactose Monohydrate | 7.77 | 8.00 |
| (FastFlo 316) | | |
| Colloidal Silicon Dioxide | 0.29 | 0.30 |
| (CabOSil M5P) | | |
| Magnesium Stearate | 0.49 | 0.50 |
| (Hyqual 2257) | | |

Film Coat

| | | |
|---|---|---|
| Opadry Blue (03K105000) | 2.91 | 3.00 |
| Purified Water³ (USP) | — | — |
| Total Tablet Weight | 100.00 | 103.0 |

¹Adjusted based upon water content and total related substances to provide 2.5 mg per tablet
²Quantity of microcrystalline cellulose to be adjusted after drug substance correction to maintain target core tablet weight at 100 mg
³Purified water is removed during processing.

### EXAMPLE 4: PREPARATION OF DELAYED RELEASE TABLET COMPOSITION (MR3)

This example formulation was prepared by a conventional direct compression and film coating process. 2-((3-(4-cyanonaphthalen-1-yl)pyridine-4-yl)thio)-2-methylpropanoic acid was micronized using an air jet mill (Fluid Energy Mills). The resultant particle size D₁₀ was less than 1 μm, D₅₀ less than 5 μm and D₉₀ less than 20 μm.

With the exception of 2-((3-(4-cyanonaphthalen-1-yl)pyridine-4-yl)thio)-2-methylpropanoic acid all raw materials were screen prior to use.

The micronized 2-((3-(4-cyanonaphthalen-1-yl)pyridine-4-yl)thio)-2-methylpropanoic acid (50.0 g) and a portion of the microcrystalline cellulose were blended for 5 minutes. The remaining portion of microcrystalline cellulose was added and blended for 5 further minutes (total amount of microcrystalline cellulose used was 1860.0 g). The croscarmellose sodium (60.0 g) and colloidal silicon dioxide (20.0 g) raw materials were added to the micronized 2-((3-(4-cyanonaphthalen-1-yl)pyridine-4-yl)thio)-2-methylpropanoic acid/microcrystalline cellulose mixture, blended for 5 minutes, passed through a Comil (Screening Mills) and further blended for an additional 15 minutes. Magnesium stearate 0.5% (w/w) was screened prior to use and added to the blend and mixed for 5 minutes. The final blend was compressed on a rotary tablet press (Manesty Beta Press) as 100 mg tablets with a 6.1 mm diameter and approximately 3.5 mm thickness.

The above tablets were film coated with an enteric polymer coating. The enteric polymer coating was comprised of hypromellose acetate succinate (Aqoat AS-HF, Shin-Etsu Chemical Company, Ltd., Tokyo, Japan) 29.8 g, triethyl citrate (Vertellus Performance Materials, Inc. Indianapolis, Ind., USA) 10.4 g, talc (Brenntag Specialties, Inc., Luzenac, Val Chisone, Italy) 9.0 g, and sodium lauryl sulfate (Spectrum Chemical Manufacturing Company, Gardena, Calif.) 0.9 g. The coating was applied to the tablets using a perforated pan coater to an approximately 10% weight gain. Tablets were subsequently film coated in a perforated pan coating system with a hypromellose based aesthetic film coat (15% w/v dispersion of Opadry Blue 03K105000 in purified water) to a target weight gain of 3% w/w. The compositions for the 2-((3-(4-cyanonaphthalen-1-yl)pyridine-4-yl)thio)-2-methylpropanoic acid MR3 formulations are presented in Table 4.

TABLE 4
Composition of 2-((3-(4-cyanonaphthalen-1-yl)pyridine-4-yl)thio)-2-
methylpropanoic acid delayed release tablet (MR3)

| | | |
|---|---|---|
| Ingredients | % w/w | mg/tablet |

Tablet Core

| | | |
|---|---|---|
| 2-((3-(4-cyanonaphthalen-1-yl)pyridine-4-yl)thio)- | 2.21 | 2.50 |
| 2-methylpropanoic acid ¹ (Micronized) | | |
| Microcrystalline Cellulose² | 82.08 | 93.00 |
| (Avicel PH-102) | | |
| Croscarmellose Sodium | 2.65 | 3.00 |
| (Ac-Di-Sol) | | |
| Colloidal Silicon Dioxide | 0.88 | 1.00 |
| (CabOSil M5P) | | |
| Magnesium Stearate | 0.44 | 0.50 |
| (Hyqual 2257) | | |

Enteric Coat

| | | |
|---|---|---|
| Hypromellose Acetate Succinate | 5.25 | 5.95 |
| (Aqoat AS-HF) | | |
| Triethyl Citrate (USP/EP) | 1.84 | 2.08 |
| Talc (Pharma M) | 1.58 | 1.79 |
| Sodium Lauryl Sulfate (USP/EP) | 0.16 | 0.18 |
| Purified Water³ (USP) | — | — |

Aesthetic Coat

| | | |
|---|---|---|
| Opadry Blue (03K105000) | 2.91 | 3.30 |
| Purified Water³ (USP) | — | — |
| Total Tablet Weight | 100.00 | 113.3 |

¹ Adjusted based upon water content and total related substances to provide 2.5 mg per tablet
²Quantity of microcrystalline cellulose to be adjusted after drug substance correction to maintain target core tablet weight at 100 mg
³Purified water is removed during processing.

### EXAMPLE 5: PREPARATION OF MODIFIED RELEASE HPMC HYDROPHILIC MATRIX TABLET COMPOSITION (MR4)

This example formulation was prepared by a conventional direct compression and film coating process. 2-((3-(4-cyanonaphthalen-1-yl)pyridine-4-yl)thio)-2-methylpropanoic acid was micronized using an air jet mill (Fluid Energy Mills). The resultant particle size D₁₀ was less than 1 μm, D₅₀ less than 5 μm and D₉₀ less than 20 μm.

With the exception of 2-((3-(4-cyanonaphthalen-1-yl)pyridine-4-yl)thio)-2-methylpropanoic acid all raw materials were screen prior to use.

Micronized 2-((3-(4-cyanonaphthalen-1-yl)pyridine-4-yl)thio)-2-methylpropanoic acid (625.0 g) and a portion of microcrystalline cellulose were blended for 8 minutes. The second portion of microcrystalline cellulose was added and blended for 8 minutes. The hypromellose (7500.0 g, Methocel K100M Premium DC), colloidal silicon dioxide (125 g) and a third portion of microcrystalline cellulose (total amount of microcrystalline cellulose used was 16687.5 g) were added to the micronized 2-((3-(4-cyanonaphthalen-1-yl)pyridine-4-yl)thio)-2-methylpropanoic acid/microcrystalline cellulose mixture, blended for 5 minutes and then passed through a Comil (Screening Mills) for further blending for an additional 24.4 minutes. Magnesium stearate 0.25% (w/w) was screened prior to use and added to the blend and mixed for 8 minutes. The final blend was compressed on a rotary tablet press (Manesty Beta Press) as 100 mg tablets with a 6.1 mm diameter and approximately 3.5 mm thickness. The resultant tablet cores were filmed coated in a perforated pan coating system with a hypromellose based aesthetic film coat (15% w/v dispersion of Opadry Blue 03K105000 in purified water) to a target weight gain of 3% w/w. The composition of the MR4 formulation is presented in Table 5.

TABLE 5
Composition of 2-((3-(4-cyanonaphthalen-1-yl)pyridine-4-
yl)thio)-2-methylpropanoic acid modified release HPMC
K100M hydrophilic matrix tablet (MR4)

| | | |
|---|---|---|
| Ingredients | % w/w | mg/tablet |

Tablet Core

| | | |
|---|---|---|
| 2-((3-(4-cyanonaphthalen-1-yl)pyridine-4- | 2.43 | 2.50 |
| yl)thio)-2-methylpropanoic acid ¹ (Micronized) | | |
| Hypromellose | 29.13 | 30.00 |
| (Methocel K100M Premium DC) | | |
| Microcrystalline Cellulose² | 64.81 | 66.75 |
| (Avicel PH-102) | | |
| Colloidal Silicon Dioxide | 0.49 | 0.50 |
| (CabOSil M5P) | | |
| Magnesium Stearate | 0.24 | 0.25 |
| (Hyqual 2257) | | |

Film Coat

| | | |
|---|---|---|
| Opadry Blue (03K105000) | 2.91 | 3.00 |
| Purified Water³ (USP) | — | — |
| Total Tablet Weight | 100.00 | 103.0 |

¹ Adjusted based upon water content and total related substances to provide 2.5 mg per tablet
²Quantity of microcrystalline cellulose to be adjusted after drug substance correction to maintain target core tablet weight at 100 mg
³Purified water is removed during processing.

### EXAMPLE 5A: PREPARATION OF MODIFIED RELEASE HPMC HYDROPHILIC MATRIX TABLET COMPOSITION, 10 MG DOSE (MR4)

This example formulation was prepared by a conventional direct compression and film coating process. 2-((3-(4-cyanonaphthalen-1-yl)pyridine-4-yl)thio)-2-methylpropanoic acid was micronized using an air jet mill (Fluid Energy Mills). The resultant particle size D₁₀ was less than 1 μm, D₅₀ less than 5 μm and D₉₀ less than 20 μm.

With the exception of 2-((3-(4-cyanonaphthalen-1-yl)pyridine-4-yl)thio)-2-methylpropanoic acid all raw materials were screened prior to use.

Micronized 2-((3-(4-cyanonaphthalen-1-yl)pyridine-4-yl)thio)-2-methylpropanoic acid (25.0 g) and the microcrystalline cellulose (958.1 g) were blended for 5 minutes. Lactose monohydrate (506.0 g) hypromellose (396.0 g, Benecel K100M PHARM, Ashland) and colloidal silicon dioxide (5.0 g) were added to the micronized 2-((3-(4-cyanonaphthalen-1-yl)pyridine-4-yl)thio)-2-methylpropanoic acid/microcrystalline cellulose mixture, blended for 5 minutes then passed through a Comil (Screening Mills) and further blended for an additional 15 minutes. Magnesium stearate 0.25% (w/w) was screened prior to use and added to the blend and mixed for 5 minutes. The final blend was compressed on a rotary tablet press (Manesty Beta Press) as a 5×9.5 mm 180 mg tablets and approximately 4.1 mm thickness. The resultant tablet cores were filmed coated in a perforated pan coating system with a hypromellose based aesthetic film coat (15% w/v dispersion of Opadry White 03K18416 in purified water) to a target weight gain of 3% w/w. The composition of the 10 mg MR4 10 mg tablet formulation is presented in Table 6.

TABLE 6
Composition of 10 mg 2-((3-(4-cyanonaphthalen-1-yl)pyridine-
4-yl)thio)-2-methylpropanoic acid modified release
HPMC K100M hydrophilic matrix tablet (MR4)

| Ingredients | % w/w | mg/tablet |
|---|---|---|
| 2-((3-(4-cyanonaphthalen-1-yl)pyridine-4- | 5.39 | 10.00 |
| yl)thio)-2-methylpropanoic acid² (Micronized) | | |
| Hypromellose | 19.42 | 36.00 |
| (Methocel K100M Premium DC) | | |
| Microcrystalline Cellulose | 46.98 | 87.10 |
| (Avicel PH-102) | | |
| Lactose Monohydrate | 24.81 | 46.00 |
| (Foremost FastFlo 316) | | |
| Colloidal Silicon Dioxide | 0.24 | 0.45 |
| (CabOSil M5P) | | |
| Magnesium Stearate | 0.24 | 0.45 |
| (Hyqual 2257) | | |
| Opadry White (03K18416) | 2.91 | 5.40 |
| Purified Water² (USP) | — | — |
| Total Tablet Weight | 100.0 | 185.40 |

¹Adjusted based upon water content and total related substances to provide 10 mg per tablet
²Purified water is removed during processing

### EXAMPLE 6: PREPARATION OF DELAYED RELEASE HPMC HYDROPHILIC MATRIX TABLET COMPOSITION (MR5)

This example formulation was prepared by a conventional direct compression and film coating process. 2-((3-(4-cyanonaphthalen-1-yl)pyridine-4-yl)thio)-2-methylpropanoic acid was micronized using an air jet mill (Fluid Energy Mills). The resultant particle size D₁₀ was less than 1 μm, D₅₀ less than 5 μm and D₉₀ less than 20 μm.

With the exception of 2-((3-(4-cyanonaphthalen-1-yl)pyridine-4-yl)thio)-2-methylpropanoic acid all raw materials were screen prior to use.

Micronized 2-((3-(4-cyanonaphthalen-1-yl)pyridine-4-yl)thio)-2-methylpropanoic acid (25.0 g) and a portion of the microcrystalline cellulose were blended for 5 minutes. The remaining portion of microcrystalline cellulose was added (total amount used 672.5 g) and blended for 5 minutes. The hypromellose (300.0 g, Methocel K100M Premium DC) was added to the micronized 2-((3-(4-cyanonaphthalen-1-yl)pyridine-4-yl)thio)-2-methylpropanoic acid/microcrystalline cellulose mixture, blended for 5 minutes and then passed through a Comil (Screening Mills) and further blended for an additional 15 minutes. Magnesium stearate 0.25% (w/w) was screened prior to use and added to the blend and mixed for 8 minutes. The final blend was compressed on a rotary tablet press (Globe Pharma Mini Press) as 100 mg tablets with a 6.1 mm diameter and approximately 3.5 mm thickness.

The above tablets were film coated with an enteric polymer coating. The enteric polymer coating was comprised of Methacrylic Acid Copolymer Dispersion (Eudragit L30D-55, Evonik Industries AG, Germany) 43.1 g, Triethyl citrate 1.3 g, and Talc 2.5 g. The coating was applied to tablets using a perforated pan coater to an approximately 5% weight gain. The resultant tablets were then filmed coated in a perforated pan coating system with a hypromellose based aesthetic film coat (15% w/v dispersion of Opadry Blue 03K105000 in purified water) to a target weight gain of 3% w/w. The composition of the MR5 formulation is presented in Table 7.

TABLE 7
Composition of 2-((3-(4-cyanonaphthalen-1-yl)pyridine-4-yl)thio)-
2-methylpropanoic acid delayed release HPMC
K100M hydrophilic matrix tablet (MR5)

| | | |
|---|---|---|
| Ingredients | % w/w | mg/tablet |

Tablet Core

| | | |
|---|---|---|
| 2-((3-(4-cyanonaphthalen-1-yl)pyridine-4- | 2.31 | 2.50 |
| yl)thio)-2-methylpropanoic acid ¹ (Micronized) | | |
| Hypromellose | 27.74 | 30.00 |
| (Methocel K100M Premium DC) | | |
| Microcrystalline Cellulose² | 62.18 | 67.25 |
| (Avicel PH-102) | | |
| Magnesium Stearate | 0.23 | 0.25 |
| (Hyqual 2257) | | |

Enteric Coat

| | | |
|---|---|---|
| Methacrylic Acid Copolymer | 3.56 | 3.85 |
| (Eudragit L30D-55) | | |
| Triethyl Citrate (USP/EP) | 0.37 | 0.40 |
| Talc (Pharma M) | 0.69 | 0.75 |
| Purified Water³ (USP) | — | — |

Aesthetic Coat

| | | |
|---|---|---|
| Opadry Blue (03K105000) | 2.91 | 3.15 |
| Purified Water³ (USP) | N/A | N/A |
| Total Tablet Weight | 100.00 | 108.15 |

¹Adjusted based upon water content and total related substances to provide 2.5 mg per tablet
²Quantity of microcrystalline cellulose to be adjusted after drug substance correction to maintain target core tablet weight at 100 mg
³Purified water is removed during processing.

### EXAMPLE 7: DISSOLUTION TESTING OF IMMEDIATE RELEASE AND MODIFIED RELEASE TABLET FORMULATIONS

### Methods

Dissolution of immediate release tablets were performed according to the general procedure of the United States Pharmacopeia Apparatus II (paddle) for immediate release dosage forms. Aliquots of the dissolution test media were collected and filtered at specific time intervals and analyzed by reverse phase HPLC using isocratic elution and UV detection at 226 nm. The HPLC method conditions were: Analytical Column: Reverse phase HPLC C₁₈ column, YMC ODS-AQ, 4.6×150 mm, 120 Å, 3 μm (Part # AQ12S031546WT); Eluent: 60% 10 mM KH₂PO₄, pH 2.4/40% Acetonitrile; 20 or 50 μL injection volume (depending on dosage strength), 1.0 mL/min flow rate, 35° C. column temperature; ambient sample temperature; 8 minute run time. The release of 2-((3-(4-cyanonaphthalen-1-yl)pyridine-4-yl)thio)-2-methylpropanoic acid was determined by comparing the peak responses of the sample chromatograms to the peak responses of the standard chromatograms. 900 mL SGF (simulated gastric fluid) without pepsin at 37° C. and a paddle speed of 50 rpm is used. The SGF was prepared by adding 12.0 g of sodium chloride to 42.0 mL of concentrated hydrochloric acid brought to 6 L with deionized water. The solution had a pH of about 1.2.

Dissolution of MR1, MR2 and MR4 tablets were performed according to the general procedure of the United States Pharmacopeia Apparatus II (paddle) for extended release dosage forms. Aliquots of the dissolution test media are collected and filtered at specific time intervals and analyzed by reverse phase HPLC using isocratic elution and UV detection at 226 nm. The HPLC method conditions were: Analytical Column: YMC ODS-AQ, 4.6×150 mm, 120 Å, 3 μm (Part # AQ12S031546WT); Eluent: 60% 10 mM KH₂PO₄, pH 2.4/40% Acetonitrile; 20 or 50 μL injection volume (depending on dosage strength), 1.0 mL/min flow rate, 35° C. column temperature; ambient sample temperature; 8 minute run time. The release of 2-((3-(4-cyanonaphthalen-1-yl)pyridine-4-yl)thio)-2-methylpropanoic acid is determined by comparing the peak responses of the sample chromatograms to the peak responses of the standard chromatograms obtained concomitantly. The method uses Spiral Stainless Steel Capsule Sinkers to hold the tablets or capsules. 900 mL pH 6.8 50 mM phosphate buffer solution was used at 37° C. and a paddle speed of 50 rpm is used. The buffer was prepared by adding 122.4 g of KH₂PO₄ dissolved in approximately 16 L of deionized water, pH adjusted to 6.8±0.1 with 1 N sodium hydroxide, then brought to a total of 18 L with deionized water.

Dissolution of MR3 tablets was performed according to the general procedure of the United States Pharmacopeia Apparatus II (paddle) for delayed release dosage forms using a two stage dissolution method. Aliquots of the dissolution test media were collected and filtered at specific time intervals and analyzed by reverse phase HPLC using isocratic elution and UV detection at 226 nm. The HPLC method conditions were: Analytical Column: YMC ODS-AQ, 4.6×150 mm, 120 Å, 3 μm (Part # AQ12S031546WT); Eluent: 60% 10 mM KH₂PO₄, pH 2.4/40% Acetonitrile; 20 or 50 μL injection volume (depending on dosage strength), 1.0 mL/min flow rate, 35° C. column temperature; ambient sample temperature; 8 minute run time. The release of 2-((3-(4-cyanonaphthalen-1-yl)pyridine-4-yl)thio)-2-methylpropanoic acid was determined by comparing the peak responses of the sample chromatograms to the peak responses of the standard chromatograms obtained concomitantly. The acid stage is 750 mL of 0.1N HCl and the buffer stage is 1000 mL of pH 6.8 buffer. Both stages are at 37° C. and use a paddle speed of 50 rpm. The acid stage is from the first 0 to 2 hours. At 2 hours, the pH is increased to 6.8 by addition of 250 mL of 0.20 M Na3PO4 buffer solution to the media. The buffer media was prepared by adding 152 g of Na₃PO₄.12H₂O dissolved in 2 L of deionized water, pH adjusted as necessary with 2 N NaOH or 2 N HCl such that the final buffer solution (after addition to the 750 mL of acid stage media) is pH is 6.8±0.1.

Dissolution of MR5 tablets was performed according to the general procedure of the United States Pharmacopeia Apparatus II (paddle) for delayed release dosage forms using a two stage dissolution method. Aliquots of the dissolution test solutions were collected and filtered at specific time intervals and analyzed by reverse phase HPLC using isocratic elution and UV detection at 226 nm. The HPLC method conditions were: Analytical Column: YMC ODS-AQ, 4.6×150 mm, 120 Å, 3 μm (Part # AQ12S031546WT); Eluent: 60% 10 mM KH₂PO₄, pH 2.4/40% Acetonitrile; 20 or 50 μL injection volume (depending on dosage strength), 1.0 mL/min flow rate, 35° C. column temperature; ambient sample temperature; 8 minute run time. The release of 2-((3-(4-cyanonaphthalen-1-yl)pyridine-4-yl)thio)-2-methylpropanoic acid was determined by comparing the peak responses of the sample chromatograms to the peak responses of the standard chromatograms. The acid stage was 500 or 900 mL of 0.1N HCl (depending on dosage strength) and the buffer stage was 500 or 900 mL (depending on dosage strength) of pH 6.8 50 mM phosphate buffer solution (122.4 g of KH₂PO₄ dissolved in approximately 16 L of deionized water, pH adjusted to 6.8±0.1 with 1 N sodium hydroxide, then brought to a total of 18 L with deionized water). Both stages are at 37° C. and use a paddle speed of 50 rpm. The acid stage is from the first 0 to 2 hours followed by then the same dosage unit being transferred into the buffer stage medium. This might be accomplished by removing from the apparatus the vessel containing the acid and replacing it with another vessel containing the buffer and transferring the dosage unit to the vessel containing the buffer. Continue to operate the apparatus. As an alternative, a different dissolution apparatus prepared according to the conditions specified above could be used for the Buffer stage.

### Dissolution Results

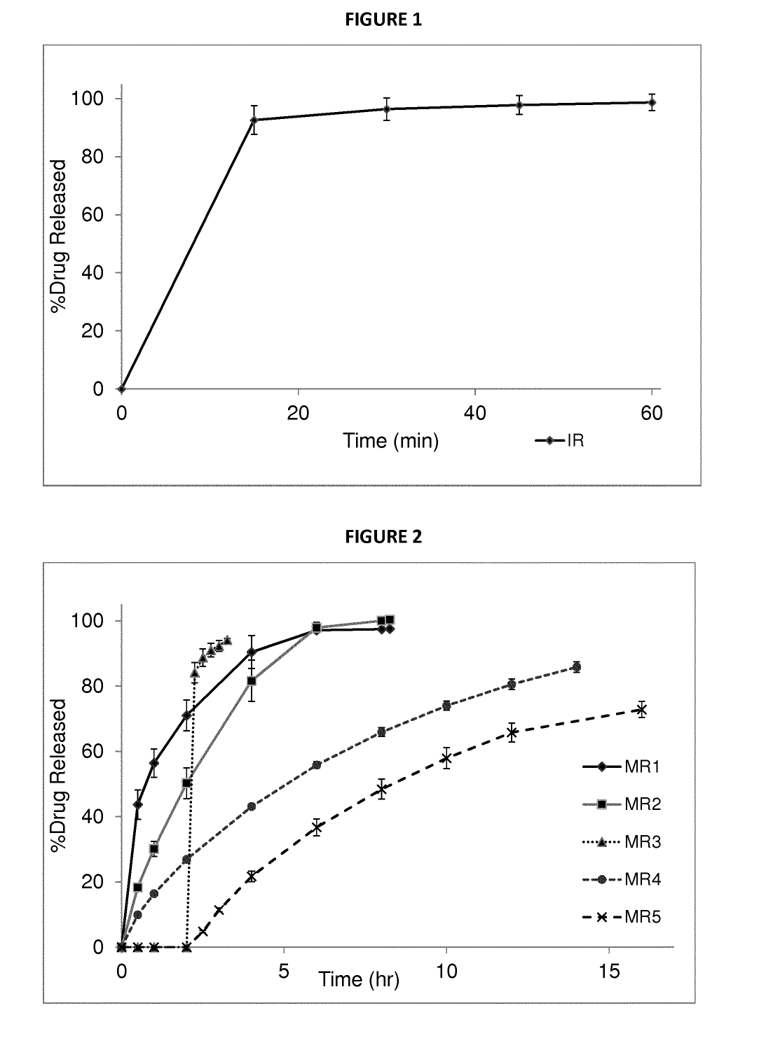

FIG. 1 shows the dissolution profile for the 5 mg immediate release formulation described in Example 1 (n=6). FIG. 2 shows the dissolution profile for the MR formulations described in Examples 2-6 (n=6).

The dissolution data for the immediate release tablets show that dissolution is rapid and >80% of 2-((3-(4-cyanonaphthalen-1-yl)pyridine-4-yl)thio)-2-methylpropanoic acid is released within 15 minutes. Modified release matrix tablet formulation MR1 and MR2 show 80% of 2-((3-(4-cyanonaphthalen-1-yl)pyridine-4-yl)thio)-2-methylpropanoic acid released in 3 hours and 4 hours respectively. The MR3 formulation showed no release over 2 hours at pH 1.1 with immediate release following the media pH change to 6.8. The MR4 formulation showed 80% of 2-((3-(4-cyanonaphthalen-1-yl)pyridine-4-yl)thio)-2-methylpropanoic acid released in 12 hours. The MR5 formulation shows no 2-((3-(4-cyanonaphthalen-1-yl)pyridine-4-yl)thio)-2-methylpropanoic acid release for 2 hours at pH 1.1. 2-((3-(4-cyanonaphthalen-1-yl)pyridine-4-yl)thio)-2-methylpropanoic acid dissolution profile from the MR5 formulation is similar to that seen for MR4 following the dissolution media pH change to pH 6.8.

### EXAMPLE 8: SINGLE DOSE PHASE I CLINICAL TRIAL—IMMEDIATE RELEASE FORMULATIONS

A phase 1, randomized, double-blind, placebo-controlled study in healthy adult male volunteers evaluated single rising doses and the preliminary food effect for 2-((3-(4-cyanonaphthalen-1-yl)pyridine-4-yl)thio)-2-methylpropanoic acid at 6 dose groups with 8 subjects per group. Under fed conditions, the subjects were required to fast overnight for at least 10 hours before dosing, then receive study medication 30 minutes after completing a standard moderate fat breakfast that did not include high fructose corn syrup. Subjects in each dose group were randomized to receive a single dose of either 2-((3-(4-cyanonaphthalen-1-yl)pyridine-4-yl)thio)-2-methylpropanoic acid (6 subjects) or placebo (2 subjects). 2-((3-(4-cyanonaphthalen-1-yl)pyridine-4-yl)thio)-2-methylpropanoic acid was supplied as 1 mg oral solution, 2 mg oral solution, 5 mg tablets, or 20 mg tablets (depending on dose level).

Segment A evaluated single rising doses of 2 mg, 5 mg, 20 mg and 40 mg and the preliminary food effect of 2-((3-(4-cyanonaphthalen-1-yl)pyridine-4-yl)thio)-2-methylpropanoic acid at the 5 mg and 20 mg doses followed by multiple ascending doses of 1 mg, 5 mg and 10 mg qd in Segment B.

| Segment | Group | Dose (mg) | Dietary State | Dose Form |
|---|---|---|---|---|
| A | 1 | 20 mg | Fasted | 1 × 20 mg tablet |
| | 2 | 40 mg | Fasted | 2 × 20 mg tablet |
| | 3 | 20 mg | Fed | 1 × 20 mg tablet |
| | 4 | 5 mg | Fasted | 1 × 5 mg tablet |
| | 5 | 2 mg | Fasted | oral solution |
| | 6 | 5 mg | Fed | 1 × 5 mg tablet |
| B | 1 | 5 mg qd | Fasted | 1 × 5 mg tablet |
| | 2 | 10 mg qd | Fasted | 2 × 5 mg tablet |
| | 3 | 1 mg qd | Fasted | oral solution |

The oral solution was prepared by the clinical pharmacist in bulk within 24 hours of administration. The oral solution of 2-((3-(4-cyanonaphthalen-1-yl)pyridine-4-yl)thio)-2-methylpropanoic acid (0.033 mg/mL concentration) was prepared as a mixture of the appropriate amount of 2-((3-(4-cyanonaphthalen-1-yl)pyridine-4-yl)thio)-2-methylpropanoic acid powder, anhydrous dibasic sodium phosphate, and sterile water for irrigation; placebo oral solution was prepared as a vehicle using anhydrous dibasic sodium phosphate and sterile water for irrigation. The immediate release tablets were prepared as described in Example 1. Plasma samples were collected at the following time-points in relation to dosing on Day 1: pre-dose (within 30 minutes before dosing) and at 0.25, 0.5, 0.75, 1, 1.5, 2, 2.5, 3, 4, 5, 6, 8, 10, 12, 24, 30, 36, 48, 54, 60, and 72 hours post-dose, and were analysed for 2-((3-(4-cyanonaphthalen-1-yl)pyridine-4-yl)thio)-2-methylpropanoic acid content. A summary of the mean plasma pharmacokinetic parameters following administration of the immediate release compositions at various doses of 2-((3-(4-cyanonaphthalen-1-yl)pyridine-4-yl)thio)-2-methylpropanoic acid are provided in Table 8.

TABLE 8
Geometric Mean (95% CI) Plasma Pharmacokinetics of 2-((3-(4-
cyanonaphthalen-1-yl)pyridine-4-yl)thio)-2-methylpropanoic acid following a Single Dose
at Various Dose Levels under Fed or Fasted Conditions

| Dose | | | | Tₘₐₓ* | Cₘₐₓ | AUC₀₋₂₄ | AUc_{∞} | t_{1/2} |
|---|---|---|---|---|---|---|---|---|
| (mg) | Food N | Group | | (hr) | (ug/mL) | (μg · hr/mL) | (μg · hr/mL) | (hr) |
| 2 | Fasted | 5 | Geomean | 0.500 | 0.0364 | 0.0388 | 0.0450 | 12.1 |
| | N = 6 | | (95% CI) | (0.250-0.500) | (0.0224-0.0592) | (0.0298-0.0506) | (0.0347-0.0583) | (7.61-19.2) |
| 5 | Fasted | 4 | Geomean | 0.625 | 0.0729 | 0.102 | 0.121 | 14.2 |
| | N = 6 | | (95% CI) | (0.500-0.750) | (0.0537-0.0989) | (0.0891-0.117) | (0.108-0.135) | (10.7-18.8) |
| | Fed | 6 | Geomean | 1.25 | 0.0457 | 0.0752 | 0.0928 | 12.7 |
| | N = 6 | | (95% CI) | (0.750-2.50) | (0.0345-0.0606) | (0.0595-0.0951) | (0.0718-0.120) | (9.73-16.7) |
| 20 | Fasted | 1 | Geomean | 0.500 | 0.384 | 0.463 | 0.540 | 10.9 |
| | N = 6 | | (95% CI) | (0.250-1.50) | (0.268-0.550) | (0.397-0.540) | (0.469-0.623) | (6.15-19.2) |
| | Fed | 3 | Geomean | 1.25 | 0.181 | 0.350 | 0.415 | 13.8 |
| | N = 6 | | (95% CI) | (1.00-2.50) | (0.0921-0.357) | (0.234-0.523) | (0.282-0.611) | (7.88-24.0) |
| 40 | Fasted | 2 | Geomean | 0.750 | 0.760 | 1.07 | 1.27 | 9.51 |
| | N = 6 | | (95% CI) | (0.250-1.00) | (0.493-1.17) | (0.701-1.64) | (0.873-1.84) | (7.81-11.6) |

*Tₘₐₓ is represented by median (range);
^{$}Body weight normalized parameter

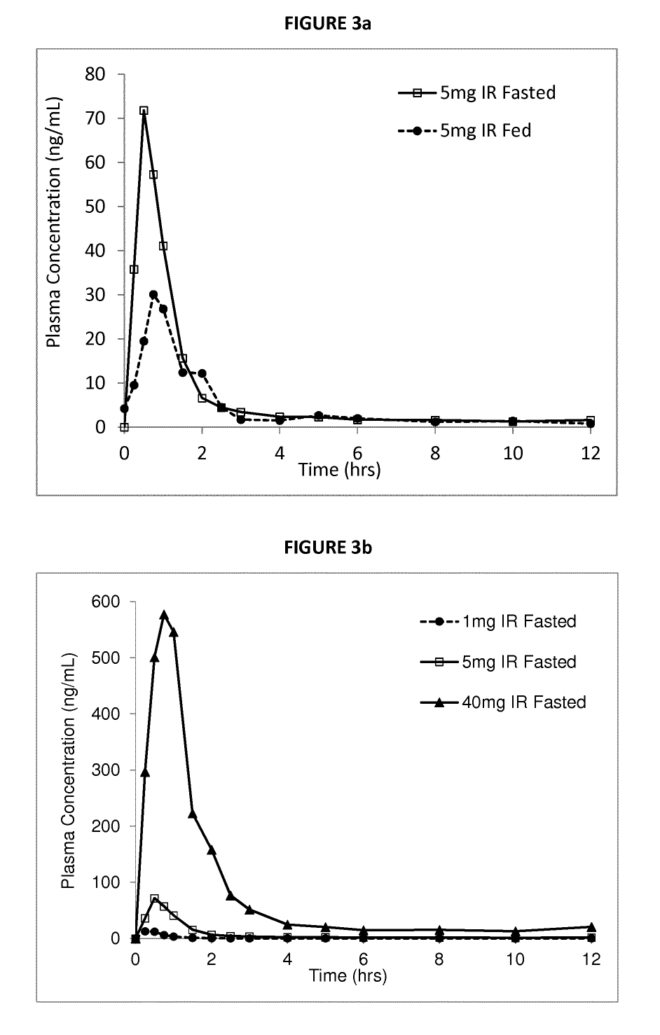

The mean plasma concentration-time profiles for the IR formulations under fed and fasted conditions are depicted in FIGS. 3a and 3b. Absorption of 2-((3-(4-cyanonaphthalen-1-yl)pyridine-4-yl)thio)-2-methylpropanoic acid following a single oral dose under fasted conditions was rapid. For example, at the 5 mg dose the geometric mean maximum plasma concentration (Cₘₐₓ) achieved is approximately 73 ng/ml and the time at which the peak plasma concentration is observed (Tₘₐₓ) is in the range of approximately 0.25-1.5 hours (median 0.6 hours). When 2-((3-(4-cyanonaphthalen-1-yl)pyridine-4-yl)thio)-2-methylpropanoic acid was administered with food, slightly slower absorption and lower exposure were observed. Plasma exposures of 2-((3-(4-cyanonaphthalen-1-yl)pyridine-4-yl)thio)-2-methylpropanoic acid displayed dose proportional increases in the 1 mg to 40 mg dose range. Important pharmacodynamic parameters related to the serum Uric Acid lowering effects are shown in the following two tables:

| | AUC₀₋₂₄ | Mean % change in sUA from |
|---|---|---|
| Dose/Condition | (ng · hr/mL) | predose¹ (%) |
| 2 mg/Fasted | 38.8 | 8% |
| 5 mg/Fasted | 102 | 15% |
| 20 mg/Fasted | 463 | 43% |
| 40 mg/Fasted | 1070 | 58% |

¹% sUA change mean maximum observed percentage change from pre-dose in serum urate concentrations (Eₘₐₓ)

| | | | | Urine Urate Excretion¹ | | | |
|---|---|---|---|---|---|---|---|
| | | AUC₀₋₂₄ | Cₘₐₓ/ | UUE | UUE | UUE | 0-6 hrs/ |
| Dose/Condition | Cₘₐₓ | (ng · hr/mL) | AUC₀₋₂₄ | 0-6 hrs | 6-12 hrs | 12-24 hrs | 0-24 hrs |
| 5 mg/Fasted | 72.9 | 102 | 0.72 | 509 | 231 | 347 | 0.468 |

¹Urine urate excretion (UUE) is measured as mg of urate per urine collection period.

### EXAMPLE 9: PHASE I/II CLINICAL TRIALS—MODIFIED RELEASE FORMULATIONS (MR1-5)

A Phase 1, randomized study to evaluated the PK, PD, and safety and tolerability of 2-((3-(4-cyanonaphthalen-1-yl)pyridine-4-yl)thio)-2-methylpropanoic acid MR formulations in n=12 healthy adult male subjects in fasted and fed states. This study evaluated a total of 5 MR formulations at a 5 mg dose. The MR formulations tested are those described in Examples 2-6. Plasma samples for PK analysis were collected at the following time points: Pre-dose (within 30 minutes before dosing) and at 0.25, 0.5, 0.75, 1, 1.5, 2, 2.5, 3, 4, 5, 6, 8, 10, 12, 24, 30, 36, 48, 54, 60 and 72 hours post-dose. A summary of the mean plasma pharmacokinetic parameters following administration of the MR compositions of 2-((3-(4-cyanonaphthalen-1-yl)pyridine-4-yl)thio)-2-methylpropanoic acid are provided in Table 9.

TABLE 9
Summary Plasma Pharmacokinetics of 2-((3-(4-cyanonaphthalen-1-yl)pyridine-4-
yl)thio)-2-methylpropanoic acid following a 5 mg Single Dose in various MR Formulations
under Fed or Fasted Conditions (Geometric Mean (95% CI))

| | | | Tₘₐₓ¹ | Cₘₐₓ | AUC₀₋₂₄ | AUC_{∞} | t_{1/2} |
|---|---|---|---|---|---|---|---|
| Form | Food | n | (hr) | (ng/mL) | (ng · hr/mL) | (ng · hr/mL) | (hr)² |
| MR1 | Fasted | 12 | 1.75 | 21.3 | 96.7 | 131 | 18.0 |
| | | | (1.00-3.00) | (16.6-27.3) | (77.3-121) | (96.1-178) | (11.0-29.3) |
| | Fed | 5 | 2.00 | 27.1 | 91.9 | 128 | 19.0 |
| | | | (1.00-2.50) | (16.2-45.6) | (71.6-118) | (79.6-206) | (10.5-34.2) |
| MR2 | Fasted | 12 | 1.50 | 32.2 | 116 | 145 | 13.4 |
| | | | (0.750-2.50) | (23.1-44.7) | (88.3-152) | (103-204) | (10.1-17.6) |
| | Fed | 6 | 2.00 | 47.2 | 123 | 154 | 15.3 |
| | | | (1.00-4.00) | (31.5-70.7) | (88.7-171) | (97.3-243) | (9.78-23.8) |
| MR3 | Fasted | 12 | 2.25 | 56.6 | 113 | 133 | 12.7 |
| | | | (0.750-6.00) | (42.4-75.6) | (91.8-139) | (107-165) | (10.2-15.8) |
| | Fed | 10 | 5.00 | 45.8 | 114 | 134 | 15.3 |
| | | | (2.50-6.00) | (32.9-63.7) | (87.2-149) | (104-173) | (11.7-19.9) |
| MR4 | Fasted | 12 | 2.25 | 7.40 | 46.2 | 68.5 | 15.4 |
| | | | (0.500-4.00) | (5.98-9.16) | (40.9-52.3) | (59.3-79.2) | (11.1-21.3) |
| | Fed | 5 | 2.00 | 8.65 | 44.3 | 57.4 | 10.2 |
| | | | (1.00-3.00) | (4.87-15.4) | (30.2-65.1) | (36.0-91.6) | (6.08-17.2) |
| MR5 | Fasted | 11 | 3.00 | 7.66 | 39.2 | 60.0 | 15.0² |
| | | | (1.00-4.00) | (5.67-10.4) | (29.2-52.7) | (44.6-80.8) | (11.3-20.0) |
| | Fed | 6 | 4.50 | 6.59 | 26.0 | 61.3 | 28.4 |
| | | | (2.00-5.00) | (4.62-9.38) | (21.7-31.1) | (34.6-109) | (11.5-70.3) |

¹Values are presented as median (range);
²Half lives in more than half subjects were calculated from a period of <2 calculated half-lives and deemed unreliable.

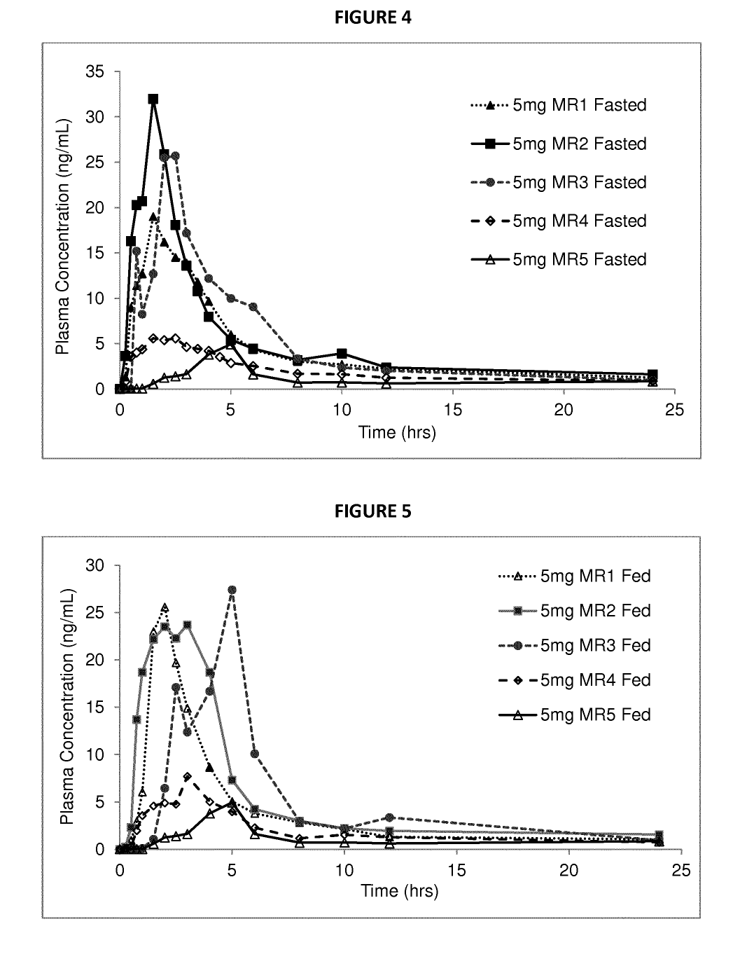

The mean plasma concentration-time profile for each formulation under fasted conditions is depicted in FIG. 4 and the profile for each formulation under fed conditions is depicted in FIG. 5.

As described above, a total of 5 modified-released formulations (MR1, MR2, MR3, MR4 and MR5) were evaluated in this study. Following a single oral 5 mg dose of 2-((3-(4-cyanonaphthalen-1-yl)pyridine-4-yl)thio)-2-methylpropanoic acid in these formulations under fasted conditions, 2-((3-(4-cyanonaphthalen-1-yl)pyridine-4-yl)thio)-2-methylpropanoic acid was readily absorbed from the MR1 and MR2 formulations (median Tmax 1.50-1.75 hours) and relatively slower from the MR4 and MR5 formulations (median Tmax 2.25-3.00 hours) (see Table 9 and FIG. 4). Both the MR3 and MR5 formulations showed a noticeable lag time in the absence or presence of food. Plasma concentrations of 2-((3-(4-cyanonaphthalen-1-yl)pyridine-4-yl)thio)-2-methylpropanoic acid declined with average terminal half-life values of approximately 10-28 hours (Table 9).

Using the MR1 formulation as a reference, under fasted conditions, the MR2 and MR3 formulations generally exhibited higher (51-166% higher) Cₘₐₓ values while the MR4 and MR5 formulations showed lower (approximately 64-65% lower) Cₘₐₓ values (Table 9). AUC exposures for the MR2 and MR3 formulations were both comparable to MR1, while the MR4 and MR5 formulations showed only half (approximately 48-54%) of the AUC exposure of MR1. The ranking order across the five formulations is MR3>MR2>MR1>MR4=MR5 for Cₘₐₓ and MR3=MR2=MR1>MR4=MR5 for AUC. Food had no impact on the rank order for the relative bioavailability (Table 9). Important pharmacodynamic parameters related to the serum Uric Acid lowering effects are shown in the following two tables:

| | | | Mean % change in |
|---|---|---|---|
| | | AUC₀₋₂₄ | sUA from predose¹ |
| Formulation | Dose/Condition | (ng · hr/mL) | (%) |
| MR1 | 5 mg/Fasted | 96.7 | 23.7% |
| MR2 | 5 mg/Fasted | 116 | 24.7% |
| MR3 | 5 mg/Fasted | 113 | 18.6% |
| MR4 | 5 mg/Fasted | 46.2 | 14.8% |
| MR5 | 5 mg/Fasted | 39.2 | 12.4% |

¹% sUA change mean maximum observed percentage change from pre-dose in serum urate concentrations (Eₘₐₓ)

| | | | | Urine Urate Excretion¹ | | | |
|---|---|---|---|---|---|---|---|
| Formulation/ | | AUC₀₋₂₄ | | UUE | UUE | UUE | 0-6 hrs/ |
| Condition | Cmax | (ng · hr/mL) | Cmax/AUC | 0-6 hrs | 6-12 hrs | 12-24 hrs | 0-24 hrs |
| MR1/Fasted | 21.3 | 96.7 | 0.22 | 449 | 258 | 219 | 0.485 |
| MR2/Fasted | 32.2 | 116 | 0.28 | 423 | 259 | 229 | 0.464 |
| MR3/Fasted | 56.6 | 113 | 0.50 | 388 | 272 | 245 | 0.429 |
| MR4/Fasted | 7.4 | 46.2 | 0.16 | 324 | 276 | 251 | 0.381 |
| MR5/Fasted | 7.7 | 39.2 | 0.20 | 290 | 275 | 267 | 0.349 |

¹Urine urate excretion (UUE) is measured as mg of urate per urine collection period.

Efficacy of 2-((3-(4-cyanonaphthalen-1-yl)pyridine-4-yl)thio)-2-methylpropanoic acid in lowering serum uric acid is linearly related to the AUC. AUC achieved was formulation dependent.

### EXAMPLE 10: PHASE I CLINICAL TRIALS—MODIFIED RELEASE FORMULATION MR4 BIOAVAILABILITY WHEN DELIVERED AS 4×2.5 MG TABLETS AND SINGLE 10 MG TABLET

A Phase 1, randomized, open label, 4 way crossover PK and PD study in healthy adult male subjects designed to assess the relative bioavailability of 2-((3-(4-cyanonaphthalen-1-yl)pyridine-4-yl)thio)-2-methylpropanoic acid 2.5 mg MR tablets administered as a 10 mg dose (4×2.5 mg tablets) and a single 2-((3-(4-cyanonaphthalen-1-yl)pyridine-4-yl)thio)-2-methylpropanoic acid 10 mg MR tablet. The 10 mg MR tablet was prepared as described in Example 5a. This study also assessed the effect of a low fat and high fat meal on the PK and PD of 2-((3-(4-cyanonaphthalen-1-yl)pyridine-4-yl)thio)-2-methylpropanoic acid 10 mg MR tablets. Sixteen subjects were randomized to 1 of 4 treatment sequences. The treatments administered on Days 1 or 5 according to the randomization schedule were as follows:
Treatment A: 10 mg dose of 2-((3-(4-cyanonaphthalen-1-yl)pyridine-4-yl)thio)-2-methylpropanoic acid, administered as 4×2.5 mg ER tablets, in the fasted state.
Treatment B: 10 mg dose of 2-((3-(4-cyanonaphthalen-1-yl)pyridine-4-yl)thio)-2-methylpropanoic acid, administered as a single 10 mg ER tablet, in the fasted state.

The treatments administered on Days 9 or 13 according to the randomization schedule were as follows:
Treatment C: 10 mg dose of 2-((3-(4-cyanonaphthalen-1-yl)pyridine-4-yl)thio)-2-methylpropanoic acid, administered as a single 10 mg ER tablet, in the fed state (low-fat, high-calorie meal).
Treatment D: 10 mg dose of 2-((3-(4-cyanonaphthalen-1-yl)pyridine-4-yl)thio)-2-methylpropanoic acid, administered as a single 10 mg ER tablet, in the fed state (high-fat, high-calorie meal).

During Treatments A and B subjects were fasted overnight for at least 10 hours prior to the start of PD collections. Subjects also fasted overnight for at least 10 hours prior to study medication dosing.

During Treatment C, subjects received the same standardized low-fat, high calorie breakfast (800 to 1000 calories and approximately 15% to 20% fat content consumed in 30 minutes or less), within the 30 minutes prior to dosing. During Treatment D, subjects received the same standardized high-fat, high calorie breakfast (800 to 1000 calories and approximately 50% fat content consumed in 30 minutes or less), within the 30 minutes prior to dosing. Subjects were instructed to consume 100% of the meal. Upon completion of the study breakfast, no food was allowed for 4 hours after the administration of 2-((3-(4-cyanonaphthalen-1-yl)pyridine-4-yl)thio)-2-methylpropanoic acid.

A summary of the mean plasma pharmacokinetic parameters following administration of the MR4 compositions are provided in Table 10.

TABLE 10
Summary Plasma Pharmacokinetics of 2-((3-(4-cyanonaphthalen-1-
yl)pyridine-4-yl)thio)-2-methylpropanoic acid in healthy adult male subjects following
various treatments (Geometric Mean [95% Confidence Interval])

| Treatment | | | | | | | |
|---|---|---|---|---|---|---|---|
| Group | | Tₘₐₓ^{a} | Cₘₐₓ | AUC₀₋₂₄ | AUCₗₐₛₜ | AUC_{∞} | t_{1/2} |
| (Treatment) | N | (hr) | (ng/mL) | (ng · hr/mL) | (ng · hr/mL) | (ng · hr/mL) | (hr) |
| A | 15 | 2.00 | 14.1 | 87.9 | 119 | 131 | 16.5 |
| (4 × 2.5 mg, | | (1.00-6.00) | (11.7-16.8) | (74.1-104) | (96.8-146) | (105-164) | (11.6-23.4) |
| Fasted) | | | | | | | |
| B | 15 | 2.00 | 14.9 | 84.6 | 114 | 130 | 15.5 |
| (1 × 10 mg, | | (1.00-4.00) | (11.9-18.8) | (66.6-107) | (85.7-153) | (95.9-176) | (10.6-22.5) |
| Fasted) | | | | | | | |
| C | 15 | 2.00 | 11.8 | 69.6 | 97.8 | 108 | 15.4 |
| (1 × 10 mg, | | (1.00-6.00) | (9.23-15.1) | (55.9-86.7) | (77.3-124) | (84.4-139) | (11.6-20.4) |
| Low-fat Fed^{b}) | | | | | | | |
| D | 15 | 4.00 | 27.2 | 128 | 160 | 173 | 16.6 |
| (1 × 10 mg, | | (1.50-8.00) | (20.2-36.6) | (103-159) | (130-199) | (137-219) | (11.5-23.9) |
| High-fat Fed^{c}) | | | | | | | |

Abbreviations:
AUC₀₋₂₄, area under the concentration-time curve from time zero up to 24 hours postdose;
AUCₗₐₛₜ, area under the concentration-time curve from time zero to the quantifiable last sampling timepoint;
AUC_{∞}, area under the concentration-time curve from time zero to infinity,
Cₘₐₓ, maximum observed concentration;
Tₘₐₓ, time of occurrence of maximum observed concentration;
t_{1/2}, apparent terminal half-life;
^{a}Tₘₐₓₜ values are represented by median (range).
^{b}15% to 20% fat, 800 to 1000 calories.
^{c}50% fat, 800 to 1000 calories.

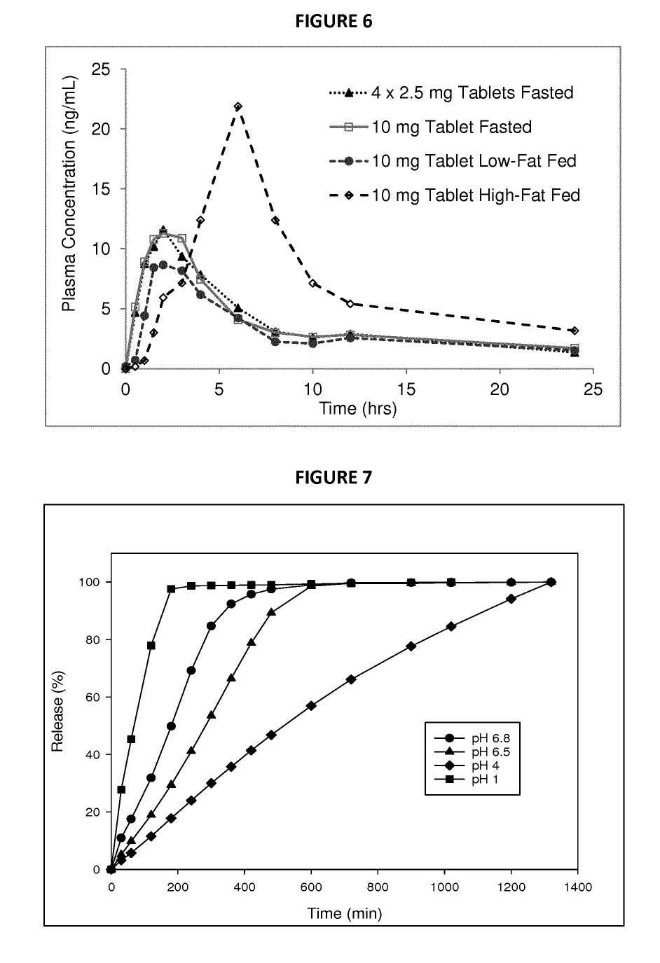

The mean plasma concentration-time profile for the formulation under fed and fasted conditions is depicted in FIG. 6.

The relative bioavailability of the 2-((3-(4-cyanonaphthalen-1-yl)pyridine-4-yl)thio)-2-methylpropanoic acid 10 mg MR tablet was 100% compared with the 2-((3-(4-cyanonaphthalen-1-yl)pyridine-4-yl)thio)-2-methylpropanoic acid 2.5 mg MR tablets administered as a total 10 mg dose in the fasted state, based on AUC∞. Geometric mean ratios and corresponding 90% CI for Cₘₐₓ and AUC∞ were within bioequivalence limits (80% to 125%). The sUA lowering following dosing with the 2-((3-(4-cyanonaphthalen-1-yl)pyridine-4-yl)thio)-2-methylpropanoic acid 10 mg MR tablet in the fasted state was comparable to dosing with 2-((3-(4-cyanonaphthalen-1-yl)pyridine-4-yl)thio)-2-methylpropanoic acid 2.5 mg MR tablets at 10 mg total dose.

Compared with the fasted state, a low-fat meal decreased the 2-((3-(4-cyanonaphthalen-1-yl)pyridine-4-yl)thio)-2-methylpropanoic acid Cₘₐₓ and AUC∞ exposures by approximately 21% and 17%, respectively. The sUA lowering following dosing with the low-fat meal was comparable to sUA lowering in the fasted state.

A high-fat meal increased the 2-((3-(4-cyanonaphthalen-1-yl)pyridine-4-yl)thio)-2-methylpropanoic acid Cₘₐₓ and AUC∞ exposures by 82% and 34%, respectively, for the 10 mg MR tablet compared with the fasted state. A high-fat meal enhanced the sUA lowering effect (an approximate 44% maximum reduction from predose value) compared with the fasted state (an approximate 32% maximum reduction from predose value). The enhanced sUA lowering under high-fat conditions is consistent with higher plasma drug exposures.

The sUA lowering achieved by administration of the formulations is shown in the following table:

| | | | AUC₀₋₂₄ | Mean % change |
|---|---|---|---|---|
| Formu- | | | (ng · | in sUA from |
| lation | Dose | Condition | hr/mL) | predose¹ |
| MR4 | 4 × 2.5 mg tablets | Fasted | 87.9 | 30.7% |
| MR4 | 1 × 10 mg tablet | Fasted | 84.6 | 31.5% |
| MR4 | 1 × 10 mg tablet | Low-Fat | 69.6 | 29.4% |
| | | Fed | | |
| MR4 | 1 × 10 mg tablet | High-Fat | 128 | 43.6% |
| | | Fed | | |

¹% sUA change mean maximum observed percentage change from predose in serum urate concentrations (Eₘₐₓ)

Efficacy of 2-((3-(4-cyanonaphthalen-1-yl)pyridine-4-yl)thio)-2-methylpropanoic acid in lowering serum uric acid is linearly related to the AUC.

### EXAMPLE 11: PROCESS FOR PREPARATION OF PELLET FORMULATIONS

Pellet formulations were prepared by a drug layering process. An inert core of a solid material of a mean size of from 100-700 μm was coated with 2-((3-(4-cyanonaphthalen-1-yl)pyridine-4-yl)thio)-2-methylpropanoic acid in a drug layering process. A solution or suspension containing said compound was sprayed onto the solid material and the solvent was evaporated. Examples of inert cores that can be used include microcrystalline cellulose such as Celphere CP-203 (200-300 μm), Celphere CP-305 (300-500 μm) or Celphere 507 (500-700 μm), silicon dioxide (sand) or sucrose.

After 2-((3-(4-cyanonaphthalen-1-yl)pyridine-4-yl)thio)-2-methylpropanoic acid has been layered onto the inert core, a film layer is formed to provide a release rate controlling membrane. The film layer contains a polymer such as ethylcellulose (EC) and/or hydroxypropylcellulose (HPC). The amount of HPC to EC is between 1-99%, such as 10-60% or 25-45% of the total polymer weight.

### Step 1: Coating of an Inert Core Pellet

A solution of the Agent is prepared in a concentration of from 1-30% w/w, such as from 5-15% w/w. The Agent is mixed with a binder, such as HPC, HPMC or other polymer and dispersed in a solvent. Examples of solvents that may be used are water or an alcohol such as ethanol, or a mixture thereof. The solution or suspension is held at a temperature of from 15° C. and 40° C. The solution or suspension of said compound is sprayed onto the core material in a fluidised bed equipment such as Aeromatic MP1, LabCC (Graniten LabCC) or Glatt GPCG at a temperature of from 50-100° C., such as from 35-80° C., or from 50-75° C., for example for s duration of 30-500 minutes. Batch sizes useful are typically from 10 g-400 kg. For a batch size of 1 kg, a spray rate of from 5-40 g/min is used.

It is also possible to use a crystallisation process without the need for a binder. In this case the crystalline compound can be dissolved in a solvent and then re-crystallised onto the cores/seeds in the fluid bed. This may be initiated or effected with or without seeding with crystals of said compound and can be performed in one step or be divided in several sub-bathes.

Step 2, Polymer Coating of Pellets from Step 1

The pellet granules formed in step 1 are coated with a polymer such as ethyl cellulose (EC), hydroxypropyl cellulose (HPC) or a mixture thereof. In one embodiment, the mixture contains HPC in a quantity of from 0 to 100%, such as 10 to 60%, or 20 to 50% of the total amount of the coating polymer. The polymer and/or the mixture thereof is dissolved in a solvent such as water, a ketone or an alcohol such as ethanol and/or mixtures thereof. The solution is sprayed onto the granules in fluidized bed equipment such as Aeromatic MP1, LabCC or Glatt GPCG at a temperature of from 60-120° C., such as from 75-100° C. The solution is sprayed onto the granules for a sufficient period of time, such as from 10 min to 400 minutes. The time required is dependent on the batch size and the desired thickness of the polymer film to achieve the desired Agent release profile. The batch size may be from 10 g up to 400 kg.

### Step 3, Capsule Filling or Tableting

The pellets comprising the compound 2-((3-(4-cyanonaphthalen-1-yl)pyridine-4-yl)thio)-2-methylpropanoic acid prepared according to step 2 may be filled into a capsule. Examples of a capsule material that may be used in accordance with the invention is hydroxypropyl methylcellulose or gelatine. Alternatively, the pellets can be formed into a tablet.

### EXAMPLE 12: PREPARATION OF PELLET FORMULATION (3-HOUR PROFILE)

A pellet formulation was prepared with the following composition:

Composition of modified release pellet capsules 5 mg

| | Quantity | |
|---|---|---|
| Components | (mg per capsule) | Supplier |
| Active compound¹ | 5.0 | |
| MCC spheres 0.15-0.3 mm | 22.2 | Asahi Kasei |
| HPMC 6 cps | 0.6 | Dow |
| HPC LF | 6.2 | Ashland |
| EC | 9.3 | Dow |
| Ethanol, 95 percent | qs | Kemetyl A |
| Water purified | qs | |
| HPMC capsule | NA | Qualicaps |

¹2-((3-(4-cyanonaphthalen-1-yl)pyridine-4-yl)thio)-2-methylpropanoic acid

This example formulation was prepared by a drug layering and polymer coating fluidized bed process and encapsulation. 2-((3-(4-cyanonaphthalen-1-yl)pyridine-4-yl)thio)-2-methylpropanoic acid was micronized using an air jet mill (Fluid Energy Mills). The resultant particle size D₁₀ was less than 1 μm, D₅₀ less than 5 μm and D₉₀ less than 20 μm.

A polymer solution of 15.0 g of HPMC 6 cps in 1350.0 g purified water was prepared. After a clear solution was obtained, 135.0 g micronized 2-((3-(4-cyanonaphthalen-1-yl)pyridine-4-yl)thio)-2-methylpropanoic acid was added. The resultant suspension was protected from light and stirred overnight. The suspension was held at RT ° C. Before spraying, the suspension was sieved through a 200 μm mesh. The spray rate was between 8-12 g suspension/min for the first 5 minutes and there after 10 to 20 g suspension/min for another 105 minutes. Inlet temperature was 72° C. 1250 g of the 2-((3-(4-cyanonaphthalen-1-yl)pyridine-4-yl)thio)-2-methylpropanoic acid/HPMC suspension was sprayed onto 500 g microcrystalline cellulose (MCC) powder spheres (Celphere CP-203 (150-300 μm)) in a fluidised bed equipment (LabCC3). The temperature of outlet air was approximately 30° C., fluidising air flow about 35 Nm³/h and an atomizer air pressure of approximately 2.5 bar. The product could be made in one or several steps depending on batch sizes.

A polymer solution of 57.6 g ethyl cellulose 10 cP (EC) and 38.4 g hydroxypropyl cellulose (HPC) dissolved in 1504 g of 95% ethanol was prepared. The drug layered pellets (150 g) were coated with the polymer solution in fluidized bed equipment at an outlet air temperature of approximately 42° C. with a spray rate of approximately 10-18 g/min. After spraying 1395 g of polymer solution the polymer coated pellets were dried for 10 minutes in fluidized bed equipment. See process parameters below.

| Process parameters | Ranges: |
|---|---|
| Inlet temperature | 72-74° C. |
| Outlet temperature | 42-60° C. |
| Fluidizing air flow | 35 Nm3/h |
| Spray Rate | 10-18 g/min |
| Atomization air pressure | 2.5 bar |
| Atomization air flow | 2.6-2.7 Nm³/h |

The polymer coated pellets were screened through a 710 μm sieve, assayed and then filled into hypromellose capsules, fill weight adjusted for dose to deliver 5 mg of 2-((3-(4-cyanonaphthalen-1-yl)pyridine-4-yl)thio)-2-methylpropanoic acid.

### EXAMPLE 13: PREPARATION OF PELLET FORMULATION (5-HOUR PROFILE)

A pellet formulation was prepared with the following composition: Composition of modified release pellet capsules 5 mg

Composition of modified release pellet capsules 5 mg

| | Quantity | |
|---|---|---|
| Components | (mg per capsule) | Supplier |
| Active compound¹ | 5.0 | |
| MCC spheres 0.15-0.3 mm | 22.2 | Asahi Kasei |
| HPMC 6 cps | 0.6 | Dow |
| HPC LF | 5.6 | Ashland |
| EC | 9.9 | Dow |
| Ethanol, 95 percent | qs | Kemetyl A |
| Water purified | qs | |
| HPMC capsule | NA | Qualicaps |

¹2-((3-(4-cyanonaphthalen-1-yl)pyridine-4-yl)thio)-2-methylpropanoic acid

This example formulation was prepared by a drug layering and polymer coating fluidized bed process and encapsulation. 2-((3-(4-cyanonaphthalen-1-yl)pyridine-4-yl)thio)-2-methylpropanoic acid was micronized using an air jet mill (Fluid Energy Mills). The resultant particle size D₁₀ was less than 1 μm, D₅₀ less than 5 μm and D₉₀ less than 20 μm.

A polymer solution of 15.0 g of HPMC 6 cps in 1350.0 g purified water was prepared. After a clear solution was obtained, 135.0 g micronized 2-((3-(4-cyanonaphthalen-1-yl)pyridine-4-yl)thio)-2-methylpropanoic acid was added. The spray rate was between 5-12 g suspension/min for the first 5 minutes and there after 10-20 g suspension/min for another 105 minutes. Inlet temperature was 72° C. 1250 g of the 2-((3-(4-cyanonaphthalen-1-yl)pyridine-4-yl)thio)-2-methylpropanoic acid/HPMC suspension was sprayed onto 500 g microcrystalline cellulose (MCC) powder spheres (Celphere CP-305 (300-500 μm) in fluidized bed equipment. The temperature of outlet air was approximately 30° C., fluidizing air flow approximately 35 Nm³/h and an atomizer air pressure approximately 2.5 bar. The product could be made in one or several steps depending on batch sizes.

A polymer solution of 61.4 g ethyl cellulose 10 cP (EC) and 34.6 g hydroxypropyl cellulose (HPC) dissolved in 1504 g of 95% ethanol was prepared. The drug layered pellets (150 g) were coated with the polymer solution in fluidized bed equipment at an outlet air temperature of approximately 42° C. with a spray rate of approximately 10-18 g/min. After spraying 1302.9 g of polymer solution the polymer coated pellets were dried for 10 minutes in fluidized bed equipment. See process parameters below.

| Process parameters: | Ranges |
|---|---|
| Inlet temperature | 72-74° C. |
| Outlet temperature | 42-60° C. |
| Fluidizing air flow | 35 Nm3/h |
| Spray Rate | 10-18 g/min |
| Atomization air pressure | 2.5 bar |
| Atomization air flow | 2.6-2.7 Nm³/h |

The polymer coated pellets were screened through a 710 μm sieve, assayed and then filled into hypromellose capsules, fill weight adjusted for dose to deliver 5 mg of 2-((3-(4-cyanonaphthalen-1-yl)pyridine-4-yl)thio)-2-methylpropanoic acid.

### EXAMPLE 14: PREPARATION OF PELLET FORMULATION (8-HOUR PROFILE)

A pellet formulation was prepared with the following composition:

### Composition of Modified Release Pellet Capsules 10 mg

Composition of modified release pellet capsules 10 mg

| | Quantity | |
|---|---|---|
| Components | (mg per capsule) | Supplier |
| Active Compound¹ | 10.0 | |
| MCC spheres 0.15-0.3 mm | 44.5 | Asahi Kasei |
| HPMC 6 cps | 1.1 | Dow |
| HPC LF | 10.1 | Ashland |
| EC | 20.9 | Dow |
| Ethanol, 95 percent | qs | Kemetyl A |
| Water purified | qs | |
| HPMC capsule | NA | Qualicaps |

¹2-((3-(4-cyanonaphthalen-1-yl)pyridine-4-yl)thio)-2-methylpropanoic acid

This example formulation was prepared by a drug layering and polymer coating fluidized bed process and encapsulation. 2-((3-(4-cyanonaphthalen-1-yl)pyridine-4-yl)thio)-2-methylpropanoic acid was micronized using an air jet mill (Fluid Energy Mills). The resultant particle size D₁₀ was less than 1 μm, D₅₀ less than 5 μm and D₉₀ less than 20 μm.

A polymer solution of 15.0 g of HPMC 6 cps in 1350.0 g purified water was prepared. After a clear solution was obtained, 135.0 g micronized 2-((3-(4-cyanonaphthalen-1-yl)pyridine-4-yl)thio)-2-methylpropanoic acid was added. The spray rate was between 5-12 g suspension/min for the first 5 minutes and there after 10-20 g suspension/min for another 105 minutes. Inlet temperature was 72° C. 1250 g of the 2-((3-(4-cyanonaphthalen-1-yl)pyridine-4-yl)thio)-2-methylpropanoic acid/HPMC suspension was sprayed onto 500 g microcrystalline cellulose (MCC) powder spheres (Celphere CP-305 (300-500 μm)) in fluidized bed equipment. The temperature of outlet air was approximately 30° C., fluidizing air flow approximately 35 Nm³/h and an atomizer air pressure approximately 2.5 bar. The product could be made in one or several steps depending on batch sizes.

A polymer solution of 64.8 g ethyl cellulose 10 cP (EC) and 31.3 g hydroxypropyl cellulose (HPC) dissolved in 1504 g of 95% ethanol was prepared. The drug layered pellets (150 g) were coated with the polymer solution in fluidized bed equipment at an outlet air temperature of approximately 42° C. with a spray rate of approximately 10-18 g/min. After spraying 1395 g of polymer solution the polymer coated pellets were dried for 10 minutes in a fluidized bed equipment. See process parameters below.

| Process parameters | Ranges |
|---|---|
| Inlet temperature | 72-74° C. |
| Outlet temperature | 42-60° C. |
| Fluidizing air flow | 35 Nm3/h |
| Spray Rate | 10-18 g/min |
| Atomization air pressure | 2.5 bar |
| Atomization air flow | 2.6-2.7 Nm³/h |

The polymer coated pellets were screened through a 710 μm sieve, assayed and then filled into hypromellose capsules, fill weight adjusted for dose to deliver 10 mg of 2-((3-(4-cyanonaphthalen-1-yl)pyridine-4-yl)thio)-2-methylpropanoic acid.

### EXAMPLE 15: PREPARATION OF PELLET FORMULATION (15-HOUR PROFILE)

A pellet formulation was prepared with the following composition:

Composition of modified release pellet capsules 10 mg

| | Quantity | |
|---|---|---|
| Components | (mg per capsule) | Supplier |
| Active compound¹ | 10 | |
| MCC spheres 0.15-0.3 mm | 44.5 | Asahi Kasei |
| HPMC 6 cps | 1.1 | Dow |
| HPC LF | 9.0 | Ashland |
| EC | 22.0 | Dow |
| Ethanol, 95 percent | qs | Kemetyl A |
| Water purified | qs | |
| HPMC capsule | NA | Qualicaps |

¹2-((3-(4-cyanonaphthalen-1-yl)pyridine-4-yl)thio)-2-methylpropanoic acid

This example formulation was prepared by a drug layering and polymer coating fluidized bed process and encapsulation. 2-((3-(4-cyanonaphthalen-1-yl)pyridine-4-yl)thio)-2-methylpropanoic acid was micronized using an air jet mill (Fluid Energy Mills). The resultant particle size D₁₀ was less than 1 μm, D₅₀ less than 5 μm and D₉₀ less than 20 μm.

A polymer solution of 15.0 g of HPMC 6 cps in 1350.0 g purified water was prepared. After a clear solution was obtained, 135.0 g micronized 2-((3-(4-cyanonaphthalen-1-yl)pyridine-4-yl)thio)-2-methylpropanoic acid was added. The spray rate was between 5-12 g suspension/min for the first 5 minutes and there after 10-20 g suspension/min for another 105 minutes. Inlet temperature was 72° C. 1250 g of the 2-((3-(4-cyanonaphthalen-1-yl)pyridine-4-yl)thio)-2-methylpropanoic acid/HPMC suspension was sprayed onto 500 g microcrystalline cellulose (MCC) powder spheres (Celphere CP-305 (300-500 μm)) in fluidized bed equipment. The temperature of outlet air was approximately 30° C., fluidizing air flow approximately 35 Nm³/h and an atomizer air pressure approximately 2.5 bar. The product could be made in one or several steps depending on batch sizes.

A polymer solution of 68.2 g ethyl cellulose 10 cP (EC) and 27.8 g hydroxypropyl cellulose (HPC) dissolved in 1504 g of 95% ethanol was prepared. The drug layered pellets (150 g) were coated with the polymer solution in fluidized bed equipment at an outlet air temperature of approximately 42° C. with a spray rate of approximately 10-18 g/min. After spraying 1395 g of polymer solution the polymer coated pellets were dried for 10 minutes in a fluidized bed equipment. See process parameters below.

| Process parameters: | |
|---|---|
| Inlet temperature | 72-74° C. |
| Outlet temperature | 42-60° C. |
| Fluidizing air flow | 35 Nm3/h |
| Spray Rate | 10-20 g/min |
| Atomization air pressure | 2.5 bar |
| Atomization air flow | 2.6-2.7 Nm³/h |

The polymer coated pellets were screened through a 425-710 μm sieve, assayed and then filled into hypromellose capsules, fill weight adjusted for dose to deliver 10 mg of 2-((3-(4-cyanonaphthalen-1-yl)pyridine-4-yl)thio)-2-methylpropanoic acid.

### EXAMPLE 16: PREPARATION OF PELLET FORMULATION USING MONO-ETHANOLAMINE SALT

A pellet formulation was prepared with the following composition:

Composition of MEA modified release pellet capsules 5 mg

| | Quantity | |
|---|---|---|
| Components | (mg per capsule) | Supplier |
| Active Compound¹ | 5 | |
| MCC spheres 0.3-0.5 mm | 25.77 | Asahi Kasei |
| HPMC 6 cps | 0.46 | Dow |
| PVP K30 | 4.04 | Sigma-Aldrich |
| EC | 12.79 | Dow |
| Ethanol, 95 percent | Qs | Kemetyl A |
| Magnesium stearate | 0.06 | Peter Greven |
| Water purified | Qs | Milli Q |
| HPMC capsule | NA | Qualicaps |

¹2-((3-(4-cyanonaphthalen-1-yl)pyridine-4-yl)thio)-2-methylpropanoic acid monoethanolamine

Cores from Celphere CP305 (Asahi Kasei, 0.3-0.5 mm) were used as the starting material. The API suspension used to coat the cores consisted of MilliQ water, micronized 2-((3-(4-cyanonaphthalen-1-yl)pyridine-4-yl)thio)-2-methylpropanoic acid mono-ethanolamine salt (MEA salt, prepared as described below) and HPMC 6 cps.

The MEA salt layered core pellets were manufactured to an MEA salt concentration of between 165 and 176 mg/g. The dry content of the suspension was 22%, 20% MEA salt and 2% HPMC 6 cps.

The suspension for the MEA salt layer coating was prepared by first dissolving HPMC in purified water using a magnetic stirrer overnight. Thereafter the MEA salt was added and the suspension was stirred prior to use. The suspension was kept stirring during the coating process.

The MEA salt layered core pellets were manufactured in bottom sprayed fluid bed equipment (MiniGlatt). Typical scale of manufacturing was 25 g cores and 118 g of coating suspension.

The ethanol based solution for the MR-films was prepared by adding EC/PVP to 95% Ethanol during stirring. The materials were left over night to dissolve. The coating was performed in a fluid bed equipment (MiniGlatt). Process parameters are seen below.

Process parameters for MEA salt layered seed/core pellets

| | |
|---|---|
| Tin | 70-75° C. |
| Tout | 40-60° C. |
| FF | 13 Nm3/h |
| Coat speed | 2-4.0 g/min |
| Atom. press | 1.0 bar |
| Atom. flow | 1.1-1.3 Nm³/h |

Process parameters MR coating of pellets

| | |
|---|---|
| Tin | 70-75° C. |
| Tout | 45-60° C. |
| FF | 11 Nm³/h |
| Coat speed | 4-6.0 g/min |
| Atom. press | 1.0 bar |
| Atom. flow | 0.4-0.5 Nm³/h |

Preparation of the Mono-Ethanolamine Salt

2-((3-(4-cyanonaphthalen-1-yl)pyridine-4-yl)thio)-2-methylpropanoic acid mono-ethanolamine salt was isolated from 2-((3-(4-cyanonaphthalen-1-yl)pyridine-4-yl)thio)-2-methylpropanoic acid n-butanolate according to the following procedure. To 2-((3-(4-cyanonaphthalen-1-yl)pyridine-4-yl)thio)-2-methylpropanoic acid n-butanolate ((34.80 g, 92.53 mmol) was added methyl ethyl ketone (167 mL) and dimethyl sulfoxide (42 mL). The resulting mixture was heated to 47-50° C. in order to form a solution. The solution was then clarified by filtration, and the resulting filtrate re-heated to 47-50° C. 2-Aminoethanol (6.1 mL, 100 mmol) was then added over at least 10 minutes, initiating the precipitation of the product from solution. The temperature was reduced to 0-10° C. over approximately 2 hours, and the product slurry stirred for 1 hour at this temperature range. The product was isolated by filtration, the filter cake washed twice with methyl ethyl ketone (2×70 mL) and dried in vacuo to constant weight at 60-65° C., yielding 2-((3-(4-cyanonaphthalen-1-yl)pyridine-4-yl)thio)-2-methylpropanoic acid mono-ethanolamine salt as a crystalline white solid (35.91 g, 86.37 mmol, 93.3%).

2-((3-(4-cyanonaphthalen-1-yl)pyridine-4-yl)thio)-2-methylpropanoic acid mono-ethanolamine salt can also be isolated from 2-((3-(4-cyanonaphthalen-1-yl)pyridine-4-yl)thio)-2-methylpropanoic acid according to the following procedure. To 2-((3-(4-cyanonaphthalen-1-yl)pyridine-4-yl)thio)-2-methylpropanoic acid (64.81 g, 181 mmol) was added methyl ethyl ketone (311 mL) and dimethyl sulfoxide (78 mL). The resulting mixture was heated to 47-50° C. in order to form a solution. The solution was then clarified by filtration, and the resulting filtrate re-heated to 47-50° C. 2-Aminoethanol (11.5 mL, 191 mmol) was then added over at least 10 minutes, initiating the precipitation of the product from solution. The temperature was reduced to 0-10° C. over approximately 2 hours, and the product slurry stirred for at least 30 minutes at this temperature range. The product was isolated by filtration, the filter cake washed twice with methyl ethyl ketone (2×65 mL) and dried in vacuo to constant weight at 60° C., yielding 2-((3-(4-cyanonaphthalen-1-yl)pyridine-4-yl)thio)-2-methylpropanoic acid mono-ethanolamine salt as a crystalline white solid (71.73 g, 172.3 mmol, 95.1%).

¹H NMR (400 MHz, DMSO-d6, 90° C.) 1.40 (d, J=9.6 Hz, 6H), 2.79 (t, J=5.5 Hz, 2H), 3.55 (t, J=5.5 Hz, 2H), 7.51 (d, J=8.5 Hz, 1H), 7.55 (d, J=7.4 Hz, 1H), 7.65 (td, J=1.1, 6.9, 7.6 Hz, 1H), 7.77-7.84 (m, 2H), 8.17 (d, J=7.4 Hz, 1H), 8.23 (d, J=8.4 Hz, 1H), 8.29 (s, 1H), 8.52 (d, J=5.4 Hz, 1H). Ratio of free acid: 2-aminoethanol by ¹H NMR 1:1.01.

### EXAMPLE 17: PREPARATION OF PELLET FORMULATION USING WATER-BASED COATING

A pellet formulation was prepared with the following composition:

Composition of modified release pellet capsules 5 mg

| | Quantity | |
|---|---|---|
| Components | (mg per capsule) | Supplier |
| Active Compound¹ | 5.0 | |
| MCC spheres 0.15-0.3 mm | 22.2 | Asahi Kasei |
| HPMC 6 cps | 0.54 | Dow |
| Eudragit NM30D | 2.75 | Evonik |
| Kollicoat IR | 0.775 | BASF |
| Talc | 1.175 | Sigma-Aldrich |
| Magnesium stearate | 0.06 | Peter Greven |
| Water purified | Qs | |
| HPMC capsule | NA | Qualicaps |

¹2-((3-(4-cyanonaphthalen-1-yl)pyridine-4-yl)thio)-2-methylpropanoic acid

2.47 g Kollicoat IR (polyvinyl alcohol/polyethylene glycol grafted copolymer, manufactured by BASF) and 3.75 g Talc powder was suspended in 64.52 g water. After stirring overnight, 29.25 g Eudragit NM30D dispersion was added. The dry content in the suspension was 15% w/w. The dispersion was held at RT ° C. Before spraying, the dispersion was sieved through a 200 μm mesh. The speed of the pump was between 1 and 2 g dispersion/min. Inlet temperature was 41° C. 35 g dispersion was sprayed onto 10 g of 2-((3-(4-cyanonaphthalen-1-yl)pyridine-4-yl)thio)-2-methylpropanoic acid drug layered core seeds (produced as described in Example 14) in a fluidised bed drier (MiniGlatt). The temperature of outlet air was about 25° C., fluidising air flow about 14 Nm3/h and an atomizer air pressure of about 1.6 bar giving 8.5% (w/w) active drug/MR granules.

| Process parameters | |
|---|---|
| Tin | 38-43° C. |
| Tout | 25-35° C. |
| FF | 14 Nm³/h |
| Coat speed | 1-2 g/min |
| Atom. press | 1.6 bar |
| Atom. flow | 1.6-1.8 Nm³/h |

### EXAMPLE 18: DISSOLUTION TESTING OF PELLET FORMULATIONS

### Methods

Dissolution of extended release pellets added as free pellets (not pellets in capsules) with a dose of 10 mg were performed in arrange of different pH media according to the general procedure of the United States Pharmacopeia Apparatus II (paddle). Aliquots of the dissolution test media were pumped in a closed loop for each individual vessel and filtered at specific time intervals and analyzed with a spectrophotometer equipped with 10 mm flow cell with UV detection at 303 nm with baseline correction by a three-point drop-line at 380-420 nm. The release of 2-((3-(4-cyanonaphthalen-1-yl)pyridine-4-yl)thio)-2-methylpropanoic acid was determined by comparing the UV responses of the sample chromatograms to the UV responses of a standard calibration curve. 500 mL dissolution media at 37° C. and a paddle speed of 100 rpm is used.

Dissolution media used with ionic strength (I) were applicable:

### pH 6.8 I=0.1: 50.0 mM KH2PO4+23.6 mM NaOH

### pH 6.8 I=0.025: 14.2 mM KH2PO4+5.4 mM NaOH

### pH 6.5: 10.4 mM Na2PO4, 3.3 mM NaOH, 106 mM NaCl,

### pH 6.0: 80.6 mM KH2PO4+9.7 mM NaOH

pH 5.5: 7.02 mM citric acid+19.91 mM Sodium citrate dihydrate
pH 4: 42.1 mM citric acid+27.3 mM Sodium citrate dihydrate

### pH 1: 0.1 M HCl

### Dissolution Results

FIG. 7 shows the dissolution profile for the 3-hour pellet formulation described in Example 12. Release rate is influenced by pH of the media. Note: the ionic strength of the pH 6.8 media was 0.1.

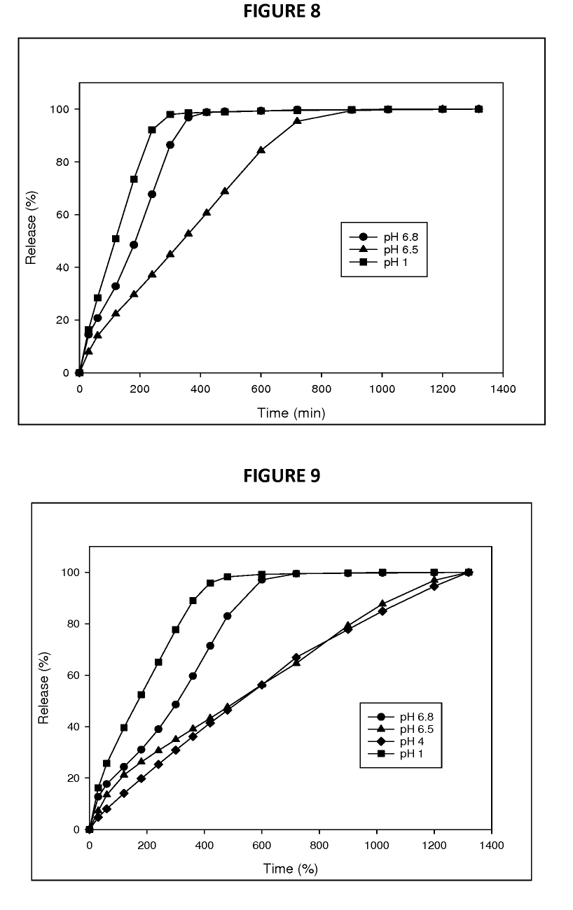

FIG. 8 shows the dissolution profile for the 5-hour pellet formulation described in Example 13. Release rate is influenced by pH of the media. Note: the ionic strength of the pH 6.8 media was 0.1.

FIG. 9 shows the dissolution profile for the 8-hour pellet formulation described in Example 14. Release rate is influenced by pH of the media. Note: the ionic strength of the pH 6.8 media was 0.1.

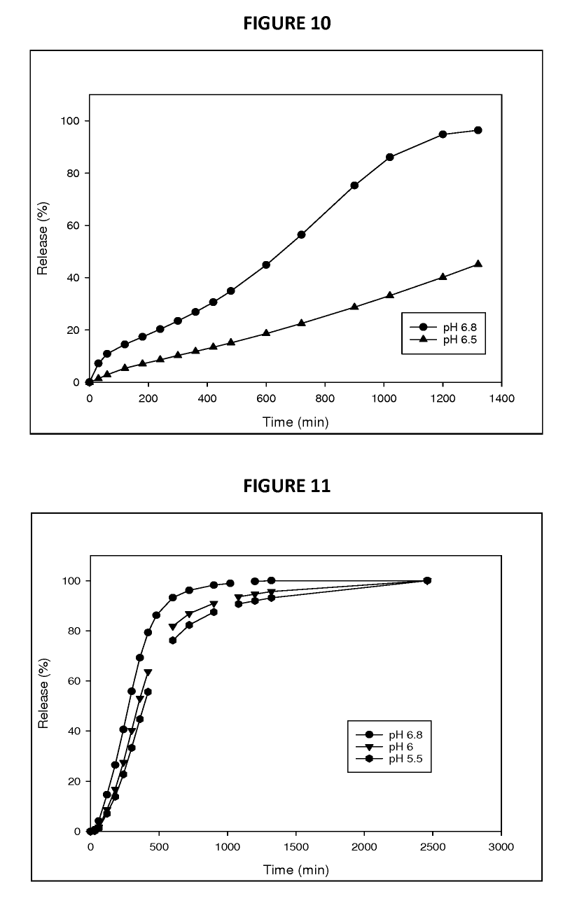

FIG. 10 shows the dissolution profile for the 15-hour pellet formulation described in Example 15. Release rate is influenced by pH of the media. Note: the ionic strength of the pH 6.8 media was 0.1.

FIG. 11 shows the dissolution profile for the mono-ethanolamine salt pellet formulation described in Example 16. Release is not significant influenced by the pH of the media. Note: the ionic strength of the pH 6.8 media was 0.1.

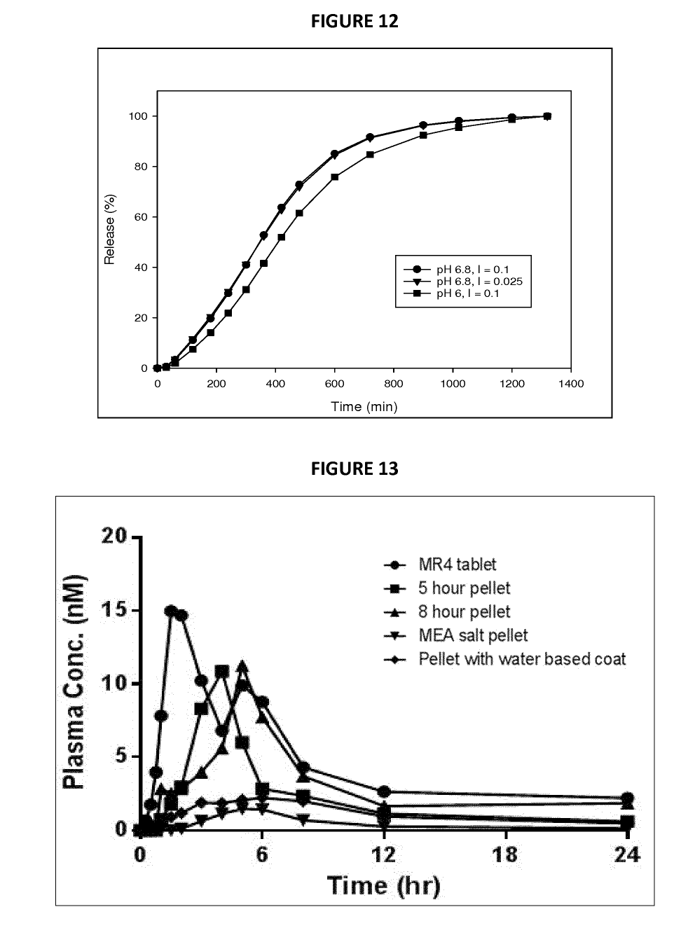

FIG. 12 shows the dissolution profile for a mono-ethanolamine salt pellet formulation prepared in accordance with Example 16 but with the one exception that the PVP and EC weight amounts were changed from 24% PVP K30 (76% EC) to 23% PVP K30 (77% EC). Release is not significantly influenced by the pH of the media or the ionic strengths tested.

### EXAMPLE 19: PK STUDY IN DOG MODELS—PELLET FORMULATIONS AND MR4

A pharmacokinetic study in Labrador dogs was performed under fasted conditions to compare the relative bioavailability of four different Pellet formulations with the MR4 tablet and an oral solution at the dose of 2.5 and 5 mg, which is equivalent to a human dose of 5 and 10 mg, respectively. The MR4 tablet tested in the study is described in Example 5. The pellet formulations tested in the study are described in Examples 12-17.

A lower relative bioavailability for all formulations was observed compared to the reference solution (see Table 11 and FIG. 13). The relative bioavailability of the 5 hour and 8 hour pellets were comparable to that of the MR4 tablet while the mono-ethanolamine salt and the water based coated pellet had significantly lower relative bioavailability compared to the other 5 hour and 8 hour pellets formulations.

TABLE 11
Mean plasma PK parameters of different formulations of the Agent in Labrador
dogs with acidic stomach pH under fasted conditions.

| | | | | | | Frel vs | Frel vs |
|---|---|---|---|---|---|---|---|
| | Dosage | Dose | Tmax | Cₘₐₓ | AUC0-24 | Formulation A | Solution |
| Formulation | form | (mg) | (hr) | (nM) | (nM * h) | (%) | (%) |
| Solution | solution | 5 | 0.24 | 95.9 | 219.6 | — | 100 |
| MR4 | Tablet | 5 | 1.9 | 17.4 | 110.4 | 100 | 55 |
| | (multiple) | | | | | | |
| 5 hour pellet | Capsule | 2.5 | 3.8 | 13.5 | 52.7 | 109 | 54 |
| 8 hour pellet | Capsule | 5 | 4.8 | 12.2 | 72.4 | 76 | 36 |
| MEA salt | Capsule | 5 | 5.0 | 1.7 | 10.5 | 9 | 5 |
| pellet | | | | | | | |
| Pellet with | Capsule | 5 | 5.5 | 3.3 | 27 | 25 | 14 |
| water-based | | | | | | | |
| coat | | | | | | | |

### EXAMPLE 20: PHASE I CLINICAL TRIAL—PELLET FORMULATIONS AND MR4

A Phase 1, randomized, open-label, 5-way crossover pharmacokinetic (PK) and pharmacodynamic (PD) study in healthy adult male subjects designed to assess the relative bioavailability of 2-((3-(4-cyanonaphthalen-1-yl)pyridine-4-yl)thio)-2-methylpropanoic acid 5 mg and 10 mg capsules and 2-((3-(4-cyanonaphthalen-1-yl)pyridine-4-yl)thio)-2-methylpropanoic acid 2.5 mg MR4 tablets administered as a 10 mg dose (2.5 mg×4). This study assessed the effect of a high-fat meal on the PK and PD of 2-((3-(4-cyanonaphthalen-1-yl)pyridine-4-yl)thio)-2-methylpropanoic acid. Plasma PK samples were collected at the following time points in relation to dosing of 2-((3-(4-cyanonaphthalen-1-yl)pyridine-4-yl)thio)-2-methylpropanoic acid: within 30 minutes prior to dosing and at 30 minutes, and 1, 1.5, 2, 3, 4, 6, 8, 10, 12, 24, 36, 48, and 72 hours post-dose. A summary of the mean plasma pharmacokinetic parameters following administration of the Pellet compositions of 2-((3-(4-cyanonaphthalen-1-yl)pyridine-4-yl)thio)-2-methylpropanoic acid are provided in Table 12.

TABLE 12
Summary Plasma Pharmacokinetics of 2-((3-(4-cyanonaphthalen-1-yl)pyridine-
4-yl)thio)-2-methylpropanoic acid following a Single Dose in various Pellet Formulations
under Fed or Fasted Conditions (Geometric Mean (95% CI))

| | | | | Food | | Geomean | |
|---|---|---|---|---|---|---|---|
| | | | | Effect Ratio | | PK Parameters | |
| | | Dose | Cₘₐₓ/AUC | (Fed/Fasted) | | Cₘₐₓ | AUC₀₋₂₄ |
| Formulation | Food | (mg) | Ratio | Cₘₐₓ | AUC_{∞} | (ng/mL) | (ng · hr/mL) |
| 5 h release | Fasted | 5 | 0.19 | 98.9% | 106% | 14.9 | 80.1 |
| | Fed^{a} | 5 | 0.17 | | | 15.0 | 86.7 |
| 8 h release | Fasted | 10 | 0.15 | 99.9% | 103% | 23.4 | 155 |
| | Fed^{a} | 10 | 0.14 | | | 23.3 | 163 |
| 15 h release | Fasted | 10 | 0.12 | 116% | 113% | 14.0 | 118 |
| | Fed^{a} | 10 | 0.12 | | | 16.3 | 142 |
| MR4, Cohort 1 | Fasted | 10 | 0.16 | — | — | 12.9 | 82.0 |
| MR4, Cohort 3 | Fasted | 10 | 0.15 | — | — | 13.2 | 89.6 |
| MR4 | Fasted | 10 | 0.18 | 182% | 134% | 14.9 | 84.6 |
| (10 mg tablet) | Fed^{a} | 10 | 0.21 | | | 27.2 | 128 |
| IR tablet | Fasted | 5 | 0.72 | 62.7% | 76.8% | 72.9 | 102 |
| | Fed^{b} | 5 | 0.61 | | | 45.7 | 75.2 |

^{a}high-fat meal;
^{b}low-fat meal

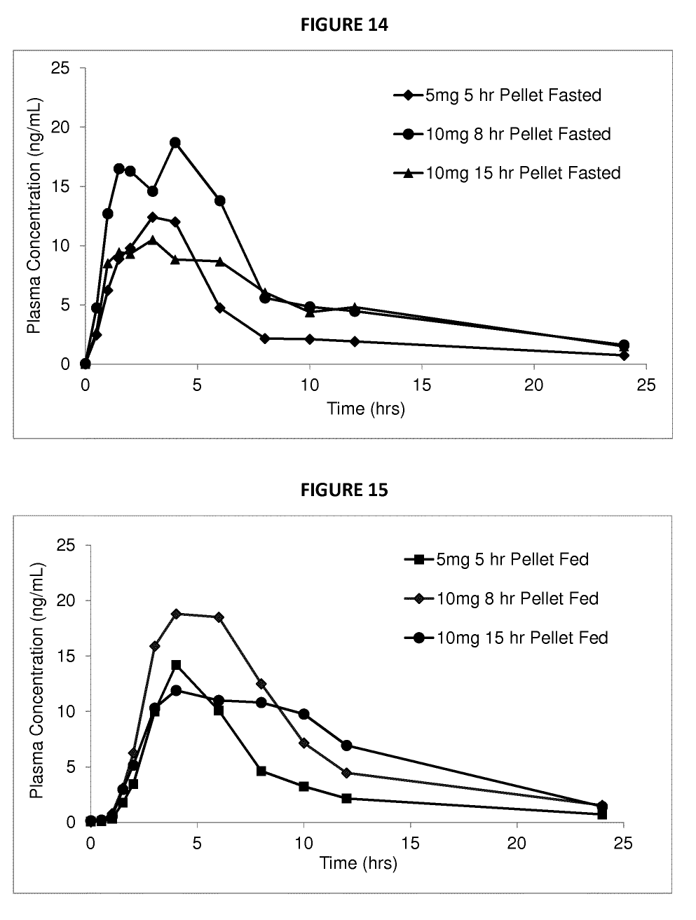

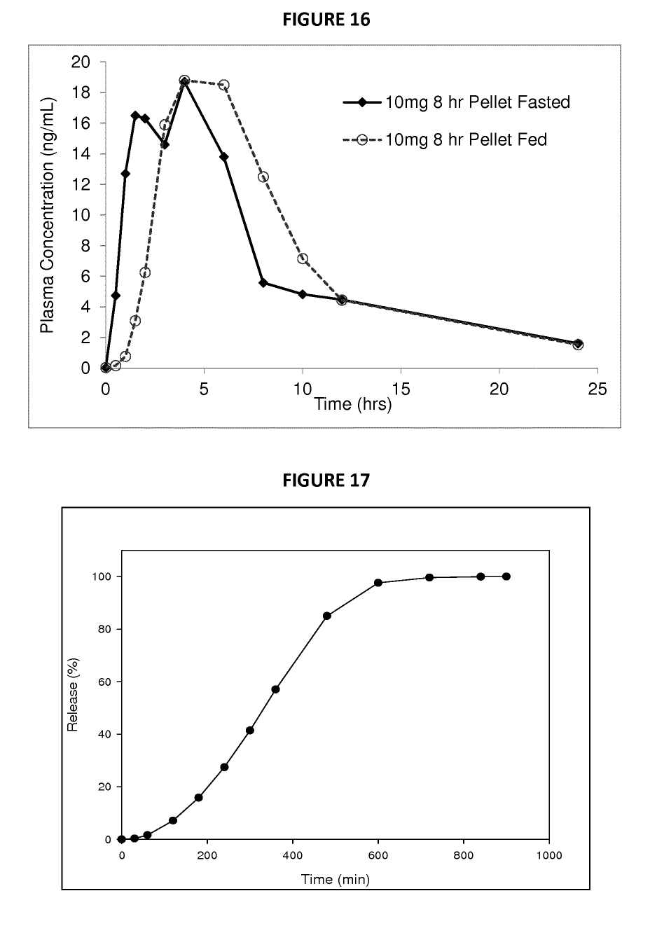

The mean plasma concentration-time profile for each formulation under fasted conditions is depicted in FIG. 14 and the profile for each formulation under fed conditions is depicted in FIG. 15. FIG. 16 shows the mean plasma concentration-time profile for the 8-hour pellet formulation described in Example 14 at a 10 mg dose in both the fasted and fed conditions.

Exposure of the 5 hr pellet formulation at a 5 mg dose was similar to that seen with a 10 mg dose of MR4. The 8 hr and 15 hr pellet formulations showed higher bioavailability than the same dose of the MR4 formulation, indicating an unexpectedly high extent of colonic absorption of the compound given its physicochemical properties. All pellet formulation showed no significant food effect and variability was similar to that for the MR4 formulation (measured as % CV). Rank order of Cₘₐₓ/AUC ratios is as follows: IR cap>>5 h>8 h≈MR4>15 h release form. The 5 hr pellet formulation had a higher Cₘₐₓ/AUC ratio to MR4 (0.19 versus 0.17 for MR4 in this study). Both the 8 hr and 15 hr pellet formulation had a lower Cₘₐₓ/AUC ratio than MR4 in this study (0.15 and 0.12 respectively).

The sUA lowering achieved by administration of the formulations are shown in the following tables:

| | | | | Mean % change |
|---|---|---|---|---|
| | | Condition | AUC₀₋₂₄ | in sUA from |
| Formulation | Dose | (Fasted/Fed) | (ng · hr/mL) | predose¹ (%) |
| MR4 (Cohort 1 | 10 mg | Fasted | 82.0 | 29.8% |
| and 3) | | | | |
| 5 hr Pellet | 5 mg | Fasted | 80.1 | 30.6% |
| 8 hr Pellet | 10 mg | Fasted | 155 | 42.5% |
| 15 hr Pellet | 10 mg | Fasted | 118 | 35.2% |

¹% sUA change mean maximum observed percentage change from pre-dose in serum urate concentrations (Eₘₐₓ)

### EXAMPLE 21: PREPARATION OF VARIOUS PELLET FORMULATIONS BY DRUG LAYERING PROCESS

A number of pellet formulations were prepared in accordance with the process described In Example 11. Table 13 provides details of composition and process parameters along with the dissolution time to 80% release in pH 6.8 media (ionic strength 0.1, 900 ml media, 100 rpm), tested in accordance with the dissolution method described in Example 18.

TABLE 13
Composition and process parameters for preparation of various pellet
formulations.

| | Coating | Amount | Batch | Coating | | | | | | Dose | Time 80% |
|---|---|---|---|---|---|---|---|---|---|---|---|
| | composition % | of film | size | speed | FF | AP | AF | Tᵢₙ | Tₒᵤₜ | (mg/g | released |
| Coating | w/w ratio | (wt %) | (g) | (g/min) | (Nm³/h) | (bar) | (Nm³/h) | (° C.) | (° C.) | pellets) | (min) |
| EC 10:HPC LF | 72:28 | 10.4 | 20 | 5.6 | 15 | 1.2 | 0.7 | 75 | 46 | 171 | 252 |
| EC 10:HPC LF | 68:32 | 22.7 | 100 | 19 | 35 | 2.5 | 2.6 | 75 | 43 | 158 | 552 |
| EC 10:HPC LF | 71:29 | 14 | 200 | 40.6 | 35 | 4.3 | 4.3 | 100 | 48 | 101 | 438 |
| EC 10:HPC LF | 71:29 | 12.5 | 600 | 40 | 35 | 4.3 | 4.2 | 100 | 44 | 102 | 540 |
| EC 10:HPC LF | 68:32 | 20.9 | 100 | 20.7 | 35 | 2.5 | 2.6 | 75 | 42 | 150 | 507 |
| EC 10:HPC LF | 68:32 | 18.9 | 200 | 37 | 35 | 4.7 | 4.1 | 100 | 45 | 162 | 390 |
| EC 10:HPC SSL | 70:30 | 24.5 | 6 | 3.8 | 12.5 | 1 | 0.4 | 73 | 41 | 118 | 150 |
| EC 10:HPC L | 75:25 | 17.4 | 6 | 4 | 13 | 1 | 0.4 | 75 | 44 | 120 | 1050 |
| EC 100:PVP | 70:30 | 20 | 10 | 4.5 | 12 | 1 | 0.4 | 69 | 43 | 100 | 30 |
| K30 | | | | | | | | | | | |
| EC10:PVP K30 | 76:24 | 33.2 | 10 | 5.5 | 11 | 1 | 0.4 | 80 | 46 | 116 | 354 |
| EC10:PVP K30 | 76:24 | 37.4 | 10 | 5.6 | 11 | 1 | 0.4 | 80 | 45 | 110 | 414 |

HPC LF supplied by Ashland.
HPC L and SSL supplied by Nisso.
Abbreviations:
Tᵢₙ (Inlet temperature),
Tₒᵤₜ (Outlet temperature),
FF (Fluidizing air flow),
AP (Pressure to atomise API or polymer solution) and
AF (Atomizer air flow).

### EXAMPLE 22: PREPARATION OF PELLET FORMULATION (8-HOUR PROFILE) AT DOSES 4.5, 6 AND 12 MG

Pellet formulations were prepared with the following compositions:

Composition of modified release pellet capsules 4.5, 6 and 12 mg

| | Quantity | Quantity | Quantity | |
|---|---|---|---|---|
| Components | (mg per capsule) | (mg per capsule) | (mg per capsule) | Supplier |
| Active Compound¹ | 4.5 | 6 | 12 | |
| MCC spheres 0.3-0.5 | 15.4 | 20.8 | 41.9 | Asahi Kasei |
| mm | | | | |
| HPMC 6 cps | 0.44 | 0.6 | 1.2 | Dow |
| HPC LF | 1.82 | 2.45 | 4.94 | Ashland |
| EC | 3.9 | 5.25 | 10.6 | Dow |
| Ethanol, 95 percent | Qs | Qs | Qs | Kemetyl A |
| Water purified | Qs | Qs | Qs | |
| Magnesium Stearate | 0.05 | 0.07 | 0.15 | |
| HPMC capsule | NA | NA | NA | Qualicaps |

¹2-((3-(4-cyanonaphthalen-1-yl)pyridine-4-yl)thio)-2-methylpropanoic acid

A polymer solution of 19.0 g of HPMC 6 cps in 1710.3 g water was prepared. After a clear solution was obtained, 171.0 g micronized 2-((3-(4-cyanonaphthalen-1-yl)pyridine-4-yl)thio)-2-methylpropanoic acid was added. The suspension was protected from light and stirred overnight. The suspension was held at RT ° C. Before spraying, the suspension was sieved through 200 μm mesh. The spray rate was between 8-11 g suspension/min for the first 5 minutes and there after 15-20 g suspension/min for another 111 minutes. Inlet temperature was 73° C. 1587.5 g the 2-((3-(4-cyanonaphthalen-1-yl)pyridine-4-yl)thio)-2-methylpropanoic acid/HPMC suspension was sprayed onto 500 g microcrystalline cellulose (MCC) powder spheres (Celphere CP-305 (300-500 μm)) in a fluidised bed equipment (LabCC3). The temperature of outlet air was approximately 30° C., fluidising air flow approximately 35 Nm³/h and an atomizer air pressure of approximately 2.5 bar. The product could be made in one or several steps depending on batch sizes.

100 g of these granules were coated with a solution of 20.0 g ethyl cellulose 10 cP (EC) and 9.4 g hydroxypropyl cellulose (HPC) dissolved in 460 g of 95% ethanol in a fluidised bed equipment (LabCC3) at a temperature of outlet air of 42° C. with a spray rate of in average 20 g solution/min. Process parameters were as follows:

| Process parameters | |
|---|---|
| Tin | 70-75° C. |
| Tout | 40-60° C. |
| FF | 35 Nm³/h |
| Coat speed | 18-22 g/min |
| Atom. press | 2.5 bar |
| Atom. flow | 2.6-2.7 Nm³/h |

Dissolution testing of the pellet formulation was carried out in accordance with the methods disclosed in Example 8 using pH 6.8 buffer (ionic strength 0.1, 50.0 mM KH2PO4+23.6 mM NaOH) at 37° C. using a paddles speed of 100 rpm. FIG. 17 shows the dissolution profile for the pellets produced as described above in this Example 22.

### EXAMPLE 23: PHASE I CLINICAL TRIAL—PELLET FORMULATIONS (8-HOUR PROFILE AT 4.5, 6 AND 12 MG DOSES)

A Phase 1, randomized, open-label, 3-way crossover pharmacokinetic (PK) study in healthy adult male subjects designed to assess the relative bioavailability of 2-((3-(4-cyanonaphthalen-1-yl)pyridine-4-yl)thio)-2-methylpropanoic acid 4.5, 6 and 12 mg capsules was conducted using the 8-hour profile formulations described in Example 22. A study to assess the effect of a high-fat meal on the PK of 2-((3-(4-cyanonaphthalen-1-yl)pyridine-4-yl)thio)-2-methylpropanoic acid was also conducted. Plasma PK samples were collected at the following time points in relation to dosing of 2-((3-(4-cyanonaphthalen-1-yl)pyridine-4-yl)thio)-2-methylpropanoic acid: within 30 minutes prior to dosing and at 30 minutes, and 1, 1.5, 2, 3, 4, 6, 8, 10, 12, 24, 36, 48, and 72 hours post-dose.

A summary of the mean plasma pharmacokinetic parameters following administration of the pellet compositions of 2-((3-(4-cyanonaphthalen-1-yl)pyridine-4-yl)thio)-2-methylpropanoic acid are provided in the following table:

| | | | Geomean PK Parameters | |
|---|---|---|---|---|
| Dose | | Cₘₐₓ/AUC | Cₘₐₓ | |
| (mg) | Food | Ratio | (ng/mL) | AUC₀₋₂₄ (ng · hr/mL) |
| 4.5 | Fasted | 0.173 | 11.8 | 68.0 |
| 6 | Fasted | 0.166 | 13.4 | 80.8 |
| 12 | Fasted | 0.170 | 28.6 | 168 |

A summary of the mean plasma pharmacokinetic parameters following administration of the pellet compositions of 2-((3-(4-cyanonaphthalen-1-yl)pyridine-4-yl)thio)-2-methylpropanoic acid in the food effect studies are provided in the following table:

| | | | Geomean | | Geomean | |
|---|---|---|---|---|---|---|
| | | | PK Parameters | | Fed Fasted | |
| | | Cₘₐₓ/AUC | Cₘₐₓ | AUC₀₋₂₄ | ratio (%) | |
| Dose (mg) | Food | Ratio | (ng/mL) | (ng · hr/mL) | AUCₗₐₛₜ | Cₘₐₓ |
| 6 | Fasted | 0.178 | 14.8 | 83.5 | 112 | 89.7 |
| | Fed | 0.136 | 13.3 | 98.2 | | |

No significant food effect for the pellet formulations with regards to Cₘₐₓ or AUC was observed. Furthermore, the Cₘₐₓ/AUC ratio decreased with food.

### EXAMPLE 24: PREPARATION OF A PELLET FORMULATION (8-HOUR PROFILE) AT DOSES 4.5, 6 AND 12 MG

Pellet formulations were prepared with the following compositions:

Compositions of modified release pellet capsules 4.5, 6 and 12 mg

| | Quantity | Quantity | Quantity | |
|---|---|---|---|---|
| Components | (mg per capsule) | (mg per capsule) | (mg per capsule) | Supplier |
| Active Compound¹ | 4.5 | 6 | 12 | |
| MCC spheres 0.5-0.7 | 29.0 | 38.7 | 77.4 | JRS |
| mm | | | | |
| HPMC 6 cps | 0.5 | 0.7 | 1.3 | Dow |
| HPC LF | 2.7 | 3.6 | 7.2 | Ashland |
| EC | 6.6 | 8.8 | 17.5 | Dow |
| Ethanol, 95 percent | Qs | Qs | Qs | Kemetyl A |
| Water purified | Qs | Qs | Qs | |
| Magnesium Stearate | 0.1 | 0.1 | 0.2 | |
| HPMC capsule | NA | NA | NA | Qualicaps |

¹2-((3-(4-cyanonaphthalen-1-yl)pyridine-4-yl)thio)-2-methylpropanoic acid

A polymer solution of 155 g of HPMC 6 cps in 13950 g water was prepared in excess. After a clear solution was obtained, 1395 g micronized 2-((3-(4-cyanonaphthalen-1-yl)pyridine-4-yl)thio)-2-methylpropanoic acid was added. The suspension was protected from light and stirred. The suspension was held at RT ° C. Before spraying, the suspension was sieved through 200 μm mesh. The spray rate was between 90.0-95.0 g suspension/min for the first 18 minutes and there after 96.0-97.0 g suspension/min for another 144 minutes. Inlet temperature was 74° C. 15530.0 g the 2-((3-(4-cyanonaphthalen-1-yl)pyridine-4-yl)thio)-2-methylpropanoic acid/HPMC suspension was sprayed onto 9000.0 g microcrystalline cellulose (MCC) powder spheres (Vivapur 500-700 μm (JRS Pharma)) in a fluidised bed equipment (FBC01). The temperature of outlet air was approximately 26.4° C. (24.4-39.2° C.), fluidising air flow approximately 183 Nm³/h and an atomizer air pressure of approximately 2.6 bar. The product could be made in one or several steps depending on batch sizes.

9000 g of these granules were coated with a solution of 1640 g ethyl cellulose 10 cP (EC) and 670 g hydroxypropyl cellulose (HPC) dissolved in 36190 g of 95% ethanol in a fluidised bed equipment (FBC01) at a temperature of outlet air of 23-45° C. with a spray rate of in average 241.0 g solution/min. Process parameters were as follows:

| Process parameters | |
|---|---|
| Tin | 100° C. |
| Tout | 23-45° C. |
| FF | 183 Nm³/h |
| Coat speed | 235.0-245.0 g/min (target 241.0 g/min) |
| Atom. press | 4.5-4.9 bar |
| Atom. flow (measured) | 21.5-23.0 Nm³/h |

Resulting modified release pellets were lubricated with magnesium stearate and filled into HPMC capsules.

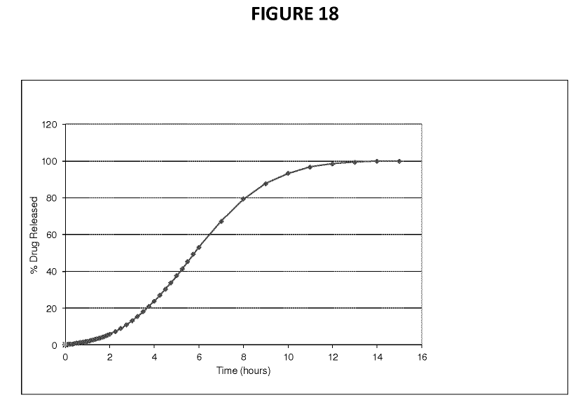

Dissolution testing of the pellet formulation was carried out in accordance with the methods disclosed in Example 8 using pH 6.8 buffer (ionic strength 0.1, 50.0 mM KH2PO4+23.6 mM NaOH) at 37° C. using a paddles speed of 100 rpm. FIG. 18 shows the dissolution profile for the pellets produced as described above in this Example 24.

1. A modified release pharmaceutical composition comprising the Agent, wherein said composition, after oral administration in the fasted state to a subject in need of treatment thereof exhibits at least one of the following:
a. produces in the subject a geometric mean maximum plasma concentration (Cₘₐₓ) of the Agent between 1 ng/ml and 50 ng/ml; and
b. produces a ratio of Cₘₐₓ/AUC₀₋₂₄ between 0.04 and 0.4.

2. A modified release pharmaceutical composition according to claim 1, wherein said composition, after oral administration in the fasted state to a subject in need of treatment thereof produces in the subject a ratio of Cₘₐₓ/AUC₀₋₂₄ between 0.04 and 0.4.

3. A modified release pharmaceutical composition according to claim 1, wherein said composition, after oral administration in the fasted state to a subject in need of treatment thereof exhibits both of the following:
a. produces in the subject a geometric mean maximum plasma concentration (Cₘₐₓ) of the Agent between 1 ng/ml and 40 ng/ml; and
b. produces a ratio of Cₘₐₓ/AUC₀₋₂₄ between 0.04 and 0.4.

4. A modified release pharmaceutical composition according to claim 2 or 3, wherein the ratio of Cₘₐₓ/AUC₀₋₂₄ is between 0.04 and 0.3.

5. A modified release pharmaceutical composition according to claim 2 or 3, wherein the ratio of Cₘₐₓ/AUC₀₋₂₄ is between 0.04 and 0.2.

6. A modified release pharmaceutical composition according to claim 2 or 3, wherein the ratio of Cₘₐₓ/AUC₀₋₂₄ is between 0.04 and 0.16.

7. A modified release pharmaceutical composition according to any one of claims 1 to 6, wherein the Agent is 2-((3-(4-cyanonaphthalen-1-yl)pyridine-4-yl)thio)-2-methylpropanoic acid.

8. A modified release pharmaceutical composition according to any one of claims 1 to 7, wherein the oral composition is administered to the subject to provide a dose of the Agent selected from a range of 0.5-20 mg, for example 0.5, 0.67, 0.75, 0.83, 1, 1.25, 1.5, 2, 2.5, 3, 3.3, 4.5, 5, 6, 7.5, 9, 10, 12, 15 and 20 mg.

9. A modified release pharmaceutical composition according to any one of claims 1 to 8, wherein the oral composition is administered to the subject to provide a dose of the Agent selected from 4.5, 6, 9 and 12 mg and the ratio of Cₘₐₓ/AUC₀₋₂₄ is between 0.04 and 0.16.

10. A modified release pharmaceutical composition according to any one of claims 1 to 9, wherein after oral administration at a dose of 10 mg in the fasted state to a subject in need of treatment thereof produces a AUC₀₋₂₄ of about 100 ng·hr/mL or more.

11. A modified release pharmaceutical composition according to any one of claims 1 to 10, wherein the pharmaceutical composition is a matrix dosage form or a multiparticulate system.

12. A modified release pharmaceutical composition according to claim 11, wherein the pharmaceutical composition is a matrix dosage form in the form of a tablet comprising a water erodible matrix.

13. A modified release pharmaceutical composition according to claim 11, wherein the pharmaceutical composition is a multiparticulate composition comprising a plurality of pellets or beads, where in each pellet or bead comprises a seed core layered with the Agent and coated with a polymeric material of the type useful for providing modified release of the Agent.

14. A method for treating a warm blooded animal, preferably a human, suffering from a condition treatable by the Agent comprising administering thereto a pharmaceutical composition according to any one of claims 1 to 13.

15. A method for treating disorders of uric acid metabolism selected from polycythemia, myeloid metaplasia, gout, a recurrent gout attack, gouty arthritis, hyperuricaemia, hypertension, a cardiovascular disease, coronary heart disease, heart failure, Lesch-Nyhan syndrome, Kelley-Seegmiller syndrome, acute or chronic kidney disease, kidney stones, kidney failure, joint inflammation, arthritis, urolithiasis, plumbism, hyperparathyroidism, psoriasis and sarcoidosisin, in a warm blooded animal, preferably a human, comprising administering thereto a pharmaceutical composition according to any one of claims 1 to 13.

16. The method according to claim 15, wherein the disorder of uric acid metabolism is gout.

17. A pharmaceutical composition according to any one of claims 1 to 13 for use as a medicament in the treatment of a condition treatable with the Agent.

18. A pharmaceutical composition according to any one of claims 1 to 13 for use as a medicament in the treatment of gout.

19. A pharmaceutical composition according to any one of claims 1 to 13, further comprising a xanthine oxidase inhibitor.

20. A pharmaceutical composition according to any one of claim 19, wherein the xanthine oxidase inhibitor is febuxostat.
